(19) United States
(12) Patent Application Publication
(10) Pub. No.: US 2006/0154984 A1
(43) Pub. Date: Jul. 13, 2006

(54) Therapeutic use of acyl glycerols and the nitrogen- and sulphur- containing analogues thereof

(76) Inventors: Raphael Darteil, Lille (FR); Karine Caumont-Bertrand, Frelinghien (FR); Jamila Najib, Santes (FR)

Correspondence Address: NIXON & VANDERHYE, PC, 901 NORTH GLEBE ROAD, 11TH FLOOR, ARLINGTON, VA, 22203, US

(21) Appl. No.: 10/542,512

(22) PCT Filed: Feb. 12, 2004
(86) PCT No.: PCT/FR04/00322

§ 371 Date: Jul. 18, 2005

(30) Foreign Application Priority Data
Feb. 12, 2003 (FR) 03/01691

(51) Int. Cl. A61K 31/21

(52) U.S. Cl. 514/513

(57) ABSTRACT

The invention relates to the use of acyl glycerols and the nitrogen- and sulfur-containing analogues thereof in the therapeutic field, particularly in human health. The inventive compounds have advantageous pharmacological properties and are particularly of use for the prevention or treatment of neurodegenerative diseases.

The invention relates to the use of acyl glycerols and the nitrogen- and sulfur-containing analogues thereof in therapy, particularly for the prevention and treatment of neurodegenerative diseases. The invention also relates to pharmaceutical compositions.

Neurodegenerative diseases are among the most common disorders of the central nervous system, together with vascular diseases and brain tumors. They currently affect a large and ever-growing population. The damage they cause is usually irreversible and progressively leads to degeneration of all or part of the nervous system.

An understanding of central nervous system functioning and dysfunctions makes it possible to develop novel therapeutic strategies for neurologic diseases. Despite more than ten years of effort in this field, the treatment of neurodegenerative diseases like multiple sclerosis, Alzheimer's disease or Parkinson's disease still remains a major challenge and a true public health concern.

The epidemiology of neurodegenerative diseases today is alarming. For instance, more than 300,000 people in France are afflicted with Alzheimer's disease, a number which will only grow as longevity increases. Diseases related to ageing are all the more prevalent with rising life expectancies, which today are 83 years for women and 74 years for men. Parkinson's disease afflicts some 100,000 people in France and 4 million people worldwide, while 60,000 cases of multiple sclerosis are diagnosed in France.

Current therapeutic strategies are derived from the fields of tissue regeneration or gene therapy, or are based on pharmacological methods as molecules are developed that are capable of regulating the expression of genes involved in disease development.

Alzheimer's disease (AD) is the most frequent neurodegenerative disorder. This pathology is characterized by extracellular deposits of βA4 amyloid protein leading to the formation of senile plaques and accumulation of hyperphosphorylated Tau protein which forms intracellular neurofibrillary tangles. Cholinergic neurons in the hippocampus are particularly affected but neuron loss also occurs in other regions of the brain. Loss of cells is accompanied by a loss of neurotransmitters, acetylcholine being the most important in AD. The resultant clinical signs include a progressive loss of brain function with dementia, memory loss and impaired cognitive and language skills.

Parkinson's disease (PD) is the second most frequent disorder after AD. It is characterized by a loss of dopaminergic neurons in the substantia nigra which, through neuronal projections, affects the neurons of the striatum. The symptoms resulting from the destruction of striatonigral pathways include rigidity, akinesia, dyskinesia and dementia.

Multiple sclerosis (MS) is a disorder which mainly afflicts young adults. It can be considered an autoimmune disease (the target being the oligodendrocytes) and is characterized by the formation of plaques of demyelination which cause the symptoms (paralysis, blindness, cognitive impairment, pain). The immune reaction observed in MS is characterized by phases of exacerbation and phases of remission the frequency and duration of which vary widely between patients.

While neurodegenerative diseases differ in terms of their etiology and pathophysiological mechanisms, the one feature they share in common is chronic inflammation, which develops and contributes to disease progression and neuron death through the release of neurotoxic molecules. Although neurons are capable of secreting inflammatory molecules, the glial cells (astrocytes and especially migroglia) play a particularly important role in this process. In pathological conditions, they acquire a so-called activated phenotype and release reactive oxygen species, nitric oxide (NO), proteases, and proinflammatory molecules (cytokines, prostaglandins, etc.).

Thus, microglial activation has been demonstrated in the amyloid plaques of AD, in the substantia nigra in brain of PD patients, and in the plaques of demyelination in MS. Proinflammatory molecules secreted by activated glia, or by neurons in pathological conditions are associated with the development and progression of neurodegenerative diseases. Cytokines like interleukin-1α (IL-1α), interleukin-6 (IL-6) and tumor necrosis factor α (TNF-α) are expressed in amyloid plaques and senile plaques, and in the brain of PD patients (Huell et al., 1995; Griffin et al., 1998; Boka et al., 1994; Mogi et al., 1994). High levels of proinflammatory molecules have also been detected during attacks of MS (Hohifeld, 1997) and cyclooxygenase 2 (COX-2) expression has been correlated with amyloid deposits, suggesting a role of prostaglandins in AD (Ho et al., 1999).

Oxidative stress also appears to play an important role in apoptosis of neurons observed in pathological conditions. For instance, elevated levels of lipoperoxidation and superoxide dismutase (SOD) activity have been observed in substantia nigra during the late stages of PD (Dexter et al., 1989; Saggu et al., 1989). The importance of oxidative stress and of inflammatory reactions is also illustrated by the observed increase in the NFκB transcription factor in dopaminergic neurons of PD patients (Hunot et al., 1997).

While research has long focused on cytokines and the responses of glial cells, neurons and lymphocytes in the case of MS, therapeutic tools such as protease inhibitors, inducible nitric oxide synthase (NOS), COX-2, and leukotriene antagonists have also been proposed, illustrating the extent of the inflammatory response in neurodegenerative pathologies.

Together, these observations have led to studies of the efficacy of anti-inflammatory drugs in in vitro models and in humans for the prevention or treatment of neurodegenerative pathologies.

Non-steroidal anti-inflammatory drugs (NSAIDS) like ibuprofen, aspirin and acetaminophen protect dopaminergic neurons and hippocampal neurons against toxicity induced by glutamate and β-amyloid protein (Casper et al., 2000; Bisaglia et al., 2002). Acetaminophen can also decrease cytokine and prostaglandin release by astrocytes previously stimulated with β-amyloid protein (Landolfi et al., 1998). Finally, ibuprofen treatment reduced both microglial activation and amyloid deposits in a transgenic mouse model (Lim et al., 2000).

Studies in humans have also demonstrated a neuroprotective role of NSAIDs. For instance, the risk of developing AD is considerably reduced in patients on chronic NSAID therapy (McGeer et al. 1996; Stewart et al., 1997). NSAIDs can also lessen the loss of cognitive skills and attenuate disease progression in Alzheimer patients (Rogers et al., 1993; Rich et al., 1995). The main target of NSAID action in brain, though not yet known, appears to be the microglia. In fact, the number of microglial cells associated with plaques in elderly patients decreased by 65% following NSAID treatment (McKenzie & Munoz, 1998). NSAIDs therefore have a positive effect in the treatment and prevention of neurodegenerative pathologies, but pose the major problem of causing serious side effects with long-term use.

The principal targets of NSAIDs are the cyclooxygenases (COX-1 and recently discovered COX-2). Said enzymes convert arachidonic acid to proinflammatory metabolites such as prostaglandins. Active therapeutic doses of NSAIDs are generally far above those required for their action on COX, which has led to the suggestion that other targets might be modulated by molecules like indomethacin or ibuprofen. Some authors recently showed that NSAIDs are capable of regulating gene expression through a direct interaction with members of the nuclear receptor family such as Peroxisome Proliferator-Activated Receptors or PPARs (Lehmann et al., 1997).

The PPARs are transcription factors which, after activation by their ligand, bind to specific sequences in the promoters of target genes and regulate the transcription of same. There are three PPAR isoforms (α, β/δ and γ). The discovery that leukotriene LTB4, a potent chemotactic agent, activates the PPARα receptor was the first evidence for a role of PPARs in inflammation (Devchland et al., 1996). Since then, it has been shown that PPAR α and γ can exert anti-inflammatory action by inhibiting the factors AP-1 and NFκB (Delerive et al., 2001). For instance, PPARα-deficient mice show a more severe response to inflammatory stimuli, further supporting the role of this receptor in controlling inflammatory mechanisms (Devchland et al., 1996). PPARα agonists are also capable of inhibiting cytokine expression in macrophage cultures (Combs et al., 2001) and the action of fibrates on IL6 expression is abolished in PPARα-deficient mice (Delerive et al, 1999). PPARα also appears to play a role by inhibiting COX-2 activity and thus decreasing the synthesis of inflammatory prostaglandins (Staels et al., 1998). Another interest of PPARs in terms of treating pathologies with an inflammatory component is their antioxidant potential. For instance, PPARα activation in elderly mice reduces tissue lipoperoxidation (Poynter & Daynes, 1998). Thus, the capacity to inhibit inflammatory responses by PPARs partly explains the therapeutic benefit of NSAIDs observed in the treatment of inflammatory pathologies.

NFκB and AP-1 are factors which control the majority of early genes involved in inflammatory disorders and NFκB is also involved in the oxidative response to stress. As PPARα antagonizes the action of these two factors, it is logical that agonists of said receptor can regulate the expression of a great many proteins involved in inflammatory reactions and oxidative stress in neurodegenerative pathologies.

PPAR expression has been studied mainly in peripheral tissues. The distribution of mRNA coding for said receptors has been studied in rat central nervous system, and PPARα expression was found in all cell types in rat brain. PPARγ mRNA is detected in the majority of cells but at lower levels (Cullingford et al., 1998). The presence of PPARα in oligodendrocytes suggests a role for said receptor in myelination, and an involvement in demyelinating pathologies such as multiple sclerosis (Kainu et al., 1994). PPAR expression has also been investigated in neuropathological conditions. PPARγ expression is high in pathological brain, pointing to a possible role in neurodegenerative pathologies (Kitamura et al., 1999).

PPAR agonists have anti-inflammatory and antioxidant potential, and PPARs are expressed in central nervous system cells. Moreover, the structure of PPAR agonists such as pioglitazone facilitates their passage across the blood-brain barrier, allowing them to act in brain (Maeshiba et al., 1997). As the inflammatory molecules expressed in brain are harmful to neurons, the effect of PPAR agonists was studied in models of neurodegeneration. PPARα agonists were found to produce dose-dependent inhibition of proinflammatory cytokine production by monocytes activated by β-amyloid protein (Combs et al., 2001). The same authors further showed that PPARγ agonists could also inhibit the production of inflammatory and neurotoxic molecules by β-amyloid-stimulated microglial cells, thereby positioning the PPAR agonists as potential therapeutic agents in the treatment of AD (Combs et al., 2000). PPARγ agonists are also capable of decreasing the expression of inducible NOS, reducing neuron death (Heneka et al., 2000) and inhibiting the development of EAE (experimental autoimmune encephalitis), an experimental model of multiple sclerosis (Diab et al., 2002; Natajaran & Bright, 2002). Finally, oral administration of a PPARγ agonist prevented the loss of dopaminergic neurons from substantia nigra in an experimental model of Parkinson's disease (Breidert et al., 2002).

PPARα therefore plays a role in inhibiting inflammatory molecules (by decreasing cytokine expression, by decreasing COX-2 expression) and in increasing antioxidant enzymes (catalase, superoxide dismutase), thereby reducing both oxidative stress and inflammatory reactions.

The inventive compounds have PPARα nuclear receptor activating properties and advantageous antioxidant and anti-inflammatory pharmacological properties.

The inventors have shown that the inventive compounds have advantageous properties enabling the prevention and treatment of Parkinson's disease.

The inventive compounds are represented by general formula (I): 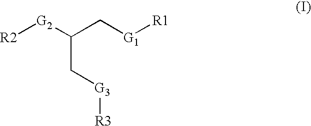
in which:
G1 represents an oxygen atom or a N—R group, G2 and G3 independently represent an oxygen atom, a sulfur atom or a N—R4 group, G1, G2 and G3 not simultaneously representing a N—R or N—R4 group,
R and R4 independently represent a hydrogen atom or a linear or branched alkyl group, saturated or not, optionally substituted, containing from 1 to 5 carbon atoms,
R1, R2 and R3, which are the same or different, represent a hydrogen atom, a CO—R5 group or a group corresponding to the formula CO—(CH₂)₂ₙ₊₁—X—R6, at least one of the groups R1, R2 or R3 being a group corresponding to the formula CO—(CH₂)₂ₙ₊₁—X—R6,
R5 is a linear or branched alkyl group, saturated or not, optionally substituted, possibly comprising a cyclic group, the main chain of which contains from 1 to 25 carbon atoms,
R6 is a linear or branched alkyl group, saturated or not, optionally substituted, possibly comprising a cyclic group, the main chain of which contains from 3 to 23 carbon atoms, preferably 10 to 23 carbon atoms and optionally one or more heterogroups, selected in the group consisting of an oxygen atom, a sulfur atom, a selenium atom, a SO group and a SO₂ group,
X is a sulfur atom, a selenium atom, a SO group or a SO₂ group,
n is a whole number comprised between 0 and 11.

In compounds represented by general formula (I) according to the invention, the R5 group or groups, which are the same or different, preferably represent a linear or branched alkyl group, saturated or unsaturated, substituted or not, the main chain of which contains from 1 to 20 carbon atoms, even more preferably 7 to 17 carbon atoms, still more preferably 14 to 17. In compounds represented by general formula (I) according to the invention, the R5 group or groups, which are the same or different, can also represent a lower alkyl group containing from 1 to 6 carbon atoms, such as in particular the methyl, ethyl, propyl, isopropyl, butyl, isobutyl, tertbutyl, pentyl or hexyl group.

In compounds represented by general formula (I) according to the invention, the R6 group or groups, which are the same or different, preferably represent a linear or branched alkyl group, saturated or unsaturated, substituted or not, the main chain of which contains from 3 to 23 carbon atoms, preferably 13 to 20 carbon atoms, even more preferably 14 to 17 carbon atoms, and still more preferably 14 carbon atoms.

Specific examples of saturated long chain alkyl groups for R5 or R6 are in particular the groups C₇H₁₅, C₁₀H₂₁, C₁₁H₂₃, C₁₃H₂₇, C₁₄H₂₉,C₁₅H₃₁, C₁₆H₃₃, C₁₇H₃₅. Specific examples of unsaturated long chain alkyl groups for R5 or R6 are in particular the groups C₁₄H₂₇, C₁₄H₂₅, C₁₅H₂₉, C₁₇H₂₉, C₁₇H₃₁, C₁₇H₃₃, C₁₉H₂₉, C₁₉H₃₁, C₂₁H₃₁, C₂₁H₃₅, C₂₁H₃₇, C₂₁H₃₉, C₂₃H₄₅ or the alkyl chains of eicosapentanoic (EPA) C_{20:5} (5, 8, 11, 14, 17) and docosahexanoic (DHA) C_{22:6} (4, 7, 10, 13, 16, 19) acids.

Examples of branched long chain alkyl groups are in particular the groups (CH₂)ₙ—CH(CH₃)C₂H₅, (CH═C(CH₃)—(CH₂)₂)_{nΔ}—CH═C(CH₃)₂ ou (CH₂)₂ₓ₊₁—C(CH₃)₂—(CH₂)_{n′″}—CH₃ (x being a whole number equal to or comprised between 1 and 11, n′ being a whole number equal to or comprised between 1 and 22, n″ being a whole number equal to or comprised between 1 and 5, n′″ being a whole number equal to or comprised between 0 and 22, and (2x+n′″) being less than or equal to 22, preferably less than or equal to 20).

As indicated earlier, the alkyl groups R5 or R6 can optionally comprise a cyclic group. Examples of cyclic groups are in particular cyclopropyl, cyclobutyl, cyclopentyl and cyclohexyl.

As indicated earlier, the alkyl groups R5 or R6 can optionally be substituted by one or more substituents, which are the same or different. The substituents are preferably selected in the group consisting of a halogen atom (iodine, chlorine, fluorine, bromine) and a —OH, ═O, —NO₂, —NH₂, —CN, —CH₂—OH, —O—CH₃, —CH₂OCH₃, —CF₃ and —COOZ group (Z being a hydrogen atom or an alkyl group, preferably containing from 1 to 5 carbon atoms).

The invention also relates to the optical and geometrical isomers of said compounds, the racemates, salts, hydrates thereof and the mixtures thereof.

In the case where G2 and G3 represent a N—R4 group, the R4 groups can be the same or different.

Preferred compounds in the spirit of the invention are compounds represented by general formula (I) in which (i) G1 represents a N—R group and (ii) the groups G2R2 and G3R3 do not simultaneously represent hydroxyl groups.

Compounds represented by formula (IA) are compounds corresponding to formula (I) according to the invention in which G1 and G3 represent oxygen atoms.

Compounds represented by formula (IB) are compounds corresponding to formula (I) according to the invention in which G1 represents a N—R group such as defined hereinabove.

Compounds corresponding to formula (IAa and IBa) are respectively compounds represented by formula (I) according to the invention in which a single one of the groups R1, R2 or R3 represents a hydrogen atom.

Compounds corresponding to formula (IAb and IBb) are respectively compounds represented by formula (I) according to the invention in which two of the groups R1, R2 or R3 represent a hydrogen atom.

The invention also encompasses the prodrugs of compounds represented by formula (I) which, after administration to a subject, are converted to compounds represented by formula (I) and/or metabolites of compounds represented by formula (I) according to the invention which display therapeutic activities similar to compounds represented by formula (I).

Moreover, in the group CO—(CH₂)₂ₙ₊₁—X—R6, X most preferably represents a sulfur or selenium atom and advantageously a sulfur atom.

Moreover, in the group CO—(CH₂)₂ₙ₊₁—X—R6, n is preferably comprised between 0 and 3, more specifically comprised between 0 and 2 and in particular is equal to 0.

In the compounds represented by general formula (I), R6 can contain one or more heterogroups, preferably 0, 1 or 2, more preferably 0 or 1, selected in the group consisting of an oxygen atom, a sulfur atom, a selenium atom, a SO group and a SO₂ group.

A specific example of a CO—(CH₂)₂ₙ₊₁—X—R6 group according to the invention is the group CO—CH₂—S—C₁₄H₂₉.

Preferred compounds in the spirit of the invention are therefore compounds represented by general formula (I) hereinabove in which at least one of the groups R1, R2 and R3 represents a CO—(CH₂)₂ₙ₊₁—X—R6 group in which X represents a sulfur or selenium atom and preferably a sulfur atom and/or R6 is a saturated and linear alkyl group preferably containing from 13 to 20 carbon atoms, preferably 14 to 17, more preferably 14 to 16, and even more preferably 14 carbon atoms.

Other particular inventive compounds are those in which at least two of the groups R1, R2 and R3 are CO—(CH₂)₂ₙ₊₁—X—R6 groups, which are the same or different, in which X represents a sulfur or selenium atom and preferably a sulfur atom.

Other particular compounds according to the invention are compounds represented by formula (IA) in which the group G2 advantageously represents an oxygen atom or a N—R4 group, preferably an oxygen atom. In said compounds, R2 advantageously represents a CO—(CH₂)₂ₙ₊₁—X—R6 group such as defined hereinabove. Moreover, when G2 is a N—R4 group, R4 preferably represents a hydrogen atom or a methyl group.

Other particular compounds according to the invention are compounds represented by formula (IB) in which G2 represents an oxygen or sulfur atom, and preferably an oxygen atom. In said compounds, R2 advantageously represents a group corresponding to the formula CO—(CH₂)₂ₙ₊₁—X—R6 such as defined hereinabove.

Particularly preferred compounds are compounds represented by general formula (IA) hereinabove in which:
G2 is an oxygen atom, and/or
R2 represents a CO—(CH₂)₂ₙ₊₁—X—R6 group such as defined hereinabove.

Particularly preferred compounds are compounds represented by general formula (IB) hereinabove in which:
G3 is a N—R4 group in which R4 is a hydrogen atom or a methyl group, and G2 is an oxygen atom, and/or
R2 represents a CO—(CH₂)₂ₙ₊₁—X—R6 group such as defined hereinabove.

Other preferred compounds are compounds represented by general formula (I) hereinabove in which R1, R2 and R3, which are the same or different, preferably the same, represent (i) a CO—(CH₂)₂ₙ₊₁—X—R6 group such as defined hereinabove, in which X represents a sulfur or selenium atom and preferably a sulfur atom and/or R6 is a saturated and linear alkyl group containing from 13 to 17 carbon atoms, preferably 14 to 16 carbon atoms, even more preferably 14 carbon atoms, in which n is preferably comprised between 0 and 3, and in particular is equal to 0. More specifically, preferred compounds are compounds represented by general formula (I) in which R1, R2 and R3 represent CO—CH₂—S—C₁₄H₂₉ groups.

Examples of preferred inventive compounds are given in FIGS. 1A and 1B.

Thus, the invention more particularly has as object the use of compounds represented by formula (I) selected in the group consisting of:

1-tetradecylthioacetylglycerol;

2-tetradecylthioacetylglycerol;

1,2,3-tritetradecylthioacetylglycerol;

1,2,3-tri-(4-dodecylthio)butanoylglycerol;

1,2,3-tri-(6-decylthio)hexanoylglycerol;

1,2,3-tritetradecylsulfoxyacetylglycerol;

1,2,3-tri-(tetradecylsulfonyl)acetylglycerol;

1,2,3-tri-tetradecylselenoacetylglycerol;

1,3-dipalmitoyl-2-tetradecylthioacetylglycerol;

1,3-dilinoleoyl-2-tetradecylthioacetylglycerol;

1,3-distearoyl-2-tetradecylthioacetylglycerol;

1,3-oleoyl-2-tetradecylthioacetylglycerol;

1,3-ditetradecanoyl-2-tetradecylthioacetylglycerol;

1-palmitoyl-2,3-ditetradecylthioacetylglycerol;

1-oleoyl-3-palmitoyl-2-tetradecylthioacetylglycerol;

1,3-dipalmitoyl-2-docosylthioacetylglycerol;

2-tetradecylthioacetamidopropane-1,3-diol;

2-tetradecylthioacetamido-1,3-ditetradecylthioacetyloxypropane;

1,3-ditetradecylthioacetyl-2-palmitoylglycerol;

1,3-diacetyl-2-tetradecylthioacetylglycerol;

1,3-dioctanoyl-2-tetradecylthioacetylglycerol;

1,3-diundecanoyl-2-tetradecylthioacetylglycerol;

1,3-ditetradecylthioacetoxy-2-(tetradecylthiomethyl)carbonylthiopropane;

3-(tetradecylthioacetylamino)propane-1,2-diol;

1-tetradecylthioacetylamino-2,3-(dipalmitoyloxy)propane;

3-tetradecylthioacetylamino-1,2-(ditetradecylthioacetyloxy)propane;

3-palmitoylamino-1,2-(ditetradecylthioacetyloxy)propane;

1,3-di(tetradecylthioacetylamino)propan-2-ol;

1,3-diamino-2-(tetradecylthioacetyloxy)propane;

1,3-ditetradecylthioacetylamino-2-(tetradecylthioacetyloxy)propane;

1,3-dioleoylamino-2-(tetradecylthioacetyloxy)propane;

1,3-ditetradecylthioacetylamino-2-(tetradecylthioacetylthio)propane; and

1-tetradecylthioacetylamino-2,3-di(tetradecylthioacetylthio)propane.

The invention also relates to the use of a compound represented by formula (I) for preparing a pharmaceutical composition intended to treat a neurodegenerative disease, such as in particular Parkinson's disease or Alzheimer's disease.

The invention also has as object a pharmaceutical composition comprising, in a pharmaceutically acceptable support, a compound represented by general formula (I) such as described hereinabove, optionally in association with another active therapeutic agent.

More specifically, the invention relates to a pharmaceutical composition comprising, in a pharmaceutically acceptable support, at least one compound represented by formula (I) such as described hereinabove intended for the treatment or prophylaxis of neurodegenerative pathologies and more particularly Parkinson's disease, Alzheimer's disease, or multiple sclerosis. In fact, it was found in a surprising manner that compounds represented by formula (I), concurrently display PPAR activator, antioxidant and anti-inflammatory properties and exhibit prophylactic and curative neuroprotective activity.

The invention further relates to the use of a compound such as defined hereinabove for preparing a pharmaceutical composition for carrying out a method of treatment or prophylaxis of neurodegenerative pathologies in humans or in animals, and more particularly Parkinson's disease, Alzheimer's disease or multiple sclerosis.

The invention also relates to a method of treatment of neurodegenerative diseases and more particularly Parkinson's disease, Alzheimer's disease or multiple sclerosis, comprising administering to a subject, particularly animal or in particular human, an effective dose of a compound represented by formula (I) or of a pharmaceutical composition such as defined hereinabove.

Advantageously, the compounds represented by formula (I) which are used are such as defined hereinabove.

The pharmaceutical compositions according to the invention advantageously comprise one or more pharmaceutically acceptable excipients or vehicles. Examples include pharmaceutically compatible saline, physiologic, isotonic, buffered solutions and the like, known to those skilled in the art. The compositions may contain one or more agents or vehicles selected from among dispersives, solubilizers, stabilizers, surfactants, preservatives, and the like. Agents or vehicles that may be used in the formulations (liquid and/or injectable and/or solid) comprise in particular methylcellulose, hydroxymethylcellulose, carboxymethylcellulose, Castor oil polyoxyl hydrogenated (product of the reaction of 45 moles of ethylene glycol with 1 mole of hydrogenated castor oil, which is sold and marketed by BASF under the name “Cremophor® RH40”), polyoxyl 35 Castor oil (product of the reaction of 35 moles of ethylene glycol with 1 mole of castor oil, which is sold and marketed by BASF under the name “Cremophor® EL”), polyethylene glycol 660 12-hydroxystearate (sold and marketed by BASF under the name “Solutol® HS15”), polysorbate 60 (sold and marketed by Croda under the name “Crillet® 3”), polysorbate 80 (sold and marketed by Croda under the name “Crillet® 4”), mannitol, gelatin, lactose, vegetable oils, acacia, and the like. The compositions may be formulated as injectable suspensions, gels, oils, tablets, suppositories, powders, gelatin capsules, capsules, and the like, possibly by means of pharmaceutical forms or devices allowing sustained and/or delayed release. For this type of formulation, an agent such as cellulose, carbonates or starches is advantageously used.

In this regard, the invention also relates to a pharmaceutical composition comprising, in a pharmaceutically acceptable support, at least one compound represented by formula (I) such as defined hereinabove in association with at least one compound selected in the group consisting of: Castor oil polyoxyl hydrogenated, polyoxyl 35 Castor oil, polyethylene glycol 660 12-hydroxystearate and polysorbate 60.

The compounds or compositions of the invention may be administered in different ways and in different forms. For instance, they may be administered systemically, by the oral route, parentally, by inhalation or by injection, such as for example by the intravenous, intramuscular, subcutaneous, transdermal, intra-arterial route, etc. For injections, the compounds are generally prepared in the form of liquid suspensions, which may be injected through syringes or by infusion, for example. In this respect, the compounds are generally dissolved in pharmaceutically compatible saline, physiologic, isotonic, buffered solutions and the like, known to those skilled in the art. For instance, the compositions may contain one or more agents or vehicles selected from among dispersives, solubilizers, emulsifiers, stabilizers, surfactants, preservatives, buffers, and the like. Agents or vehicles that may be used in the liquid and/or injectable formulations comprise in particular methylcellulose, hydroxymethylcellulose, carboxymethylcellulose, Cremophor® RH40, Cremophor® EL, Solutol® HS15, Crillet® 3, Crillet® 4, polysorbate 60, polysorbate 80, mannitol, gelatin, lactose, vegetable oils, acacia, liposomes, and the like.

The compositions may thus be administered in the form of gels, oils, tablets, suppositories, powders, gelatin capsules, capsules, aerosols, and the like, possibly by means of pharmaceutical forms or devices allowing sustained and/or delayed release. For this type of formulation, an agent such as cellulose, carbonates or starches is advantageously used.

The compounds may be administered orally in which case the agents-or vehicles used are preferably selected in the group consisting of water, gelatin, gums, lactose, starch, magnesium stearate, talc, an oil, polyalkylene glycol, and the like.

For parenteral administration, the compounds are preferably administered in the form of solutions, suspensions or emulsions in particular with water, oil or polyalkylene glycols to which, in addition to preservatives, stabilizers, emulsifiers, etc., it is also possible to add salts to adjust osmotic pressure, buffers, and the like.

It is understood that the injection rate and/or injected dose may be adapted by those skilled in the art according to the patient, the pathology, the mode of administration, etc. Typically, the compounds are administered at doses ranging from 1 μg to 2 g per dose, preferably from 0.1 mg to 1 g per dose. The doses may be administered once a day or several times a day, as the case may be. Moreover, the compositions of the invention may also comprise other active substances or agents.

The compounds of the invention can be prepared from commercially available products, by employing a combination of chemical reactions known to those skilled in the art.

According to a first method of the invention, compounds represented by formula (IA) in which G2 is an oxygen or sulfur atom, R1, R2 and R3, which are the same or different, represent a CO—R5 group or a CO—(CH₂)₂ₙ₊₁—X—R6 group, are obtained from a compound represented by formula (IA) in which G2 is respectively an oxygen or sulfur atom, R2 is a hydrogen atom and R1 and R3, which are the same or different, represent a CO—R5 or CO—(CH₂)₂ₙ₊₁—X—R6 group, and a compound corresponding to the formula A°-CO-A in which A is a reactive group selected for example in the group consisting of OH, Cl, O—CO-A° and OR″, R″ being an alkyl group, and A° is the R5 group or the (CH₂)₂ₙ₊₁—X—R6 group, possibly in the presence of coupling agents or activators known to those skilled in the art.

Compounds represented by formula (IA) according to the invention in which G2 is an oxygen atom, R2 is a hydrogen atom and R1 and R3, which are the same or different, represent a CO—R5 or CO—(CH₂)₂ₙ₊₁—X—R6 group, can be obtained in different ways.

According to a first embodiment, a molecule of glycerol is reacted with a compound corresponding to the formula A°-CO-A1 in which A1 is a reactive group selected for example in the group consisting of OH, Cl and OR″, R″ being an alkyl group, and A° is the R5 group or the (CH₂)₂ₙ₊₁—X—R6 group, possibly in the presence of coupling agents or activators known to those skilled in the art. Said reaction enables the synthesis of so-called symmetrical compounds, in which R1 and R3 have the same meaning. Said reaction can be carried out by adapting the protocols described for example in (Feuge, Gros et al. 1953), (Gangadhar, Subbarao et al. 1989), (Han, Cho et al. 1999) or (Robinson 1960).

Compounds represented by formula (IA) according to the invention in which G2 is an oxygen atom, R2 is a hydrogen atom and R1 and R3, which are the same or different, represent a CO—R5 or CO—(CH₂)₂ₙ₊₁—X—R6 group, can also be obtained from a compound represented by formula (IA) according to the invention in which G2 is an oxygen atom, R2 and R3 represent a hydrogen atom and R1 is a CO—R5 or CO—(CH₂)₂ₙ₊₁—X—R6 group (said particular form of formula (IA) compounds being named compounds (IV)), and a compound corresponding to the formula A+-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art. Advantageously, said reaction is carried out according to the protocol described for example in (Daubert, Spiegl et al. 1943), (Feuge and Lovegren 1956), (Katoch, Trivedi et al. 1999) or (Strawn, Martell et al. 1989).

Compounds (IV) described hereinabove can be prepared by a method comprising (diagram 1):
a) reacting a compound represented by formula (II) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (III) in which R1 represents a CO—R5 or CO—(CH₂)₂ₙ₊₁—X—R6 group;
b) deprotecting the compound (III) with an acid (acetic acid, trifluoroacetic acid, boric acid, sulfuric acid, etc.) to give a compound represented by general formula (IV) such as defined hereinabove.

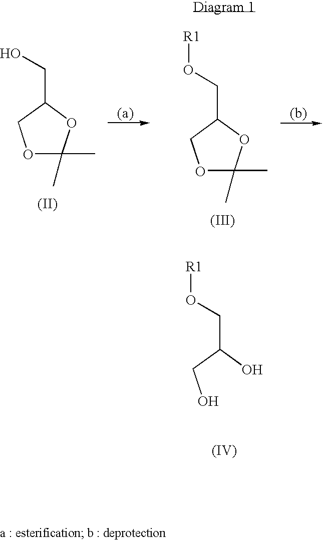

According to another particular method of the invention compounds represented by formula (IA) in which G2 is an oxygen atom, R3 is a hydrogen atom and R1 and R2, which are the same or different, represent a CO—R5 or CO—(CH₂)₂ₙ₊₁—X—R6 group, can be obtained from a compound represented by formula (IA) according to the invention in which G2 is an oxygen atom, R2 and R3 represent a hydrogen atom and R1 is a CO—R5 or CO—(CH₂)₂ₙ₊₁—X—R6 group (compounds IV), according to the following steps (diagram 2):
a) reacting the compound (IV) with a compound PxE in which Px is a protective group and E is a reactive group selected for example in the group consisting of OH and a halogen, to give a compound represented by general formula (V) in which R1 is a CO—R5 or CO—(CH₂)₂ₙ₊₁—X—R6 group. Advantageously, the reaction can be carried out by adapting the protocols described by (Gaffney and Reese 1997) in which PxE can represent the compound 9-phenylxanthene-9-ol or 9-chloro-9-phenylxanthene;
b) reacting the compound represented by formula (V) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (VI), in which R1 and R2, which are the same or different, represent a CO—R5 or CO—(CH₂)₂ₙ₊₁—X—R6 group and Px is a protective group;
c) deprotecting the compound (VI), in conventional conditions known to those skilled in the art to give a compound represented by general formula (IA) in which G2 is an oxygen atom, R3 is a hydrogen atom and R1 and R2, which are the same or different, represent a CO—R5 or CO—(CH₂)₂ₙ₊₁—X—R6 group.

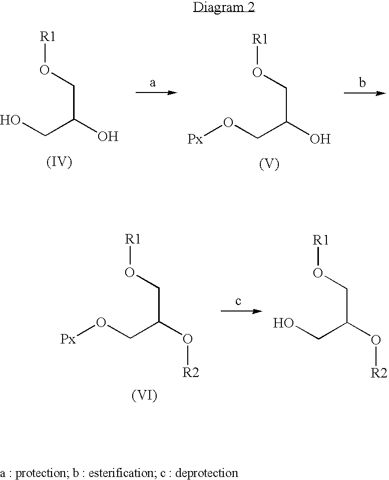

According to another particular method of the invention, compounds represented by general formula (IA) in which G2 is an oxygen atom, R1 and R3 represent a hydrogen atom and R2 represents a CO—R5 or CO—(CH₂)₂ₙ₊₁—X—R6 group, are obtained by a method comprising (diagram 3):
a) reacting a compound represented by formula (VII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (VIII) in which R2 represents a CO—R5 or CO—(CH₂)₂ₙ₊₁—X—R6 group;
b) deprotecting the compound having formula (VIII) in acidic medium or by catalytic hydrogenation to give a compound represented by general formula (IA) in which G2 is an oxygen atom, R1 and R3 represent a hydrogen atom and R2 represents a CO—R5 or CO—(CH₂)₂ₙ₊₁—X—R6 group.

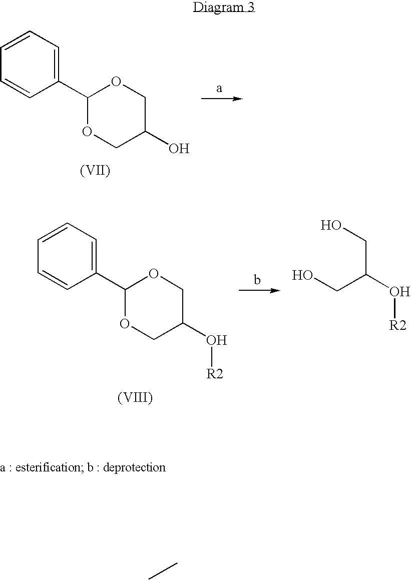

In an advantageous manner, the aforementioned steps can be carried out according to the protocols described by (Bodai, Novak et al. 1999), (Paris, Garmaise et al. 1980), (Scriba 1993) or (Seltzman, Fleming et al. 2000).

Compounds represented by formula (IA) according to the invention in which G2 is a sulfur atom, R2 is a hydrogen atom and R1 and R3, which are the same or different, represent a CO—R5 or CO—(CH₂)₂ₙ₊₁—X—R6 group, can be obtained from a compound having formula (IX) by the following method: 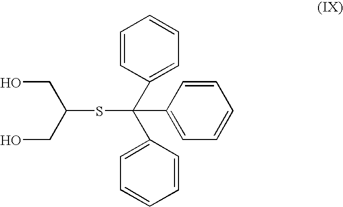
a) reacting the compound (IX) with a first compound corresponding to the formula A°-CO-A3 in which A3 is a reactive group selected for example in the group consisting of OH, O—CO-A° and Cl, and A° is the R5 group or the (CH₂)₂ₙ₊₁—X—R6 group, then with a second compound corresponding to the formula A°-CO-A3 in which, independently of the first compound, A3 is a reactive group selected for example in the group consisting of OH, O—CO-A+ and Cl, and A° is the R5 group or the (CH₂)₂ₙ₊₁—X—R6 group, possibly in the presence of coupling agents or activators known to those skilled in the art;
b) deprotecting the thiol group with mercuric acetate.

Advantageously, said method is carried out according to the protocol described by (Aveta, Brandt et al. 1986).

Compounds represented by formula (IA) according to the invention in which G2 is a sulfur atom, R2 and R3 are hydrogen atoms and R1 represents a CO—R5 or CO—(CH₂)₂ₙ₊₁—X—R6 group, can be obtained from the compound having formula (IX) by the following method:
a) reacting the compound (IX) with a first compound corresponding to the formula A°-CO-A3 in which A3 is a reactive group selected for example in the group consisting of OH, O—CO-A° and Cl, and A° is the R5 group or the (CH₂)₂ₙ₊₁—X—R6 group, in stoichiometric amounts, possibly in the presence of coupling agents or activators known to those skilled in the art;
b) deprotecting the thiol group with mercuric acetate.

The compound corresponding to formula (IX) can be prepared by a method comprising:
a) reacting a dimethyl 2-halogenomalonate with tritylthiol to give the compound represented by formula (X): 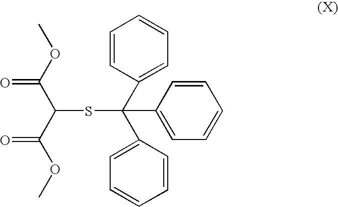
b) reducing the acetate functions with a reducing agent known to those skilled in the art.

Compounds represented by formula (IA) according to the invention in which G2 is a sulfur atom, and R1, R2 and R3, which are the same or different, represent a CO—R5 or CO—(CH₂)₂ₙ₊₁—X—R6 group, can also be obtained by the following method (diagram 4):
a) reacting a compound represented by formula (V) with a compound corresponding to the formula LG-E in which E represents a halogen and LG is a reactive group selected for example in the group consisting of mesyl, tosyl, etc., to give a compound represented by general formula (XI) in which Px represents a protective group;
b) reacting a compound represented by formula (XI) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XII). Advantageously, said reaction can be carried out by adapting the protocol described by (Gronowitz, Herslof et al. 1978);
c) deprotecting the sulfur atom of a compound (XII), in conditions known to those skilled in the art, to give a compound represented by general formula (XIII);
d) reacting a compound represented by general formula (XIII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XIV) in which R1 and R2, which are the same or different, represent a CO—R5 or CO—(CH₂)₂ₙ₊₁—X—R6 group;
e) deprotecting a compound represented by formula (XIV), in conventional conditions known to those skilled in the art, to give a compound represented by formula (IA) of the invention in which (i) G2 is a sulfur atom, (ii) R3 is a hydrogen atom and (iii) R1 and R2, which are the same or different, represent a CO—R5 or CO—(CH₂)₂ₙ₊₁—X—R6 group;
f) reacting a compound represented by formula (IA) according to the invention in which (i) G2 is a sulfur atom, (ii) R3 is a hydrogen atom and (iii) R1 and R2 represent a CO—R5 or CO—(CH₂)₂ₙ₊₁—X—R6 group, the same or different, with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

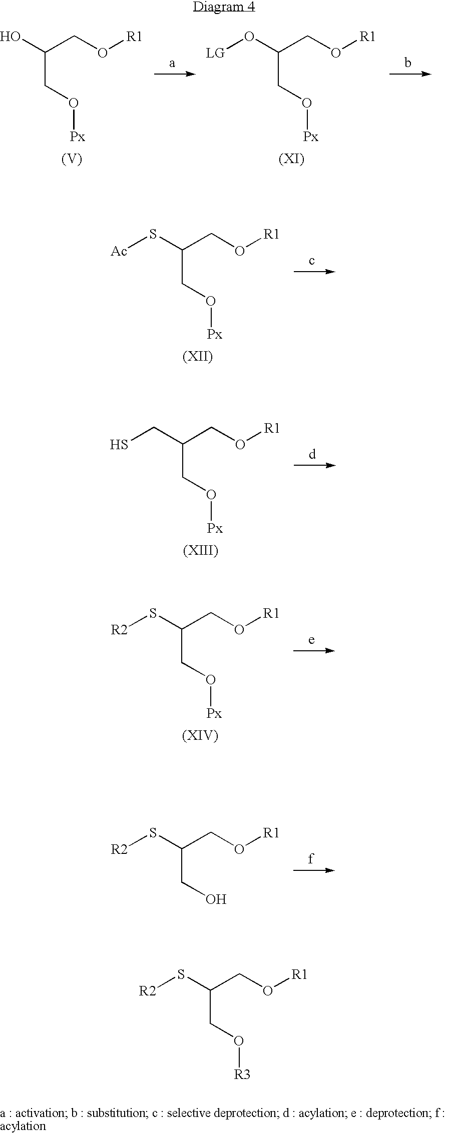

According to another embodiment, compounds represented by formula (IA) according to the invention in which G2 is a sulfur atom, and R1, R2 and R3, which are the same or different, represent a CO—R5 or CO—(CH₂)₂ₙ₊₁—X—R6 group, can also be obtained by the following method:
a) reacting a compound represented by general formula (IA) according to the invention in which (i) G2 is an oxygen atom (ii) R2 represents a hydrogen atom and (iii) R1 and R3, which are the same or different, represent a CO—R5 or CO—(CH₂)₂ₙ₊₁—X—R6 group such as defined hereinabove, with iodine in the presence of activating agents known to those skilled in the art to give a compound represented by formula (XV) in which R1 and R3, which are the same or different, represent a CO—R5 or CO—(CH₂)₂ₙ₊₁—X—R6 group; 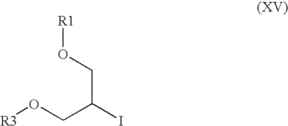
b) reacting a compound represented by formula (XV) with thiocarboxylic acid in the presence of coupling agents or activators known to those skilled in the art.

Compounds represented by formula (IA) in which G2 is a N—R4 group and in which R1, R2 and R3 which are the same or different, represent a CO—R5 group or a CO—(CH₂)₂ₙ₊₁—X—R6 group, are obtained from a compound represented by formula (IA) in which G2 is a N—R4 group, R1 and R3 are hydrogen atoms, R2 is a CO—R5 group or a CO—(CH₂)₂ₙ₊₁—X—R6 group (compound (XVI)) according to the following method: reacting a compound (XVI) with a first compound corresponding to the formula A°-CO-A2 in which A2 is a reactive group selected for example in the group consisting of OH and Cl, and A° is the R5 group or the (CH₂)₂ₙ₊₁—X—R6 group, then with a second compound corresponding to the formula A°-CO-A2 in which, independently of the first compound, A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Said method is advantageously carried out according to the protocol described by (Terradas 1993).

Compounds represented by formula (IA) in which G2 is a N—R4 group and in which R1 and R2 represent a CO—R5 group or a CO—(CH₂)₂ₙ₊₁—X—R6 group, and R3 is a hydrogen atom, can be obtained by reacting a compound (XVI) and a compound corresponding to the formula A°-CO-A2 in which A2 is a reactive group selected for example in the group consisting of OH and Cl, and A° is the R5 group or the (CH₂)₂ₙ₊₁—X—R6 group in stoichiometric amounts, possibly in the presence of coupling agents or activators known to those skilled in the art.

Compounds represented by formula (IA) according to the invention in which G2 is a NH group, R1 and R3 are hydrogen atoms, R2 is a CO—R5 group or a CO—(CH₂)₂ₙ₊₁—X—R6 group (compound XVIa) can be obtained in different ways. According to a first method, a molecule of 2-aminopropane-1,3-diol is reacted with a compound corresponding to the formula A°-CO-A in which A is a reactive group selected for example in the group consisting of OH, O—CO-A°, OR″ and Cl, and A° is the R5 group or the (CH₂)₂ₙ₊₁—X—R6 group possibly in the presence of coupling agents or activators known to those skilled in the art.

Said reaction can be carried out by adapting the protocols described for example in (Shaban 1977), (Kurfürst, Roig et al. 1993), (Harada, Morie et al. 1996), (Khanolkar, Abadji et al. 1996), (Daniher and Bashkin 1998) or (Putnam and Bashkin 2000).

Compounds represented by formula (IA) according to the invention in which G2 is a NH group, R1 and R3 are hydrogen atoms, R2 is a CO—R5 group or a CO—(CH₂)₂ₙ₊₁—X—R6 group (compound XVIa) can also be obtained according to the following method (diagram 5):
a) reacting a compound having formula (XVII) with a compound corresponding to the formula A°-CO-A in which A is a reactive group selected for example in the group consisting of OH, O—CO-A°, OR| and Cl and A° is the R5 group or the (CH₂)₂ₙ₊₁—X—R6 group possibly in the presence of coupling agents or activators known to those skilled in the art to give a compound represented by general formula (XVIII);
b) deprotecting the compound (XVIII).

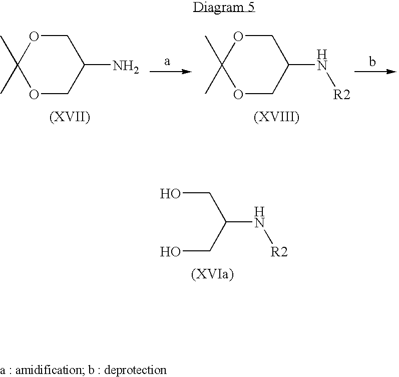

Advantageously, said method can be carried out according to the protocol described by (Harada, Morie et al. 1996).

Compounds represented by formula (IA) according to the invention in which G2 is a N—R4 group in which R4 is not a hydrogen atom, R1 and R3 are hydrogen atoms, R2 is a CO—R5 group or a CO—(CH₂)₂ₙ₊₁—X—R6 group (compound XVIb) can be obtained by the following method (diagram 6):
a) reacting a compound having formula (XVII) with a compound corresponding to the formula A°-CO-A in which A is a reactive group selected for example in the group consisting of OH, O—CO-A°, OR″ and Cl and A° is the R5 group or the (CH₂)₂ₙ₊₁—X—R6 group possibly in the presence of coupling agents or activators known to those skilled in the art to give a compound represented by general formula (XVIII);
b) reacting the compound (XVIII) either with a compound of the type R4-A4 in which A4 is a reactive group selected for example in the group consisting of Cl or Br, in basic medium, or with a R4CHO group in which CHO is the aldehyde function in the presence of reducing agents known to those skilled in the art, to give a compound (XIX);
c) deprotecting the compound (XIX).

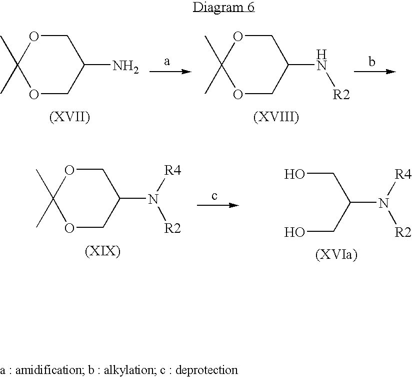

According to another method of the invention, compounds represented by formula (IB) in which (i) G2 and G3 are oxygen or sulfur atoms or a N—R4 group, (ii) R and, as the case may be, R4, represent an identical linear or branched alkyl group, saturated or not, optionally substituted, containing from 1 to 5 carbon atoms and (iii) R1, R2 and R3, which are the same or different, represent a CO—R5 group or a CO—(CH₂)₂ₙ₊₁—X—R6 group, are obtained from a compound represented by formula (IB) in which (i) G2 or G3 are oxygen or sulfur atoms or a NH group, (ii) R is a hydrogen atom and (iii) R1, R2 and R3, which are the same or different, represent a CO—R5 or CO—(CH₂)₂ₙ₊₁—X—R6 group, and a compound corresponding to the formula A1-LG in which Al represents the group R or, as the case may be, R4 and LG is a reactive group selected for example in the group consisting of Cl, Br, mesyl, tosyl, etc., possibly in the presence of coupling agents or activators known to those skilled in the art.

In a first embodiment, compounds represented by formula (IB) in which (i) G2 and G3 are oxygen or sulfur atoms or a NH group; (ii) R is a hydrogen atom and (iii) R1, R2 and R3, which are the same, represent a CO—(CH₂)₂ₙ₊₁—X—R6 group, are obtained from a compound represented by formula (IB) in which (i) G2 or G3 are oxygen or sulfur atoms or a NH group, (ii) R is a hydrogen atom and (iii) R1, R2 and R3 are hydrogen atoms and a compound corresponding to the formula A°-CO-A in which A is a reactive group selected for example in the group consisting of OH, Cl, O—CO-A° and O—R7, R7 being an alkyl group, and A° is the (CH₂)₂ₙ₊₁—X—R6 group, possibly in the presence of coupling agents or activators known to those skilled in the art.

Compounds represented by formula (IB) according to the invention in which (i) G2 and G3 are oxygen atoms or a NH group, (ii) R is a hydrogen atom and (iii) R1, R2 and R3 are hydrogen atoms or represent a CO—R5 or CO—(CH₂)₂ₙ₊₁—X—R6 group can be obtained by different methods which enable the synthesis of compounds in which the groups carried on a same heteroatom (nitrogen or oxygen) have the same meaning.

According to a first embodiment, a molecule of 1-aminoglycerol, 1,3-diaminoglycerol or 1,2-diaminoglycerol (obtained by adapting the protocol described by (Morris, Atassi et al. 1997)) is reacted with a compound corresponding to the formula A°-CO-A1 in which A1 is a reactive group selected for example in the group consisting of OH, Cl and OR7, R7 being an alkyl group, and A° is the R5 group or the (CH₂)₂ₙ₊₁—X—R6 group, possibly in the presence of coupling agents or activators known to those skilled in the art. Said reaction respectively yields particular forms of compounds represented by formula (IB), named compounds (XXa-c), and can be carried out by adapting the protocols described by (Urakami and Kakeda 1953), (Shealy, Frye et al. 1984), (Marx, Piantadosi et al. 1988), (Rahman, Ziering et al. 1988) and (Nazih, Cordier et al. 1999). In compounds (XXb-c), the groups carried on a same heteroatom, respectively, (R1 and R3) and (R1 and R2) have the same meaning.

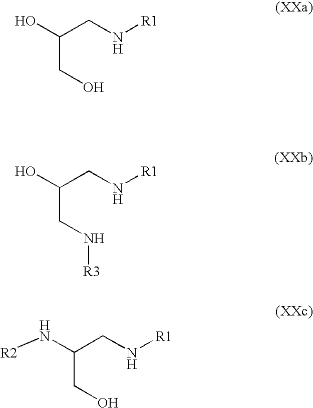

Compounds represented by formula (IB) according to the invention in which (i) G2 and G3 are oxygen atoms or a NH group, (ii) R is a hydrogen atom and (iii) R1, R2 and R3, which are the same or different, represent a CO—R5 or CO—(CH₂)₂ₙ₊₁—X—R6 group, can be obtained from a compound having formula (XXa-c) and a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art. Said reaction enables the synthesis of compounds in which the groups carried on a same heteroatom (nitrogen or oxygen), respectively (R1 and R2), (R1 and R3) or (R2 and R3) have the same meaning. Advantageously, said reaction is carried out according to the protocol described for example in (Urakami and Kakeda 1953) and (Nazih, Cordier et al. 1999).

According to another particular method of the invention, compounds represented by formula (IB) in which (i) G2 and G3 are oxygen atoms or a NH group (ii) R is a hydrogen atom and (iii) R1, R2 and R3, which are the same or different, represent a CO—R5 or CO—(CH₂)₂ₙ₊₁—X—R5 group, can be obtained according to the following steps (diagram 7):
a) reacting 1-aminoglycerol, 1,3-diaminoglycerol or 1,2-diaminoglycerol with a compound (PG)₂O in which PG is a protective group to give a compound having general formula (XXIa-c). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
b) reacting the compound having formula (XXIa-c) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXIIa-c), in which R2 and R3 represent a CO—R5 or CO—(CH₂)₂ₙ₊₁—X—R6 group and PG is a protective group;
c) deprotecting the compound (XXIIa-c), according to conventional conditions known to those skilled in the art, to give a compound represented by general formula (IB) in which (i) G2 and G3 represent an oxygen atom or a NH group, (ii) R and R1 are hydrogen atoms and (iii) R2 and R3 represent a hydrogen atom or a CO—R5 or CO—(CH₂)₂ₙ₊₁—X—R6 group;
d) reacting a compound represented by general formula (IB) in which (i) G2 and G3 represent an oxygen atom or a NH group, (ii) R and R1 are hydrogen atoms and (iii) R2 and R3 represent a hydrogen atom or a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

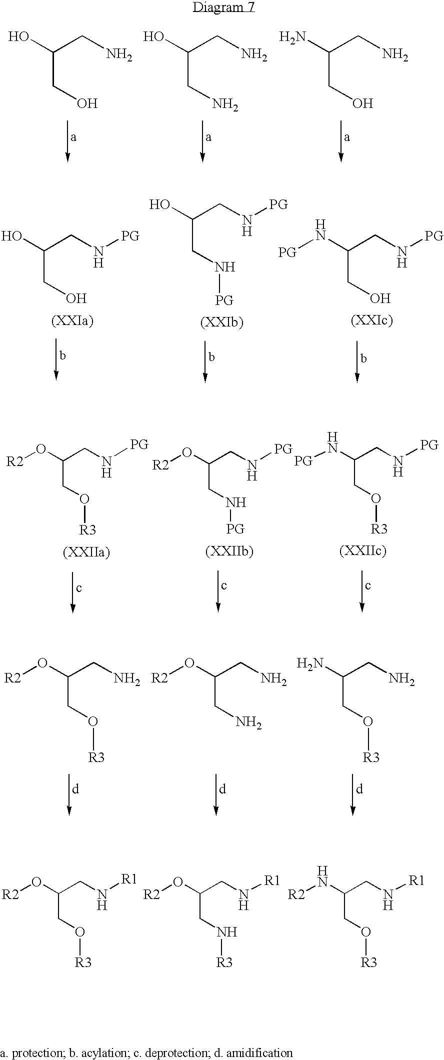

Compounds represented by formula (IB) according to the invention in which (i) G2 and G3 are oxygen atoms, (ii) R is a hydrogen atom and (iii) R1, R2 and R3, which are the same or different, represent a CO—R5 or CO—(CH₂)₂ₙ₊₁—X—R6 group, can be obtained in different ways.

According to a first method, a compound represented by formula (IB) according to the invention, in which (i) G2 and G3 are oxygen atoms, (ii) R and R2 are hydrogen atoms and (iii) R1 and R3, which are the same or different, represent a CO—R5 or CO—(CH₂)₂ₙ₊₁—X—R6 group, is reacted with a compound corresponding to the formula A°-CO-A2 in which A2 a reactive group selected for example in the group consisting of OH and Cl, and A° is the R5 group or the (CH₂)₂ₙ₊₁—X—R6 group, possibly in the presence of coupling agents or activators known to those skilled in the art.

According to this method of preparation, compounds represented by formula (IB) in which (i) G2 and G3 are oxygen atoms, (ii) R and R2 are hydrogen atoms and (iii) R1 and R3, which are the same or different, represent a CO—R5 or CO—(CH₂)₂ₙ₊₁—X—R6 group, can be obtained from a compound represented by formula (XXa) such as defined hereinabove and a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

According to another particular inventive method, compounds represented by formula (IB) in which (i) G2 and G3 are oxygen atoms, (ii) R is a hydrogen atom and (iii) R1, R2 and R3, which are the same or different, represent a CO—R5 or CO—(CH₂)₂ₙ₊₁—X—R6 group, can be obtained from a compound represented by formula (IB) according to the invention in which (i) G2 and G3 are oxygen atoms, (ii) R, R2 and R3 represent a hydrogen atom and (iii) R1 is a CO—R5 or CO—(CH₂)₂ₙ₊₁—X—R6 group (compound having formula (XXa)) according to the following steps (diagram 8):
a) reacting a compound represented by formula (XXa) with a compound PG-E in which PG is a protective group and E is a reactive group selected for example in the group consisting of OH and a halogen, to give a compound represented by general formula (XXIII) in which R1 is a CO—R5 or CO—(CH₂)₂ₙ₊₁—X—R6 group. Advantageously, the reaction can be carried out by adapting the protocols described by (Marx, Piantadosi et al. 1988) and (Gaffney and Reese 1997) in which PG-E can represent triphenylmethyl chloride or 9-phenylxanthene-9-ol or else 9-chloro-9-phenylxanthene;
b) reacting the compound represented by formula (XXIII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXIV), in which R1 and R2, which are the same or different, represent a CO—R5 or CO—(CH₂)₂ₙ₊₁—X—R6 group and PG is a protective group;
c) deprotecting the compound (XXIV), in conditions known to those skilled in the art, to give a compound represented by general formula (IB) in which (i) G2 and G3 are oxygen atoms, (ii) R and R3 are hydrogen atoms and (iii) R1 and R2, which are the same or different, represent a CO—R5 or CO—(CH₂)₂ₙ₊₁—X—R6 group;
d) reacting a compound represented by general formula (IB) in which (i) G2 and G3 are oxygen atoms, (ii) R and R3 are hydrogen atoms and (iii) R1 and R2, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

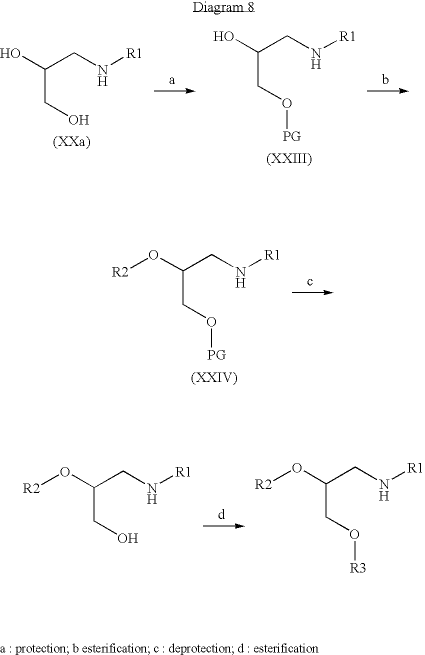

In an advantageous manner, the hereinabove steps are carried out according to the protocols described by (Marx, Piantadosi et al. 1988).

According to another method of the invention, compounds represented by formula (IB) in which (i) G2 or G3 represent an oxygen atom or a N—R4 group, (ii) at least one of the groups G2 or G3 represents a N—R4 group, (iii) R and R4 independently represent linear or branched alkyl groups, saturated or not, optionally substituted, containing from 1 to 5 carbon atoms and (iv) R1, R2 and R3, which are the same or different, represent a CO—R5 group or a CO—(CH₂)₂ₙ₊₁—X—R6 group, are obtained by reacting a compound represented by formula (IB) in which (i) one of the groups G2R2 or G3R3 represents a hydroxyl group and the other group G2R2 or G3R3 represents a NR4R2 or NR4R3 group, respectively, with R2 or R3 representing a CO-R5 group or a CO—(CH₂)₂ₙ₊₁—X—R6 group, (ii) R and R4 independently represent a linear or branched alkyl group, saturated or not, optionally substituted, containing from 1 to 5 carbon atoms and (iii) R1 represents a CO—R5 group or a CO—(CH₂)₂ₙ₊₁—X—R6 group, with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Compounds represented by formula (IB) according to the invention in which (i) one of the groups G2R2 or G3R3 represents a hydroxyl group and the other group G2R2 or G3R3 represents a NR4R2 or NR4R3 group, respectively, with R2 or R3 representing a CO—R5 group or a CO—(CH₂)₂ₙ₊₁—X—R6 group, (ii) R and R4 independently represent linear or branched alkyl groups, saturated or not, optionally substituted, containing from 1 to 5 carbon atoms and (iii) R1 represents a CO—R5 group or a CO—(CH₂)₂ₙ₊₁—X—R6 group, are obtained from a compound represented by formula (IB) according to the invention in which one of the groups G2R2 or G3R3 represents a hydroxyl group and the other group G2R2 or G3R3 represents a NR4R2 or NR4R3 group, respectively, with R2 or R3 representing a CO—R5 group or a CO—(CH₂)₂ₙ₊₁—X—R6 group, (ii) R and R4 independently represent a group such as defined hereinabove and (iii) R1 is a hydrogen atom with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

In a first embodiment, compounds represented by formula (IB) according to the invention in which (i) G2 is an oxygen atom, (ii) G3 represents a N—R4 group, (iii) R and R4 independently represent different linear or branched alkyl groups, saturated or not, optionally substituted, containing from 1 to 5 carbon atoms, (iv) R1 and R2 are hydrogen atoms and (v) R3 represents a CO—R5 group or a CO—(CH₂)₂ₙ₊₁—X—R6 group are obtained in the following manner (diagram 9):
a) reacting 1-aminoglycerol with a compound corresponding to the formula R—CHO in which R represents a linear or branched alkyl group, saturated or not, optionally substituted, containing from 1 to 5 carbon atoms and CHO is the aldehyde function in the presence of reducing agents known to those skilled in the art to give a compound represented by formula (XXV) in which R is a group such as defined hereinabove. Advantageously, said reaction can be carried out by adapting the protocols described by (Antoniadou-Vyzas, Foscolos et al. 1986);
b) reacting a compound represented by formula (XXV) with a compound (PG)₂O in which PG is a protective group to give a compound represented by general formula (XXVI). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
c) reacting a compound represented by formula (XXVI) with a compound corresponding to the formula LG-E in which E represents a halogen and LG is a reactive group selected for example in the group consisting of mesyl, tosyl, etc., to give a compound represented by general formula (XXVII) by adapting the method described by (Kitchin, Bethell et al. 1994);
d) reacting a compound represented by formula (XXVII) with a compound corresponding to the formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to give a compound corresponding to formula (XXVIII) in which R and R4, optionally different, are such as defined hereinabove;
e) reacting a compound represented by formula (XXVIII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (XXIX) in which R and R4 represent different linear or branched alkyl groups, saturated or not, optionally substituted, containing from 1 to 5 carbon atoms, R3 represents the R5 group or the (CH₂)₂ₙ₊₁—X—R6 group and PG is a protective group;
f) deprotecting the compound (XXIX) in conditions known to those skilled in the art.

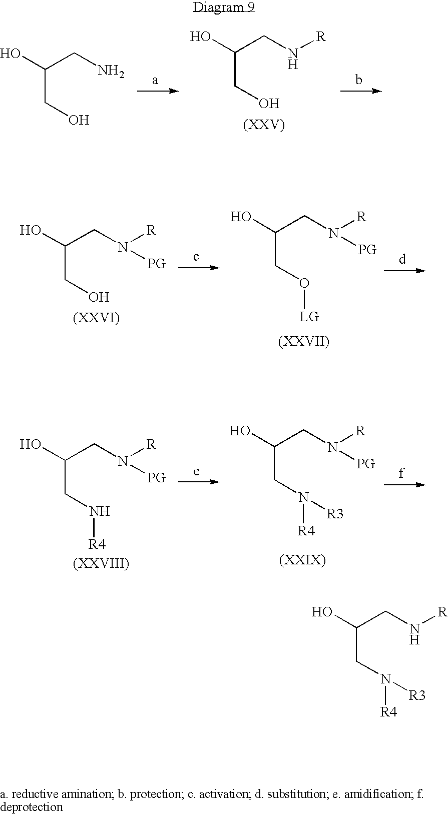

According to a second embodiment, compounds represented by formula (IB) according to the invention in which (i) G3 is an oxygen atom, (ii) G2 represents a N—R4 group, (iii) R and R4 represent different linear or branched alkyl groups, saturated or not, optionally substituted, containing from 1 to 5 carbon atoms, (iv) R1 and R3 are hydrogen atoms and (v) R2 represents a CO—R5 group or a CO—(CH₂)₂ₙ₊₁—X—R6 group are obtained in the following manner (diagram 10):
a) reacting a compound represented by formula (XXVI) with a compound PG′-E in which PG′ is a protective group and E is a reactive group selected for example in the group consisting of OH and a halogen, to give a compound represented by general formula (XXX) in which R represents a linear or branched alkyl group, saturated or not, optionally substituted, containing from 1 to 5 carbon atoms and PG is another protective group such as defined hereinabove. Advantageously, the reaction can be carried out by adapting the protocols described by (Marx, Piantadosi et al. 1988) and (Gaffney and Reese 1997) in which PG′-E can represent triphenylmethyl chloride or 9-phenylxanthene-9-ol or else 9-chloro-9-phenylxanthene;
b) reacting a compound represented by formula (XXX) such as defined hereinabove with a compound corresponding to the formula LG-E in which E represents a halogen and LG is a reactive group selected for example in the group consisting of mesyl, tosyl, etc., to give a compound represented by general formula (XXXI) in which R represents a linear or branched alkyl group, saturated or not, optionally substituted, containing from 1 to 5 carbon atoms and PG and PG′ are protective groups, by adapting the method described by (Kitchin, Bethell et al. 1994);
c) reacting a compound represented by formula (XXXI) such as defined hereinabove with a compound corresponding to the formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to obtain a compound represented by formula (XXXII) in which R and R4 are independently such as defined hereinabove;
d) reacting a compound represented by formula (XXXII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (XXXIII) in which R and R4 independently represent linear or branched alkyl groups, saturated or not, optionally substituted, containing from 1 to 5 carbon atoms, R2 represents a CO—R5 group or a CO—(CH₂)₂ₙ₊₁—X—R6 group, PG and PG′ are protective groups;
e) deprotecting a compound represented by formula (XXXIII) in conventional conditions known to those skilled in the art to obtain a compound represented by general formula (IB) according to the invention in which (i) R and R4 independently represent linear or branched alkyl groups, saturated or not, optionally substituted, containing from 1 to 5 carbon atoms, (ii) R1 and R3 are hydrogen atoms and (iii) R2 represents a CO—R5 group or a CO—(CH₂)₂ₙ₊₁—X—R6 group.

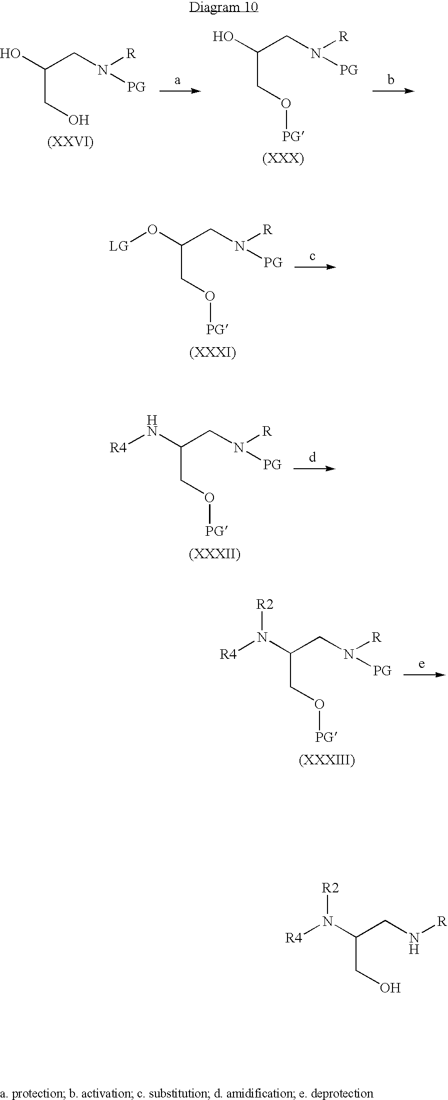

Compounds represented by formula (IB) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group can be obtained by different methods.

According to a first embodiment, compounds represented by formula (IB) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group, R1, R2 and/or R3 having the same meaning when they are carried on a same heteroatom (sulfur or nitrogen), can be obtained in the following manner (diagram 11A):
a) reacting a compound represented by formula (XXa-c) with a compound corresponding to the formula LG-E in which E represents a halogen and LG is a reactive group selected for example in the group consisting of mesyl, tosyl, etc., to give a compound represented by general formula (XXXIVa-c);
b) reacting a compound represented by formula (XXXIVa-c) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XXXVa-c). Advantageously, said reaction can be carried out by adapting the protocol described by (Gronowitz, Herslöf et al. 1978);
c) deprotecting a compound represented by formula (XXXVa-c), in conventional conditions known to those skilled in the art, and for example in basic medium, to give a compound represented by general formula (IB) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group;
d) reacting a compound represented by general formula (IB) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group, with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

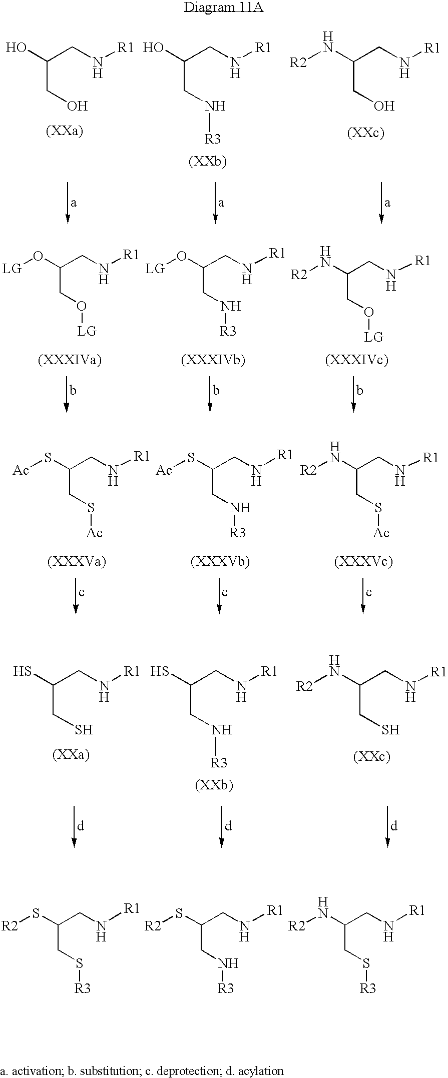

According to a similar synthetic method, compounds having formula (IB) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group, R1, R2 and/or R3 having the same meaning when they are carried on a same heteroatom (sulfur or nitrogen), can be prepared in the following manner (diagram 11B):
a) reacting a compound represented by formula (XXa-c) with a compound corresponding to the formula (LG)2 in which LG is a reactive group selected for example in the group consisting of iodine, bromine, etc., possibly in the presence of activators known to those skilled in the art to give a compound represented by general formula (XXXIVd-f);
b) reacting a compound represented by formula (XXXIVd-f) with a compound corresponding to the formula HS⁻B⁺ in which B is a counter-ion selected for example in the group consisting of sodium or potassium, preferably sodium to give a compound represented by general formula (IB) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group;
c) reacting a compound represented by general formula (IB) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group, with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

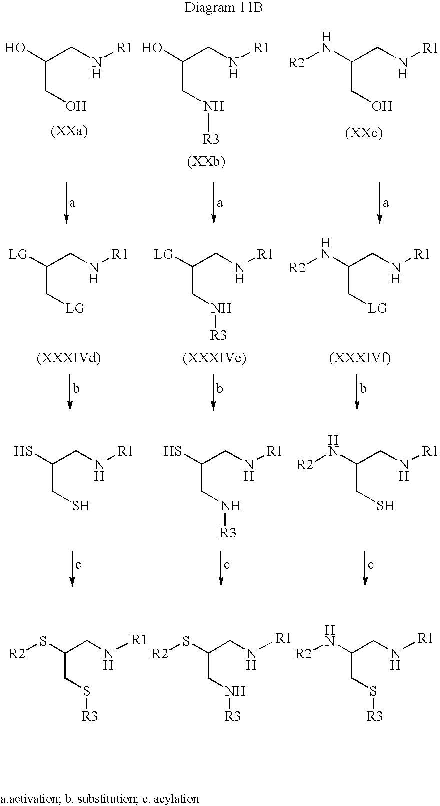

Said reaction enables the synthesis of compounds represented by general formula (IB) in which the groups carried on a same heteroatom (nitrogen or sulfur) respectively (R2 and R3), (R1 and R3) and (R1 and R2) have the same meaning.

The above steps can be carried out in an advantageous manner according to the protocols described by (Adams, Doyle et al. 1960) and (Gronowitz, Herslöf et al. 1978).

According to another method of the invention, compounds represented by formula (IB) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group can be prepared from compounds represented by formula (XXIa-c) by a method comprising (diagram 12):
a) reacting a compound represented by formula (XXIa-c) with a compound corresponding to the formula LG-E in which E represents a halogen and LG is a reactive group selected for example in the group consisting of mesyl, tosyl, etc., to give a compound represented by general formula (XXXVIa-c) in which PG represents a protective group;
b) reacting a compound represented by formula (XXXVIa-c) with a compound corresponding to the formula Ac—S⁻B⁺ in which Ac represents a short acyl group, preferably the acetyl group, and B is a counter-ion selected for example in the group consisting of sodium and potassium, preferably potassium to give a compound represented by general formula (XXXVIIa-c). Advantageously, said reaction can be carried out by adapting the protocol described by (Gronowitz, Herslöf et al. 1978);
c) deprotecting the sulfur atom of a compound (XXXVIIa-c) in conditions known to those skilled in the art, to give a compound represented by general formula (XXXVIIIa-c);
d) reacting a compound represented by general formula (XXXVIIIa-c) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXXIXa-c) in which R2 and R3 represent a CO—R5 or CO—(CH₂)₂ₙ₊₁—X—R6 group;
e) deprotecting a compound represented by formula (XXXIXa-c) in conventional conditions known to those skilled in the art, to give a compound represented by formula (IB) according to the invention in which (i) G2 and G3 are sulfur atoms or a NH group, (ii) R and R1 are hydrogen atoms and (iii) R2 and R3 represent a hydrogen atom, a CO—R5 or CO—(CH₂)₂ₙ₊₁—X—R6 group;
f) reacting a compound represented by formula (IB) according to the invention in which (i) G2 and G3 are sulfur atoms or a NH group, (ii) R and R1 are hydrogen atoms and (iii) R2 and R3 represent a hydrogen atom, a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Said reaction enables the synthesis of compounds represented by general formula (IB) in which the groups carried on a same heteroatom (nitrogen or sulfur) respectively (R2 and R3), (R1 and R3) and (R1 and R2) have the same meaning.

Advantageously, the above steps can be carried out according to the protocols described by (Adams, Doyle et al. 1960), (Gronowitz, Herslöf et al. 1978), (Bhatia and Hajdu 1987) and (Murata, Ikoma et al. 1991).

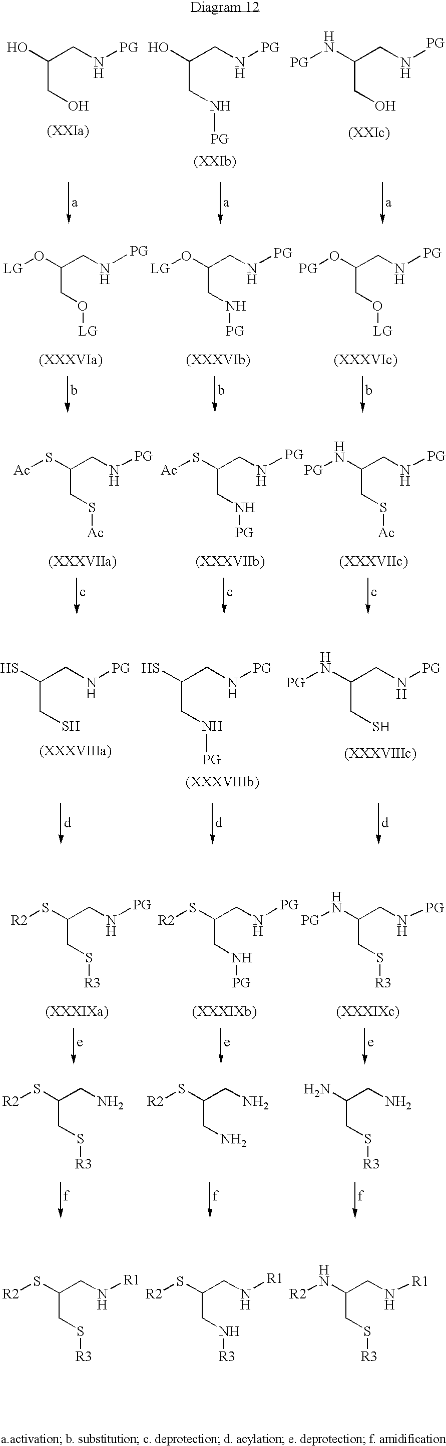

Compounds represented by general formula (IB) in which (i) G2 or G3 represent a sulfur atom or a N—R4 group, (ii) R and R4 independently represent a linear or branched alkyl group, saturated or not, optionally substituted, containing from 1 to 5 carbon atoms, (iii) R1, R2 and R3, which are the same or different, represent a CO—R5 group or a CO—(CH₂)₂ₙ₊₁—X—R6 group, are obtained by reacting a compound represented by general formula (IB) in which (i) G2 or G3 represent a sulfur atom or a N—R4 group, (ii) R and R4 independently represent groups such as defined hereinabove, (iii) R1 is a hydrogen atom and (iv) R2 and R3, which are the same or different, represent a CO—R5 group or a CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Compounds represented by general formula (IB) in which (i) the groups G2 and G3 represent a sulfur atom or a N—R4 group, (ii) R and R4 independently represent groups such as defined hereinabove, (iii) R1 is a hydrogen atom and (iv) R2 and R3, which are the same or different, represent a CO—R5 group or a CO—(CH₂)₂ₙ₊₁—X—R6 group, can be obtained by the following methods:

In a first embodiment, compounds represented by formula (IB) according to the invention in which (i) the group G2 is a sulfur atom, (ii) G3 represents a N—R4 group, (iii) R and R4 independently represent different linear or branched alkyl group, saturated or not, optionally substituted, containing from 1 to 5 carbon atoms, (iv) R1 is a hydrogen atom and (v) R2 and R3, which are the same or different, represent a CO—R5 group or a CO—(CH₂)₂ₙ₊₁—X—R6 group are obtained in the following manner (diagram 13):
a) reacting a compound represented by formula (XXIX) with a compound corresponding to the formula LG-E in which E represents a halogen and LG is a reactive group selected for example in the group consisting of mesyl, tosyl, etc., to give a compound represented by general formula (XL) in which PG represents a protective group;
b) reacting a compound represented by formula (XL) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XLI). Advantageously, said reaction is carried out by adapting the protocol described by (Gronowitz, Herslöf et al. 1978);
c) deprotecting the sulfur atom of a compound represented by formula (XLI) in conventional conditions known to those skilled in the art to give a compound represented by general formula (XLII);
d) reacting a compound represented by general formula (XLII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XLIII) in which R2 and R3, which are the same or different, represent a CO—R5 or CO—(CH₂)₂ₙ₊₁—X—R6 group;
e) deprotectig the compound of formula (XLIII) in conditions known to those skilled in the art.

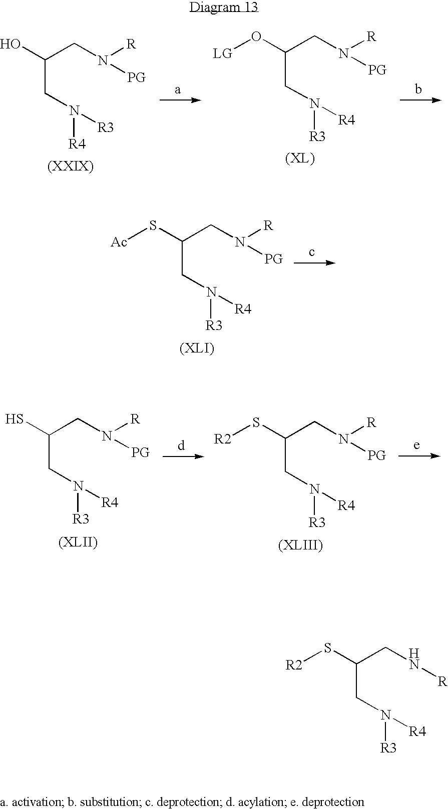

According to another method, compounds represented by formula (IB) according to the invention in which (i) G2 represents a N—R4 group, (ii) G3 is a sulfur atom, (iii) R and R4 independently represent different linear or branched alkyl groups, saturated or not, optionally substituted, containing from 1 to 5 carbon atoms, (iv) R1 is a hydrogen atom and (v) R2 and R3, which are the same or different, represent a CO—R5 group or a CO—(CH₂)₂ₙ₊₁—X—R6 group are obtained in the following manner (diagram 14):
a) reacting the compound represented by formula (XXVII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XLIV). Advantageously, said reaction can be carried out by adapting the protocol described by (Gronowitz, Herslöf et al. 1978);
b) reacting a compound represented by formula (XLIV) with a compound corresponding to the formula LG-E in which E represents a halogen and LG is a reactive group selected for example in the group consisting of mesyl, tosyl, etc., to give a compound represented by general formula (XLV) in which PG represents a protective group;
c) reacting the compound (XLV) with a compound represented by formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to give a compound represented by formula (XLVI) in which R and R4 independently represent different linear or branched alkyl groups, saturated or not, optionally substituted, containing from 1 to 5 carbon atoms;
d) reacting a compound represented by general formula (XLVI) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XLVII);
e) deprotecting the sulfur atom of a compound represented by formula (XLVII) in conventional conditions known to those skilled in the art to give a compound represented by general formula (XLVIII);
f) reacting a compound represented by general formula (XLVIII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XLIX) in which R2 and R3, which are the same or different, represent a CO—R5 or CO—(CH₂)₂ₙ₊₁—X—R6 group;
g) deprotecting a compound represented by formula (XLIX) in conventional conditions known to those skilled in the art.

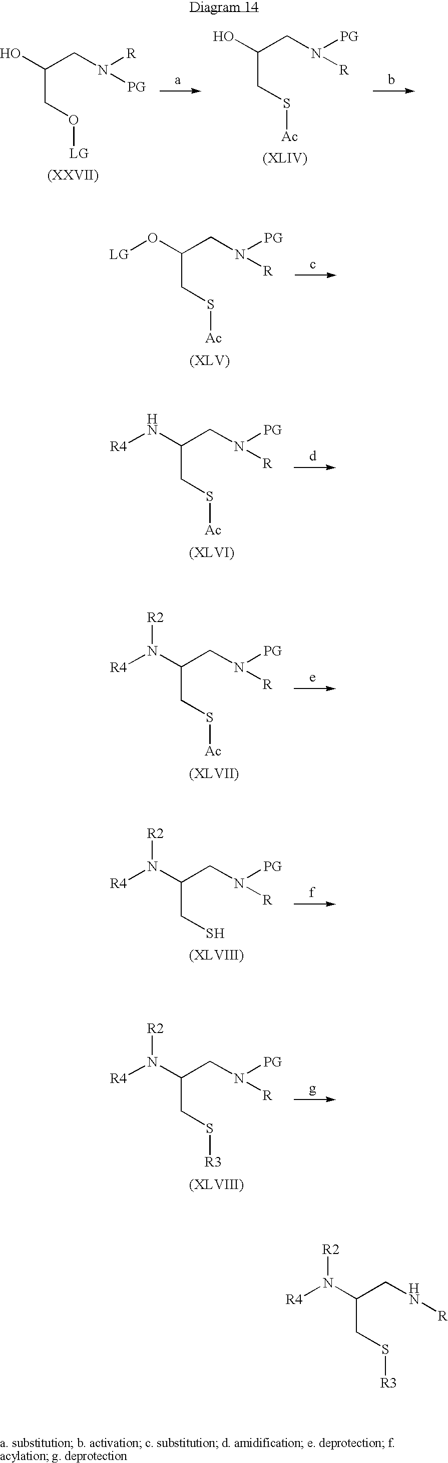

Compounds represented by formula (IB) according to the invention in which (i) G2 is a sulfur atom, (ii) G3 is an oxygen atom, (iii) R is a hydrogen atom, (iv) R1 and R2 represent a CO—R5 or CO—(CH₂)₂ₙ₊₁—X—R6 group and (v) R3 is a hydrogen atom or represents a CO—R5 or CO—(CH₂)₂ₙ₊₁—X—R6 group, can be prepared from compounds having formula (XXIII) according to the following method (diagram 15A):
a) reacting the compound (XXIII) with a compound corresponding to the formula LG-E in which E represents a halogen and LG is a reactive group selected for example in the group consisting of mesyl, tosyl, etc., to give a compound represented by general formula (L) in which PG represents a protective group;
b) reacting a compound represented by formula (L) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (LI). Advantageously, said reaction can be carried out by adapting the protocol described by (Gronowitz, Herslöf et al. 1978);
c) deprotecting the sulfur atom of a compound (LI), in conventional conditions known to those skilled in the art, to give a compound represented by general formula (LII);
d) reacting a compound represented by general formula (LII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LIII) in which R1 and R2, which are the same or different, represent a CO—R5 or CO—(CH₂)₂ₙ₊₁—X—R6 group;
e) deprotecting a compound (LIII) in conventional conditions known to those skilled in the art to give a compound represented by general formula (IB) in which G2 is a sulfur atom, G3 is an oxygen atom, R and R3 are hydrogen atoms and R1 and R2, which are the same or different, represent a CO—R5 or CO—(CH₂)₂ₙ₊₁—X—R6 group;
f) reacting a compound represented by general formula (IB) in which (i) G2 is a sulfur atom, (ii) G3 is an oxygen atom, (iii) R and R3 are hydrogen atoms and (iv) R1 and R2, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

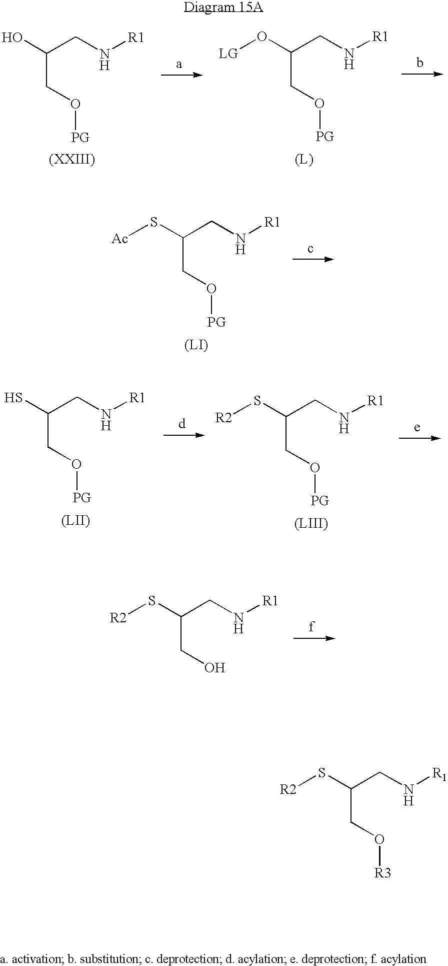

According to a similar method of synthesis, compounds represented by formula (IB) according to the invention in which (i) G2 is a sulfur atom, (ii) G3 is an oxygen atom, (iii) R is a hydrogen atom, (iv) R1 and R2 represent a CO—R5 or CO—(CH₂)₂ₙ₊₁—X—R6 group and (v) R3 is a hydrogen atom or represents a CO—R5 or CO—(CH2)₂ₙ₊₁—X—R6 group, can be prepared from compounds having formula (XXIII) by the following method (diagram 15B):
a) reacting the compound (XXIII) with a compound corresponding to the formula (LG)₂ in which LG is a reactive group selected for example in the group consisting of iodine, bromine, etc., to give a compound represented by general formula (La) in which PG represents a protective group;
b) reacting a compound represented by formula (La) with a compound corresponding to the formula HS⁻B⁺ in which B is a counter-ion selected for example in the group consisting of sodium and potassium, preferably sodium to give a compound represented by general formula (LII);
c) reacting a compound represented by general formula (LII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LIII) in which R1 and R2, which are the same or different, represent a CO—R5 or CO—(CH₂)₂ₙ₊₁—X—R6 group;
d) deprotecting the compound (LIII) in conventional conditions known to those skilled in the art to give a compound represented by general formula (IB) in which G2 is a sulfur atom, G3 is an oxygen atom, R and R3 are hydrogen atoms and R1 and R2, which are the same or different, represent a CO—R5 or CO—(CH₂)₂ₙ₊₁—X—R6 group;
e) reacting a compound represented by general formula (IB) in which (i) G2 is a sulfur atom, (ii) G3 is an oxygen atom, (iii) R and R3 are hydrogen atoms and (iv) R1 and R2, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

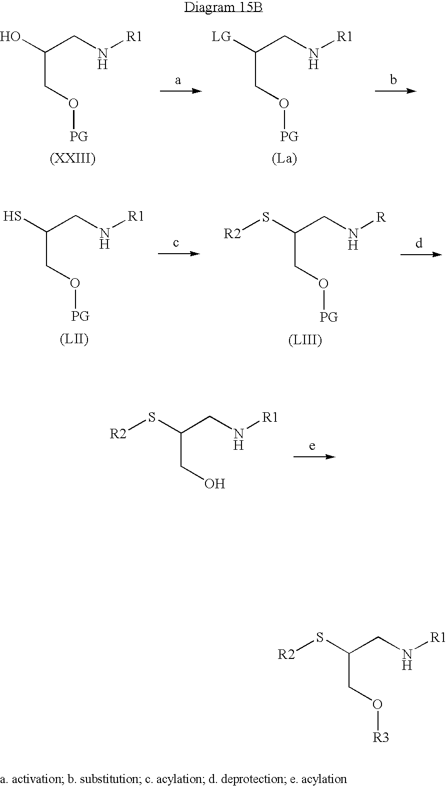

Compounds represented by formula (IB) according to the invention in which (i) G2 is a sulfur atom, (ii) G3 is an oxygen atom, (iii) R is a hydrogen atom, (iv) R1 and R3 represent a hydrogen atom or a CO—R5 or CO—(CH₂)₂ₙ₊₁—X—R6 group, which are the same or different, and (v) R2 represents a CO—R5 or CO—(CH₂)₂ₙ₊₁—X—R6 group, can be prepared from compounds having formula (XXIa) by the following method (diagram 16):
a) reacting a compound represented by formula (XXIa) with a compound PG′-E in which PG′ is a protective group and E is a reactive group selected for example in the group consisting of OH and a halogen, to give a compound represented by general formula (LIV) in which PG is another protective group such as defined earlier. In an advantageous manner, the reaction can be carried out by adapting the protocols described by (Marx, Piantadosi et al. 1988) and (Gaffney and Reese 1997) in which PG-E can represent triphenylmethyl chloride or 9-phenylxanthene-9-ol or else 9-chloro-9-phenylxanthene;
b) reacting the compound (LIV) with a compound corresponding to the formula LG-E in which E represents a halogen and LG is a reactive group selected for example in the group consisting of mesyl, tosyl, etc., to give a compound represented by general formula (LV) in which PG and PG′ represent judiciously selected protective groups such as defined hereinabove;
c) reacting a compound represented by formula (LV) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (LVI). Advantageously, said reaction can be carried out by adapting the protocol described by (Gronowitz, Herslöf et al. 1978);
d) deprotecting the sulfur atom of a compound (LVI), in conventional conditions known to those skilled in the art, to give a compound represented by general formula (LVII);
e) reacting a compound represented by general formula (LVII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LVIII) in which R2 represents a CO—R5 or CO—(CH₂)₂ₙ₊₁—X—R6 group;
f) deprotecting a compound (LVIII) in conventional conditions known to those skilled in the art to give a compound represented by general formula (IB) in which G2 is a sulfur atom, G3 is an oxygen atom, R, R1 and R3 are hydrogen atoms and R2 represents a CO—R5 or CO—(CH₂)₂ₙ₊₁—X—R6 group (compound LIX);
g) reacting a compound represented by formula (LIX) with a compound (PG)₂O in which PG is a protective group to give a compound represented by general formula (LX). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
h) reacting a compound represented by general formula (LX) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (LXI);
i) deprotecting a compound (LXI) in conventional conditions known to those skilled in the art to give a compound represented by general formula (IB) in which G2 is a sulfur atom, G3 is an oxygen atom, R and R1 are hydrogen atoms and R2 and R3, which are the same or different, represent a CO—R5 or CO—(CH₂)₂ₙ₊₁—X—R6 group;
j) reacting a compound represented by general formula (IB) in which G2 is a sulfur atom, G3 is an oxygen atom, R and R1 are hydrogen atoms and R2 and R3, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

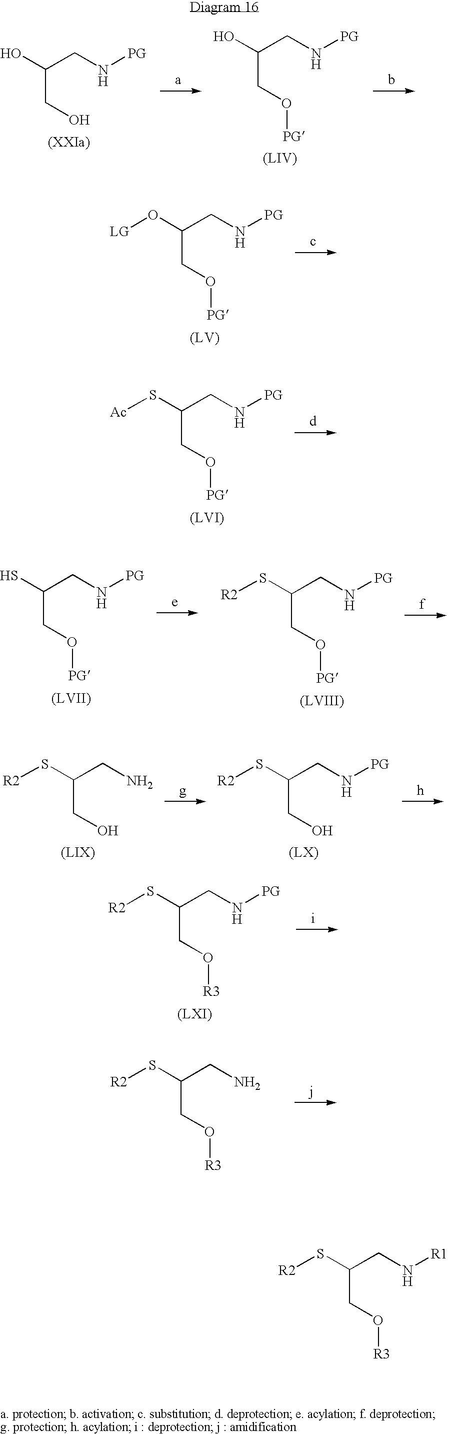

Compounds represented by formula (IB) according to the invention in which (i) G2 is an oxygen atom, (ii) G3 is a sulfur atom, (iii) R is a hydrogen atom, (iv) R2 is a hydrogen atom or represents a CO—R5 or CO—(CH₂)₂ₙ₊₁—X—R6 group and (v) R1 and R3 represent a CO—R5 or CO—(CH₂)₂ₙ₊₁—X—R6 group, can be prepared from compounds having formula (XXa) according to the following method (diagram 17):
a) reacting a compound represented by formula (XXa) such as defined hereinabove, with a compound corresponding to the formula LG-E (in stoichiometric amounts) in which E represents a halogen and LG is a reactive group selected for example in the group consisting of mesyl, tosyl, etc., to give a compound represented by general formula (LXII);
b) reacting a compound represented by formula (LXII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (LXIII). Advantageously, said reaction can be carried out by adapting the protocol described by (Gronowitz, Herslöf et al. 1978);
c) reacting a compound represented by formula (LXIII) with a compound PG-E in which PG is a protective group and E is a reactive group selected for example in the group consisting of OH and a halogen, to give a compound represented by general formula (LXIV). Advantageously, the reaction can be carried out by adapting the protocols described by (Marx, Piantadosi et al. 1988) and (Gaffney and Reese 1997), in which PG-E can represent triphenylmethyl chloride or 9-phenylxanthene-9-ol or else 9-chloro-9-phenylxanthene;
d) deprotecting the sulfur atom of a compound (LXIV), in conditions known to those skilled in the art, to give a compound represented by general formula (LXV);
e) reacting a compound represented by general formula (LXV) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LXVI) in which R1 and R3, which are the same or different, represent a CO—R5 or CO—(CH₂)₂ₙ₊₁—X—R6 group;
f) deprotecting a compound represented by formula (LXVI), in conventional conditions known to those skilled in the art, to give a compound represented by general formula (IB) in which G2 is an oxygen atom, G3 is a sulfur atom, R and R2 are hydrogen atoms and R1 and R3, which are the same or different, represent a CO—R5 or CO—(CH₂)₂ₙ₊₁—X—R6 group;
g) reacting a compound represented by general formula (IB) in which G2 is an oxygen atom, G3 is a sulfur atom, R and R2 are hydrogen atoms and R1 and R3, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art. 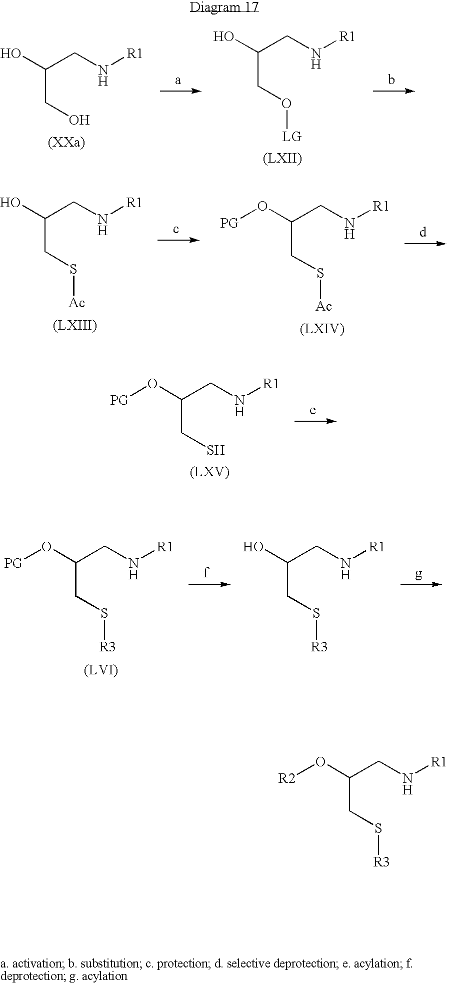
   Compounds represented by formula (IB) according to the invention (i) G2 is an oxygen atom, (ii) G3 is a sulfur atom, (iii) R and R3 are hydrogen atoms, (iv) R2 is a hydrogen atom or represents a CO—R5 or CO—(CH₂)₂ₙ₊₁—X—R6 group and (v) R1 represents a CO—R5 or CO—(CH₂)₂ₙ₊₁—X—R6 group, can be prepared from compounds having formula (LXV) by deprotecting the oxygen according to conventional conditions known to those skilled in the art.

Compounds represented by formula (IB) according to the invention in which (i) G2 is an oxygen atom, (ii) G3 is a sulfur atom, (iii) R is a hydrogen atom, (iv) R1 and R2 are hydrogen atoms or represent a CO—R5 or CO—(CH₂)₂ₙ₊₁—X—R6 group, which are the same or different, and (v) R3 represents a CO—R5 or CO—(CH₂)₂ₙ₊₁—X—R6 group, can be prepared from compounds having formula (XXIa) according to the following method (diagram 18):
a) reacting a compound represented by formula (XXIa) such as defined hereinabove, with a compound corresponding to the formula LG-E (in stoichiometric amounts) in which E represents a halogen and LG is a reactive group selected for example in the group consisting of mesyl, tosyl, etc., to give a compound represented by general formula (LXVII);
b) reacting a compound represented by formula (LXVII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (LXVIII). Advantageously, said reaction can be carried out by adapting the protocol described by (Gronowitz, Herslöf et al. 1978);
c) reacting a compound represented by formula (LXVIII) with a compound PG′-E in which PG′ is a protective group and E is a reactive group selected for example in the group consisting of OH and a halogen, to give a compound represented by general formula (LXIX). Advantageously, the reaction can be carried out by adapting the protocols described by (Marx, Piantadosi et al. 1988) and (Gaffney and Reese 1997) in which PG′-E can represent triphenylmethyl chloride or 9-phenylxanthene-9-ol or else 9-chloro-9-phenylxanthene;
d) deprotecting the sulfur atom of a compound (LXIX), in conditions known to those skilled in the art, to give a compound represented by general formula (LXX);
e) reacting a compound represented by general formula (LXX) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LXXI) in which R3 represents a CO—R5 or CO—(CH₂)₂ₙ₊₁—X—R6 group;
f) deprotecting a compound represented by formula (LXXI), in conventional conditions known to those skilled in the art, to give a compound represented by general formula (IB) in which G2 is an oxygen atom, G3 is a sulfur atom, R and R2 are hydrogen atoms and R3 represents a CO—R5 or CO—(CH₂)₂ₙ₊₁—X—R6 group (compound LXXII);
g) reacting a compound represented by formula (LXXII) with a compound (PG)₂O in which PG is a protective group to give a compound represented by general formula (LXXIII). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
h) reacting a compound represented by general formula (LXXIII) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (LXXIV);
i) deprotecting a compound (LXXIV) in conventional conditions known to those skilled in the art to give a compound represented by general formula (IB) in which G3 is a sulfur atom, G2 is an oxygen atom, R and R1 are hydrogen atoms and R2 and R3, which are the same or different, represent a CO—R5 or CO—(CH₂)₂ₙ₊₁—X—R6 group;
j) reacting a compound represented by general formula (IB) in which G3 is a sulfur atom, G2 is an oxygen atom, R and R1 are hydrogen atoms and R2 and R3, which are the same or different, represent a CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

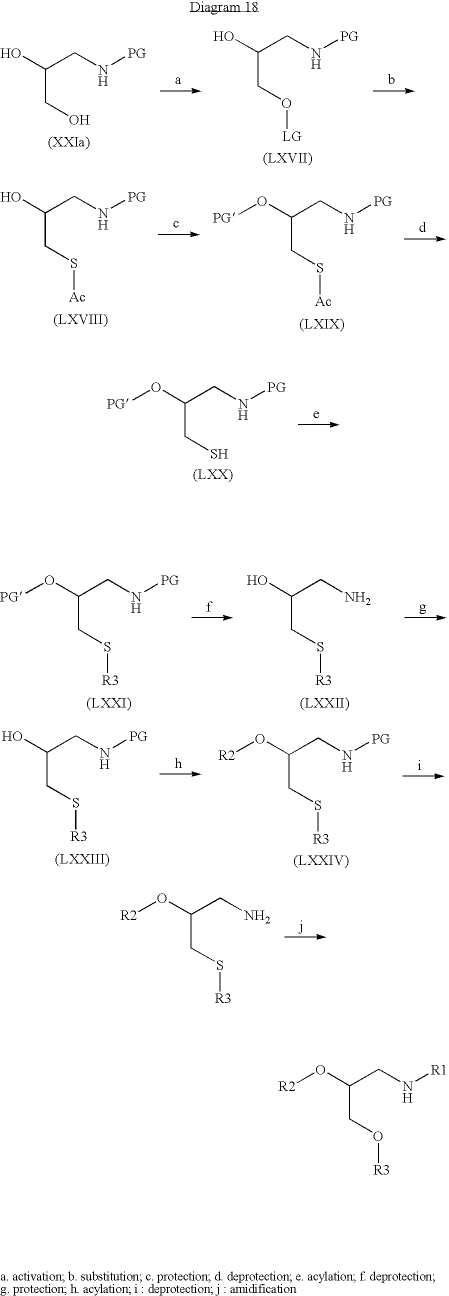

Compounds represented by formula (IB) according to the invention in which (i) G2 is an oxygen atom, (ii) G3 is a sulfur atom, (iii) R is a hydrogen atom, (iv) R2 and R3, which are the same, are hydrogen atoms or represent a CO—R5 or CO—(CH₂)₂ₙ₊₁—X—R6 group and (v) R1 represents a CO—R5 or CO—(CH₂)₂ₙ₊₁—X—R6 group, can be prepared from compounds having formula (IIIa) according to the following method (diagram 19):
a) reacting a compound represented by formula (XXIa) such as defined hereinabove, with a compound corresponding to the formula (LG)2 (in stoichiometric amounts) in which LG is a reactive group selected for example in the group consisting of iodine, bromine, etc., to give a compound represented by general formula (LXVIIa);
b) reacting a compound represented by formula (LXVIIa) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (LXVIII);
c) deprotecting the sulfur atom of a compound (LXVIII), in conditions known to those skilled in the art, to give a compound represented by general formula (LXXV);
d) reacting a compound represented by general formula (LXXV) with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LXXIV) in which R2 and R3 represent a same CO—R5 or CO—(CH₂)₂ₙ₊₁—X—R6 group;
e) deprotecting a compound represented by formula (LXXIV), in conventional conditions known to those skilled in the art, to give a compound represented by general formula (IB) in which G2 is an oxygen atom, G3 is a sulfur atom, R and R2 are hydrogen atoms and R2 and R3 represent a same CO—R5 or CO—(CH₂)₂ₙ₊₁—X—R6 group;
f) reacting a compound represented by general formula (IB) in which G2 is an oxygen atom, G3 is a sulfur atom, R and R2 are hydrogen atoms and R2 and R3 represent a same CO—R5 or CO—(CH₂)₂ₙ₊₁—X—R6 group with a compound corresponding to the formula A°-CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

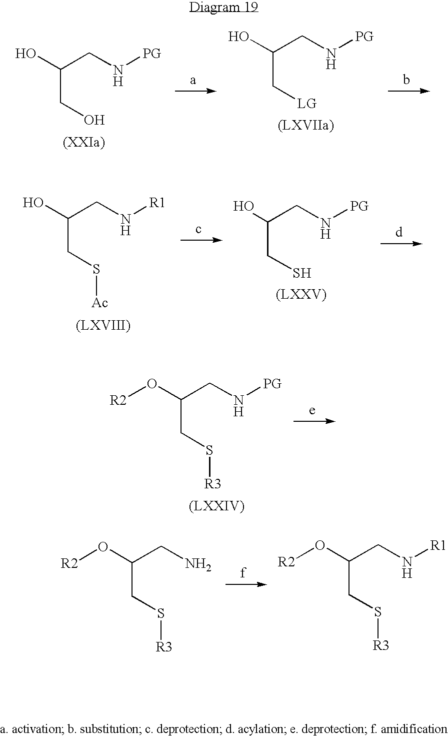

The feasibility, realization and other advantages of the invention are further detailed in the following examples, which are given for purposes of illustration and not by way of limitation.

### LEGENDS OF FIGURES

FIG. 1: Structure of inventive compounds.

FIG. 1A: Structure of acyl glycerols according to the invention whose preparation is described in examples 2a and 2c and 4a to 4r and respectively noted on the FIGS. as 1A.2a, 1A.2c, 1A.4a to 1A.4r.

FIG. 1B: Structure of particular inventive compounds whose preparation is described in examples 5a, 5b, 6c, 7, 9, 10, 11, 13, 15, 16 18, 19, 21, 23, 24, 26 and 28 and respectively noted on the FIGS. as 1B.5a, 1B.5b, 1B.6c, 1B.7, 1B.9, 1B.10, 1B.11, 1B.13, 1B.15, 1B.16, 1B.18, 1B.19, 1B.21, 1B.23, 1B.24, 1B.26 and 1B.28.

FIG. 2: Evaluation of the activity of the inventive compounds according to the formulation used (carboxymethylcellulose (CMC), Cremophor® RH40 and Solutol® HS15)

FIG. 2A: assay of total plasma cholesterol.

FIG. 2B : assay of plasma triglycerides.

FIG. 3: Evaluation of the PPARα agonist properties of the inventive compounds with the Gal4/PPARα transactivation system

FIG. 4: Evaluation of the antioxidant properties of the inventive compounds on LDL oxidation by copper (Cu).

FIG. 4a: conjugated diene formation over time or lag phase.

FIG. 4b: LDL oxidation rate.

FIG. 4c: maximum amount of conjugated dienes formed.

FIG. 5: Evaluation of the neuroprotective properties of inventive compound Ex 4a in a Parkinson's disease model.

FIG. 5A: number of apomorphine-induced rotations.

FIG. 5B: number of neurons immunohistochemically labelled with anti-tyrosine hydroxylase.

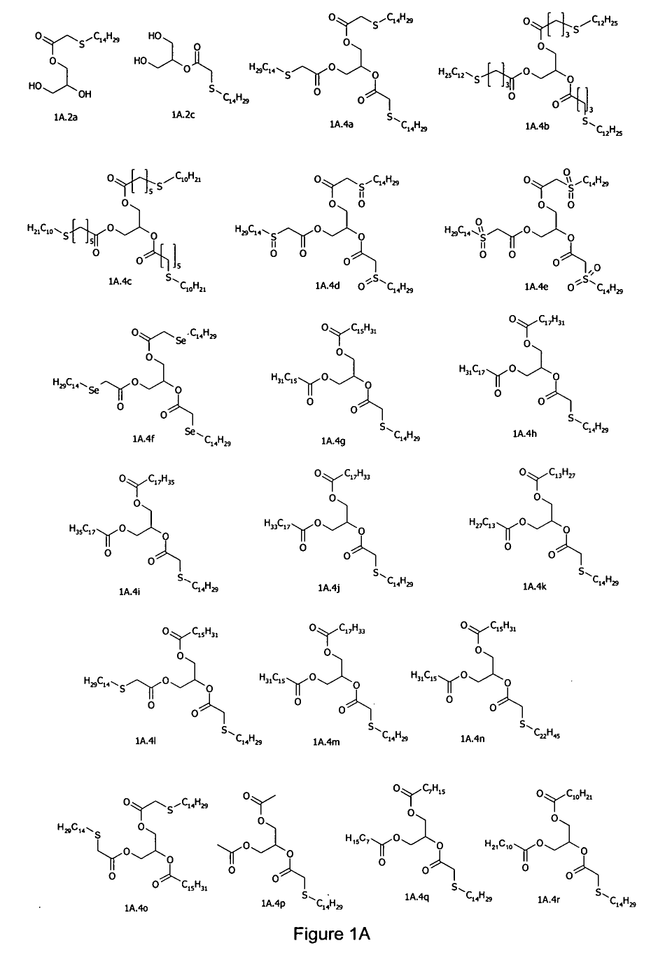

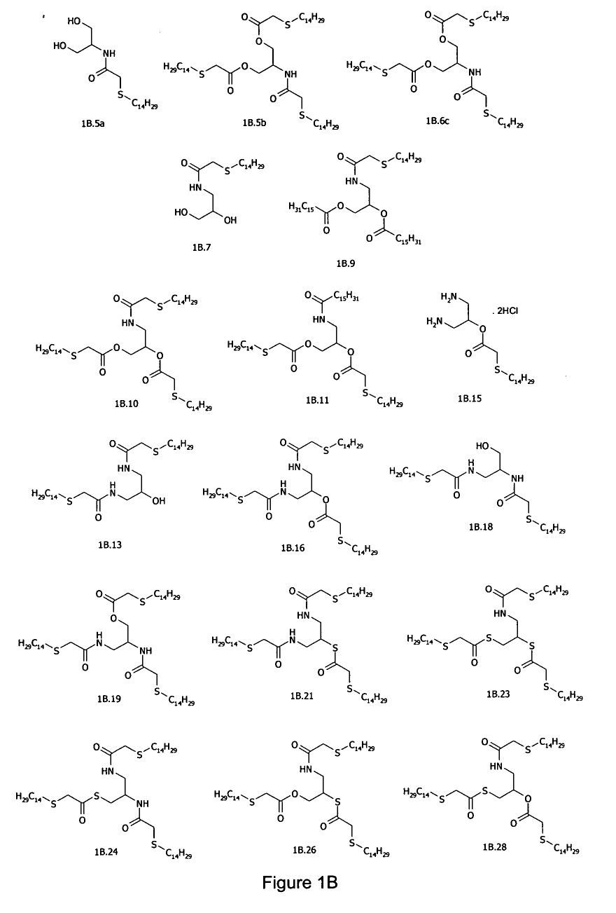

### EXAMPLES

For easier comprehension of the text, the inventive compounds used in the examples concerning the measurement and evaluation of activity are abbreviated as follows: “Ex 2”, for instance, indicates the inventive compound whose preparation is described by example 2.

Thin-layer chromatography (TLC) was carried out on plates coated with Merck silica gel 60F₂₅₄ 0.2 mm thick. Retention factor is abbreviated Rf.

Column chromatography was carried out on silica gel 60 with a particle size of 40-63 μm (Merck reference 9385-5000).

Melting points (MP) were determined on a Buchi B 540 apparatus by the capillary method.

Infrared (IR) spectra were recorded on a Bruker Fourier transformation spectrometer (Vector 22).

Nuclear magnetic resonance (NMR) spectra were recorded on a Bruker AC 300 spectrometer (300 MHz). Each signal was identified by its chemical shift, intensity, multiplicity (noted s for singlet, sl for broad singlet, d for doublet, dd for split doublet, t for triplet, td for split triplet, quint for quintuplet and m for multiplet) and its coupling constant (J).

Mass spectra (MS) were determined on a Perkin Elmer Sciex API 1 (ESI-MS for ElectroSpray Ionization Mass Spectrometry) or on an Applied Biosystems Voyager DE-STR of the MALDI-TOF type (Matrix-Assisted Laser Desorption/Ionization—Time Of Flight).

### Example 1

### Preparation of Fatty Acid Derivatives

### Example 1a: Preparation of Tetradecylthioacetic Acid

Potassium hydroxide (34.30 g, 0.611 mol), mercaptoacetic acid (20.9 ml, 0.294 mol) and 1-bromotetradecane (50 ml, 0.184 mol) were added in that order to methanol (400 ml). The mixture was stirred overnight at room temperature. A concentrated hydrochloric acid solution (60 ml) dissolved in water (800 ml) was then added. The tetradecylthioacetic acid precipitated. The mixture was stirred overnight at room temperature. The precipitate was then filtered, washed five times with water and dried in a dessicator. The product was recrystallized in methanol.

Yield: 94%

Rf (dichloromethane/methanol 9:1): 0.60

MP: 67-68° C.

IR: vCO acid 1726 and 1684 cm⁻¹

NMR (¹H, CDCl₃) : 0.84-0.95 (t, 3H, —CH₃, J=6.5 Hz); 1.20-1.45 (multiplet, 22H, —CH₂—); 1.55-1.69 (quint, 2H, —CH₂—CH₂—S—, J=7 Hz); 2.63-2.72 (t, 2H, CH₂—CH₂—S—, J=7 Hz); 3.27 (s, 2H, S—CH₂—COOH).

MS (ESI-MS): M−1=287

### Example 1b

### Preparation of 4-(dodecylthio)butanoic Acid

Dodecanethiol (2.01 g, 10 mmol) and ethyl bromobutyrate (1.971 g, 10 mmol) were stirred at room temperature in an inert atmosphere. Potassium hydroxide (1.36 g, 21 mmol) dissolved in 50 ml of ethanol was added slowly. The reaction mixture was refluxed for 3 hours and the ethanol was vacuum evaporated. The residue was taken up in water and acidified. The precipitate which formed was filtered, washed with water and dried.

Yield: 90%

Rf (dichloromethane/methanol 9:1): 0.46

IR: vCO acid 1689 cm⁻¹

NMR (¹H, CDCl₃) : 0.86-0.91 (t, 3H, —CH₃, J=6.2 Hz); 1.25-1.45 (multiplet, 18H, —CH₂—); 1.53-1.63 (quint, 2H, —CH₂—CH₂—S—, J=6.7 Hz); 1.87-2.00 (quint, 2H, —CH₂—S—CH₂—CH₂—CH₂—COOH, J=7.2 Hz); 2.47-2.55 (m, 4H, —CH₂—S—CH₂—CH₂—CH₂—COOH); 2.55-2.62 (t, 2H, —CH₂—S—CH₂—CH₂—CH₂—COOH, J=7.2 Hz).

MS (ESI-MS): M−1=287

### Example 1c

### Preparation of 6-(decylthio)hexanoic Acid

Decanethiol (4.57 g, 25 mmol) and 4-bromobutyric acid (5 g, 25 mmol) were stirred at room temperature in an inert atmosphere. Potassium hydroxide dissolved in 50 ml of ethanol was added slowly. The reaction mixture was refluxed for 3 hours and the ethanol was vacuum evaporated. The residue was taken up in water and acidified. The precipitate which formed was filtered, washed with water and dried.

Yield: 95%

Rf (dichloromethane/methanol 9:1): 0.37

IR: vCO acid 1690 cm⁻¹

NMR (¹H, CDCl₃): 0.86-0.91 (t, 3H, —CH₃, J=6.5 Hz); 1.22-1.41 (multiplet, 14H —CH₂—); 1.42-1.50 (m, 2H, CH₂—S—CH₂—CH₂—CH₂—CH₂—CH₂—COOH); 1.53-1.75 (multiplet, 6H, —CH₂—CH₂—S—CH₂—CH₂—CH₂—CH₂—CH₂—COOH); 2.35-2.42 (t, 2H —CH₂—S—CH₂—CH₂—CH₂—CH₂—CH₂—COOH, J=7 Hz); 2.48-2.55 (multiplet, 4H, —CH₂—S—CH₂—).

MS (ESI-MS): M−1=287

### Example 1d

### Preparation of Tetradecylselenoacetic Acid

Preparation of tetradecyldiselenide Under an inert atmosphere, selenium (1.19 g, 15 mmol) was added to a 1:1 mixture of tetrahydrofuran/water (50 ml). The reaction mixture was cooled in an ice bath before slowly adding sodium tetraborohydride (1.325 g, 35 mmol). A second fraction of selenium (1.19 g, 15 mmol) was added. The reaction mixture was stirred at room temperature for 15 min then heated under reflux to dissolve all the reagents. Bromotetradecane (9 ml, 30 mmol) dissolved in 25 ml of tetrahydrofuran was added. The reaction mixture was stirred at room temperature for 3 hours, then extracted with dichloromethane. The organic phases were combined, dried on magnesium sulfate, filtered and dried. The product was used without further purification.

Rf (petroleum ether): 0.77

MP: 43° C.

IR: vCH 2960-2850 cm⁻¹

NMR (¹H, CDCl₃): 0.87-0.93 (t, 6H, —CH₃, J=6.5 Hz); 1.20-1.48 (multiplet, 44H, —CH₂—); 1.62-1.80 (m, 4H, —CH₂—CH₂—Se—); 2.88-2.96 (t, 4H, —CH₂—CH₂—Se—, J=7 Hz).

### Preparation of Tetradecylselenoacetic Acid

Under an inert atmosphere, ditetradecyldiselenide (8.5 g, 17 mmol) was dissolved in a mixture of tetrahydrofuran/water (150 ml/50 ml) and cooled in an ice bath. Sodium tetraborohydride (2.9 g, 61 mmol) was added slowly (the solution blanched) followed by the addition of bromoacetic acid (8.5 g, 61 mmol) dissolved in a mixture of tetrahydrofuran/water (25 ml/25 ml). The reaction mixture was stirred at room temperature for 6 hours, then extracted with ether. The aqueous phase was acidified. The resulting precipitate was filtered, washed several times with water and dried.

Yield: 29%

Rf (dichloromethane/methanol 9:1): 0.60

MP: 68° C.

IR: vCO acid 1719 and 1680 cm⁻¹

NMR (¹H, CDCl₃): 0.85-0.95 (t, 3H, —CH₃, J=6.5 Hz); 1.25-1.48 (multiplet, 22H —CH₂—); 1.65-1.78 (quint, 2H, —CH₂—CH₂—Se—, J=6.5 Hz); 2.78-2.84 (t, 2H, CH₂—CH₂—Se—, J=7 Hz); 3.18 (s, 2H, Se—CH₂—COOH).

MS (ESI-MS): M−1=335

### Example 1e

### Preparation of Tetradecylsulfoxyacetic Acid

Tetradecylthioacetic acid (example 1a) (5 g, 17.4 mmol) was dissolved in a mixture of methanol/dichloromethane (160 ml/80 ml). The reaction mixture was cooled in an ice bath with stirring followed by the slow addition of Oxone® (12.8 g, 21 mmol) dissolved in water (160 ml). The reaction mixture was stirred at room temperature for 3 hours. The solvents were vacuum evaporated. The precipitate which formed in the residual aqueous phase was drained, washed several times with water and dried.

Yield: 90%

Rf (dichloromethane/methanol 9:1): 0.27

IR: vCO acid 1723 and 1690 cm⁻¹

NMR (¹H, DMSO): 0.80-0.92 (t, 3H, —CH₃, J=6.4 Hz); 1.19-1.50 (multiplet, 22H, —CH₂—); 1.55-1.71 (quint, 2H, —CH₂—CH₂—SO—); 2.70-2.89 (t, 2H, —CH₂—CH₂—SO—CH₂—COOH, J=6.7Hz); 3.52-3.70 (d, 1H, —CH₂—SO—CH₂—COOH, J=14.5 Hz); 3.80-3.95 (d, 1H, —CH₂—SO—CH₂—COOH, J=14.1 Hz).

MS (ESI-MS): M+1=305; M+23=327 (M+Na⁺); M+39=343 (M+K⁺)

### Example 1f

### Preparation of 6-(decylsulfoxy)hexanoic Acid

This compound was synthesized according to the method described hereinabove (example 1e) from 6-(decylthio)hexanoic acid (example 1c).

Yield: 94%

Rf (dichloromethane/methanol 9:1): 0.18

NMR (¹H, CDCl₃): 0.86-0.91 (t, 3H, —CH₃, J=6.8 Hz); 1.20-1.40 (multiplet, 14H, —CH₂—); 1.40-1.60 (m, 2H, CH₂—SO—CH₂—CH₂—CH₂—CH₂—CH₂-COOH); 1.63-1.95 (multiplet, 6H, —CH₂—CH₂—SO—CH₂—CH₂—CH₂—CH₂—CH₂—COOH); 2.35-2.42 (m, 3H, —CH₂—SO—CH₂—CH₂—CH₂—CH₂—CH₂—COOH and —CH₂—SO—CH₂—CH₂—CH₂—CH₂—CH₂—COOH); 2.60-2.71 (m, 1H, —CH₂—SO—CH₂—CH₂—CH₂—CH₂—CH₂—COOH); 2.75-2.85 (m, 1 H, —CH₂—SO—(CH₂)₅—COOH); 2.80-3.01 (m, 1 H, —CH₂—SO—(CH₂)₅—COOH).

### Example 1g

### Preparation of tetradecylsulfonylacetic Acid

Tetradecylthioacetic acid (example 1a) (5 g, 17.4 mmol) was dissolved in a mixture of methanol/dichloromethane (160 ml/80 ml). The reaction mixture was cooled in an ice bath with stirring followed by the slow addition of Oxone® (21.8 g, 35 mmol) dissolved in water (160 ml). The reaction mixture was stirred at room temperature for 3 hours. The solvents were vacuum evaporated. The precipitate which formed in the residual aqueous phase was drained, washed several times with water and dried

Yield: 89%

Rf (dichloromethane/methanol 9:1): 0.21

IR: vCO acid 1701 cm⁻¹

NMR (¹H, DMSO): 0.85-0.96 (t, 3H, —CH₃, J=6 Hz); 1.20-1.40 (multiplet, 20H, —CH₂—); 1.40-1.55 (m, 2H, —CH₂—CH₂—CH₂—SO₂—); 1.80-1.96 (m, 2H, —CH₂—CH₂—SO₂—); 3.22-3.34 (t, 2H, —CH₂—CH₂—SO₂—CH₂—COOH, J=8 Hz); 4.01 (s, 2H, —CH₂—SO₂—CH₂—COOH).

MS (ESI-MS): M−1=319

### Example 1h

### Preparation of 6-(decylsulfonyl)hexanoic Acid

This compound was synthesized according to the method described hereinabove (example 1g) from 6-(decylthio)hexanoic acid (example 1c).

Yield: 87%

Rf (dichloromethane/methanol 9:1): 0.15

IR: vCO acid 1689 cm⁻¹

NMR (¹H, CDCl₃): 0.85-0.96 (t, 3H, —CH₃, J=6.5 Hz); 1.22-1.40 (multiplet, 14H, —CH₂—); 1.40-1.61 (m, 2H, —SO₂—CH₂—CH₂—CH₂—); 1.65-1.95 (multiplet, 6H, —CH₂—CH₂—SO₂—CH₂—CH₂—CH₂—CH₂—CH₂—COOH); 2.35-2.46 (m, 2H, —CH₂—COOH); 2.60-2.84 (m, 2H, —CH₂—SO₂—CH₂—CH₂—CH₂—CH₂—CH₂—COOH); 2.90-3.02 (m, 2H, —CH₂—SO₂—CH₂—CH₂—CH₂—CH₂—CH₂—COOH).

### Example 11

### Preparation of Docosylthioacetic Acid

This compound was synthesized according to the method described hereinabove (example 1a) from mercaptoacetic acid and bromodocosane.

Yield: 90%

Rf (dichloromethane/methanol 9:1): 0.62

IR: vCO acid 1728 and 1685 cm⁻¹

NMR (¹H, CDCl₃): 0.83-0.94 (t, 3H, —CH₃, J=6.6 Hz); 1.18-1.48 (multiplet, 38H, —CH₂—); 1.55-1.69 (quint, 2H, —CH₂—CH₂—S—, J=7 Hz); 2.63-2.72 (t, 2H, CH₂—CH₂—S—, J=7 Hz); 3.26 (s, 2H, S—CH₂—COOH).

### Example 2

### Preparation of Monoacylglycerols

### Example 2a

### Preparation of 1-tetradecylthioacetylglycerol

### Preparation of 1-tetradecylthioacetate-2.3-isopropylideneglycerol

In a flask immersed in an ice bath, tetradecylthioacetic acid (example 1a) (4 g, 13.86 mmol) was dissolved in tetrahydrofuran (100 ml) followed by the addition of EDCl (1-(3-dimethylaminopropyl)-3-ethylcarbodiimide hydrochloride) (2.658 g, 13.86 mmol), dimethylaminopyridine (1.694 g, 13.86 mmol) and solketal (1.72 ml, 13.86 mmol) in that order. The reaction mixture was stirred at room temperature for 4 days. The solvent was vacuum evaporated. The residue was taken up in dichloromethane, washed with aqueous 1N hydrochloric acid solution then with 10% sodium bicarbonate and lastly with a saturated sodium chloride solution. The organic phase was dried on magnesium sulfate, filtered and vacuum evaporated. The oily residue obtained was purified by chromatography on silica gel (ethyl acetate/cyclohexane 1:9). The product was obtained in the form of a yellow oil.

Yield: 80%

Rf (cyclohexane/ethyl acetate 8:2): 0.65

IR: vCO ester 1736 cm⁻¹

NMR (¹H, CDCl₃): 0.86 (t, 3H, —CH₃, J=7.8 Hz); 1.25 (multiplet, 20H, —CH₂—); 1.33 (s, 3H, CH₃ isopropylidene); 1.37 (s, 3H, CH₃ isopropylidene); 1.59 (m, 4H, OCO—CH₂—S—CH₂—CH₂—CH₂—); 2.62 (t, 2H, —O—CO—CH₂—S—CH₂—, J=7.4 Hz); 3.25 (s, 2H, —O—CO—CH₂—S—CH₂—); 3.75 (m, 1H, —CO—O—CH₂—CH(O)—CH₂(O) (isopropylidene)); 4.08 (m, 2H, —CO—O—CH₂—CH(O)—CH₂(O)— (isopropylidene)); 4.18 (m, 1H, —CO—O—CH₂—CH(O)—CH₂(O)— (isopropylidene); 4.35 (m, 1H, —CO—O—CH₂—CH(O)—CH₂(O)— (isopropylidene)).

### Preparation of 1-tetradecylthioacetylglycerol

1-tetradecylthioacetyl-2,3-isopropylideneglycerol (4.163 g, 10.356 mmol) was dissolved in acetic acid (60 ml) and stirred at room temperature. After 1 week of reaction, the mixture was diluted with water and extracted with ethyl acetate. The organic phase was washed with saturated sodium chloride solution then dried on magnesium sulfate, filtered and the solvent evaporated. The resulting white powder was recrystallized in heptane.

Yield: 90%

Rf (ethyl acetate/cyclohexane 5:5): 0.30

MP: 63-65° C.

IR: vCO ester 1720 cm⁻¹

NMR (¹H, CDCl₃): 0.89 (t, 3H, —CH₃, J=6.6 Hz); 1.28 (multiplet, 20H, —CH₂—); 1.59 (multiplet, 4H, —CH₂—CH₂—CH₂—S—); 2.64 (t, 2H, CH₂—CH₂—S—, J=7. 2Hz); 3.26 (s, 2H, S—CH₂—COO); 3.64 (m, 2H, —COO—CH₂—CHOH—CH₂OH); 3.97 (m, 1H, —COO—CH₂—CHOH—CH₂OH); 4.27 (m, 2H, —COO—CH₂—CHOH—CH₂OH).

MS (MALDI-TOF): M+23=385 (M+Na⁺)

### Example 2b

### Preparation of 1-palmitoylglycerol

This compound was synthesized according to the method described hereinabove (example 2a) from solketal and palmitic acid.

### Preparation of 1-palmitoyl-(2.3-isopropylidene)-glycerol

Yield: 55%

Rf (dichloromethane): 0.35

MP: 32-33° C.

IR: vCO ester 1733 cm⁻¹

NMR (¹H, CDCl₃): 0.89 (t, 3H, —CH₃, J=6.6 Hz); 1.27 (multiplet, 24H, —CH₂—); 1.39 (s, 3H, CH₃ isopropylidene); 1.45 (s, 3H, CH₃ isopropylidene); 1.62 (m, 2H, OCO—CH₂—CH₂—CH₂—); 2.32 (t, 2H, —O—CO—CH₂—CH₂—CH₂—, J=7.4 Hz); 3.75 (dd, 1H, CO—O—CH₂—CH(O)—CH₂(O) (isopropylidene), J=8.3 Hz and J=2.1 Hz); 4.10 (m, 2H, —CO—O—CH₂—CH(O)—CH₂(O)— (isopropylidene)); 4.18 (dd, 1H, —CO—O—CH₂—CH(O)—CH₂(O)— (isopropylidene), J=11.6 Hz and J=4.6 Hz); 4.33 (m, 1 H, —CO—O—CH₂—CH(O)—CH₂(O)— (isopropylidene)).

### Preparation of 1-palmitoylglycerol

Yield: 84%

Rf (ethyl acetate/cyclohexane 5:5): 0.30

MP: 72-74° C.

IR: vCO ester 1730 cm⁻¹

NMR (¹H, CDCl₃): 0.89 (t, 3H, —CH₃, J=6.5 Hz); 1.26 (multiplet, 24H, —CH₂—); 1.64 (m, 2H, OCO—CH₂—CH₂—CH₂—); 2.36 (t, 2H, —O—CO—CH₂—CH₂—CH₂—, J=7.4 Hz); 3.60 (dd, 1 H, —CO—O—CH₂—CHOH—CH₂OH, J=11.8 Hz and J=6.1 Hz); 3.71 (dd, 1H, —CO—O—CH₂—CHOH—CH₂OH, J=11.8 Hz and J=3.9 Hz); 3.94 (m, 1H, —CO—O—CH₂—CHOH—CH₂OH); 4.19 (m, 2H, —CO—O—CH₂—CHOH—CH₂OH).

### Example 2c

### Preparation of 2-tetradecylthioacetylglycerol

### Preparation of 1.3-benzylideneglycerol

Glycerol (30 g, 0.326 mol), benzaldehyde (34.5 g, 0.326 mol) and p-toluene sulfonic acid (50 mg) were dissolved in 350 ml of toluene and refluxed in a Dean-Stark apparatus for 18 hours. The reaction mixture was dried and the residual product purified by chromatography on silica gel (eluent:cyclohexane/ethyl acetate 8:2 then 7:3) then recrystallized.

Yield: 20%

Rf (ethyl acetate/cyclohexane 5:5): 0.34

IR: vOH 3286 cm⁻¹

NMR (¹H, CDCl₃): 3.19 (sl, 1H exchangeable, —OH); 3.64 (sl, 1 H, —O—CH₂—CHOH—CH₂O—); 3.99-4.16 (dd, 2H, —O—CHaHb-CHOH—CHaHbO—, J=1.1 Hz and J=10.4 Hz); 4.17-4.23 (dd, 2H, —O—CHaHb-CHOH—CHaHbO—, J=1.6 Hz and J=11.5 Hz); 5.57 (s, 1H, φ-CH—); 7.34-7.45 (m, 3H, aromatic H); 7.49-7.55 (m, 2H, aromatic H).

### Preparation of 2- tetradecylthioacetyl-1 3-benzylideneglycerol

In a flask immersed in an ice bath, tetradecylthioacetic acid (example 1a) (0.800 g, 2.774 mmol) was dissolved in tetrahydrofuran (75 ml) followed by the addition of EDCl (0.532 g, 2.774 mmol), dimethylaminopyridine (0.339 g, 2.774 mmol) and 1,3-benzylideneglycerol (0.5 g, 2.774 mmol) in that order. The mixture was stirred at room temperature for 16 hours. The solvent was evaporated. The resudue obtained was taken up in dichloromethane, washed with 1N hydrochloric acid then with 10% potassium carbonate and lastly with a saturated aqueous sodium chloride solution. The organic phase was dried on magnesium sulfate, filtered and dried. The residue was taken up in petroleum ether. The precipitate which formed was filtered and purified by chromatography on silica gel (eluent:ethyl acetate/cyclohexane 2:8) to give the desired product in the form of a white powder.

Yield: 50%

Rf (ethyl acetate/cyclohexane 2:8): 0.53

MP: 51-53° C.

IR: vCO ester 1723 cm⁻¹

NMR (¹H, CDCl₃): 0.85-0.96 (t, 3H, CH₃, J=6.8 Hz); 1.19-1.44 (multiplet, 20H, —CH₂); 1.52-1.69 (multiplet, 4H, —CH₂—CH₂—CH₂—S—); 2.62-2.80 (t, 2H, —CH₂—CH₂—CH₂—S—, J=7.2 Hz); 3.34 (s, 2H, —CH₂—S—CH₂—COO—); 4.12-4.29 (dd, 2H, —O—CHaHb-CH(OCO)—CHaHbO—, J=1.7 Hz and J=13.1 Hz); 4.30-4.41 (dd, 2H, —O—CHaHb-CH(OCO)—CHaHbO—, J=1.3 Hz and J=13.1 Hz); 4.75-4.79 (t, 1H, —O—CH₂—CH(OCO)—CH₂O—, J=1.7 Hz); 5.59 (s, 1H, φ-CH—); 7.35-7.45 (m, 3H, aromatic H); 7.48-7.57 (m, 2H, aromatic H).

### Preparation of 2-tetradecylthioacetylglycerol

2-tetradecylthioacetyl-1,3-benzylideneglycerol (0.576 g, 1.278 mmol) was dissolved in a 50:50 (VN) mixture of dioxane and triethylborate. Boric acid (0.317 g, 5.112 mmol) was added and the reaction mixture was heated at 100° C. for 4 hours. Two equivalents of boric acid (0.158 g, 2.556 mmol) were then added followed by 2 equivalents after 5.5 hours and 7 hours of reaction. After 24 hours of reaction, the triethylborate was evaporated. The residue was taken up in ethyl acetate and washed with water. The aqueous phase was neutralized with sodium bicarbonate then extracted with dichloromethane. The organic phase was washed with water saturated with sodium chloride, dried on magnesium sulfate, filtered and dried. The residue was purified by chromatography on silica gel (eluent:ethyl acetate/cyclohexane 5:5).

Yield: 62%

Rf (ethyl acetate/cyclohexane 7:3): 0.51

IR: vCO ester 1739 cm⁻¹

NMR (¹H, CDCl₃): 0.82-0.95 (t, 3H, —CH₃, J=6.9 Hz); 1.15-1.35 (multiplet, 22H, —CH₂—); 1.55-1.68 (m, 2H, —CH₂—CH₂—S—); 2.23 (sl, 2H, OH); 2.65 (m, 2H, CH₂—CH₂—S—); 3.26 (s, 2H, S—CH₂—COO); 3.64-3.73 (m, 4H, HOCH₂—CH(OCO—R)—CH₂OH); 3.97 (m, 1H, HOCH₂—CH(OCO—R)—CH₂OH).

### Example 3

### Preparation de 1,3-diacylalycerols

### Example 3a

### Preparation of 1.3-dipalmitoylglycerol

Glycerol (10 g, 0.109 mol, 1 eq), palmitic acid (55.69 g, 0.217 mol, 2 eq), dicyclohexylcarbodiimide (44.77 g, 0.217 mol, 2 eq) and dimethylaminopyridine (26.51 g, 0.217 mol, 2 eq) were dissolved in dichloromethane. The reaction mixture was stirred at room temperature for 48 hours. The dicyclohexylurea which formed was filtered and washed several times with dichloromethane. The filtrate was dried. The residual product was purified by silica gel chromatography (eluent:dichloromethane).

Yield: 45%

Rf (dichloromethane): 0.30

MP: 70-73° C.

IR: vCO ester 1735 and 1716 cm⁻¹

NMR (¹H, CDCl₃): 0.86-91 (t, 6H, —CH₃, J=6.5 Hz); 1.27 (multiplet, 48H, —CH₂—); 1.60-1.65 (quint, 4H, OCOCH₂—CH₂—, J=7.4 Hz); 2.32-2.38 (t, 4H, OCOCH₂—CH₂—, J=7.6 Hz); 2.51-2.52 (d, 1H, OH (exchangeable)); 4.06-4.21 (multiplet, 5H, —CH₂—CH—CH₂—).

MS (MALDI-TOF): M+23=591 (M+Na⁺); M+39=607 (M+K⁺)

### Example 3b

### Preparation of 1,3-dilinoleoylglycerol

This compound was obtained according to the method described hereinabove (example 3a) from glycerol and linoleic acid. The product was obtained as a colorless oil.

Yield: 26%

Rf (dichloromethane): 0.30

IR: vCO ester 1743 and 1719 cm⁻¹

NMR (¹H, CDCl₃): 0.83-0.93 (t, 6H, —CH₃, J=6.5 Hz); 1.15-1.44 (multiplet, 28H, —CH₂—); 1.55-1.70 (quint, 4H, OCOCH₂—CH₂—, J=7.4 Hz); 1.90-2.15 (multiplet, 8H, —CH₂—CH═CH—CH₂—CH═CH—CH₂—); 2.30-2.41 (t, 4H, OCOCH₂—CH₂—, J=7.6 Hz); 2.48-2.52 (d, 1H, OH (exchangeable)); 2.70-2.83 (t, 4H, —CH₂—CH═CH—CH₂—CH═CH—CH₂—); 4.05-4.25 (multiplet, 5H, —CHaHb-CH—CHaHb-); 5.25-5.46 (m, 8H, —CH₂—CH═CH—CH₂—CH═CH—CH₂—).

MS: M+23=639 (M+Na⁺); M+39=655 (M+K⁺)

### Example 3c

### Preparation of 1.3-distearoylglycerol

This compound was obtained according to the method described hereinabove (example 3a) from glycerol and stearic acid. The product was obtained as a white powder.

Yield: 21%

Rf (dichloromethane): 0.30

IR: vCO ester 1735 and 1716 cm⁻¹

NMR (¹H, CDCl₃): 0.83-0.91 (t, 6H, —CH₃, J=6.5 Hz); 1.27 (multiplet, 56H, —CH₂—); 1.59-1.66 (quint, 4H, OCOCH₂—CH₂—, J=7.4 Hz); 2.33-2.38 (t, 4H, OCOCH₂—CH₂—, J=7.5 Hz); 2.45-2.47 (d, 1H, OH (exchangeable), J=4.3 Hz); 4.08-4.23 (multiplet, 5H, —CHaHb-CH—CHaHb-).

MS (MALDI-TOF): M+23=647 (M+Na⁺)

### Example 3d

### Preparation of 1.3-dioleoylglycerol

This compound was obtained according to the method described hereinabove (example 3a) from glycerol and oleic acid. The product was obtained as a colorless oil.

Yield: 15%

Rf (dichloromethane): 0.23

IR: vCO ester 1743 and 1720 cm⁻¹

NMR (¹H, CDCl₃): 0.89 (t, 6H, —CH₃, J=7.2 Hz); 1.30 (multiplet, 40H, —CH₂—); 1.64 (quint, 4H, OCOCH₂—CH₂—, J=7.4 Hz); 2.02 (multiplet, 8H, —CH₂—CH═CH—CH₂—); 2.36 (t, 4H, OCOCH₂—CH₂—, J=7.2 Hz); 2.45 (d, 1 H, OH (exchangeable), J=4.2 Hz); 4.18 (multiplet, 5H, —CHaHb-CH—CHaHb-); 5.35 (m, 4H, —CH₂—CH═CH—CH₂—).

MS (MALDI-TOF): M+23=643 (M+Na⁺)

### Example 3e

### Preparation of 1.3-ditetradecanoylglycerol

This compound was obtained according to the method described hereinabove (example 3a) from glycerol and tetradecanoic acid. The product was obtained as a white powder.

Yield: 30%

Rf (dichloromethane): 0.30

IR: vCO ester 1733 and 1707 cm⁻¹

NMR (¹H, CDCl₃): 089 (t, 6H, —CH₃, J=6.5 Hz); 1.26 (multiplet, 40H, —CH₂—); 1.62 (quint, 4H, OCOCH₂—CH₂—, J=7.4 Hz); 2.36 (t, 4H, OCOCH₂—CH₂—, J=7.5 Hz); 2.45 (d, 1H, OH (exchangeable), J=4.3 Hz); 4.15 (multiplet, 5H, —CHaHb-CH—CHaHb-).

### Example 3f

### Preparation de 1.3-ditetradecylthioacetylglycerol

This compound was obtained according to the method described hereinabove (example 3a) from glycerol and tetradecylthioacetic acid (example 1a). The product was obtained as a white powder.

Yield: 37%

Rf (dichloromethane): 0.27

MP: 71-73° C.

IR: vCO ester 1704 cm⁻¹

NMR (¹H, CDCl₃): 089 (t, 6H, —CH₃, J=6.3 Hz); 1.27 (multiplet, 44H, —CH₂—); 1.58-1.63 (m, 4H, —OCO—CH₂—S—CH₂—CH₂—); 2.64 (t, 4H, —OCO—CH₂—S—CH₂—CH₂—, J=7.4 Hz); 3.26 (s, 4H, —OCO—CH₂—S—CH₂—); 4.16-4.29 (multiplet, 5H, —CHaHb-CH—CHaHb-).

### Example 3g

### Preparation of 1-oleoyl-3-palmitoylglycerol

Glycerol 1-palmitate (example 2b) (5.516 g, 17 mmol) was dissolved in dichloromethane (500 ml). Dicyclohexylcarbodiimide (5.165 g, 25 mmol), dimethylaminopyridine (3.058 g, 25 mmol) and oleic acid (4.714 g, 17 mmol) were then added. The reaction mixture was stirred at room temperature for 24 hours. The dicyclohexylurea precipitate was filtered, washed with dichloromethane and the filtrate was vacuum evaporated. The residue obtained was purified by silica gel chromatography (eluent:dichloromethane) to give the desired compound as a white solid.

Yield: 23%

Rf (dichloromethane): 0.24

MP: 30° C.

IR: vCO ester 1731 and 1710 cm⁻¹

NMR (¹H, CDCl₃): 087 (t, 6H, —CH₃, J=6.5 Hz); 1.26 (multiplet, 44H, —CH₂—); 1.62 (quint, 4H, OCOCH₂—CH₂—, J=7.4 Hz); 2.01 (multiplet, 4H, —CH₂—CH═CH—CH₂—); 2.36 (t, 4H, OCOCH₂—CH₂—, J=7.3 Hz); 2.465 (d, 1H, OH (exchangeable), J=4.3Hz); 4.17 (multiplet, 5H, —CHaHb-CH—CHaHb-); 5.34 (m, 4H, —CH₂—CH═CH—CH₂—).

MS (MALDI-TOF): M+23=617 (M+Na⁺)

### Example 3h

### Preparation of 1.3-diacetylglycerol

Glycerol (30 g, 0.326 mol) was dissolved in dichloromethane (300 ml) followed by addition of pyridine (79 ml, 0.977 mol) and then dropwise addition of acetic anhydride (61.5 ml, 0.651 mol). The reaction mixture was stirred at room temperature for 48 hours. The mixture was taken up in dichloromethane. The organic phase was washed with 1N hydrochloric acid followed by 10 % sodium bicarbonate and lastly with a saturated aqueous sodium chloride solution, dried on magnesium sulfate, filtered, and evaporated to dryness to provide a colorless oil which was used without further purification.

Yield: 34%

IR: vCO ester 1742 cm⁻¹

### Example 3i

### Preparation of 1.3-dioctanoylglycerol

This compound was obtained according to the method described hereinabove (example 3a) from glycerol and octanoic acid. The product was obtained as a colorless oil.

Yield: 10%

Rf (ethyl acetate/cyclohexane 3:7): 0.55

MP<4° C.

IR: vCO ester 1742 and 1719 cm⁻¹

NMR (¹H, CDCl₃): 0.89 (t, 6H, —CH₃, J=6.9 Hz); 1.29 (multiplet, 16H, —CH₂—); 1.62 (multiplet, 4H, OCOCH₂—CH₂—); 2.36 (t, 4H, OCOCH₂—CH₂—, J=7.4 Hz); 2.52 (sl, 1H, OH (exchangeable)); 4.14 (multiplet, 5H, —CH₂—CH—CH₂—).

MS (MALDI-TOF): M+23=591 (M+Na⁺); M+39=607 (M+K⁺)

### Example 3j

### : Preparation of 1.3-diundecanoylglycerol

This compound was obtained according to the method described hereinabove (example 3a) from glycerol and undecanoic acid. The product was obtained as a white powder.

Yield: 28%

Rf (dichloromethane): 0.20

IR: vCO ester 1730 and 1705 cm⁻¹

NMR (¹H, CDCl₃): 0.89 (t, 6H, —CH₃, J=6.7 Hz); 1.27 (multiplet, 28H, —CH₂—); 1.64 (m, 4H, OCOCH₂—CH₂—); 2.36 (t, 4H, OCOCH₂—CH₂—, J=7.4Hz); 4.18 (multiplet, 5H, —CH₂—CH—CH₂—).

MS (MALDI-TOF): M+23=451 (M+Na⁺); M+39=467 (M+K⁺)

### Example 4

### Preparation of 1,2,3-triacylglycerols

### Example 4a

### Preparation of 1,2,3-tritetradecylthioacetylglycerol

Glycerol (1 g, 10.86 mmol) was dissolved in dichloromethane (200 ml). Dicyclohexylcarbodiimide (7.84 g, 38.01 mmol), dimethylaminopyridine (4.64 g, 38.01 mmol) and tetradecylthioacetic acid (example 1a) (9.40 g, 32.58 mmol) were then added. The mixture was stirred at room temperature. After 48 hours of reaction, the dicyclohexylurea precipitate was filtered, washed with dichloromethane and the filtrate was evaporated. The residue obtained was purified by silica gel chromatography (eluent: dichloromethane/cyclohexane 4:6). 1,2,3-tritetradecylthioacetylglycerol was obtained as a white powder.

Yield: 65%

Rf (dichloromethane/cyclohexane 7:3): 0.47

MP: 57° C.

IR: vCO ester 1738 and 1722 cm⁻¹

NMR (¹H, CDCl₃): 0.89 (t, 9H, —CH₃, J=6.5 Hz); 1.26 (multiplet, 66H, —CH₂—); 1.62 (m, 6H, —CH₂—CH₂—CH₂—S—); 2.63 (t, 6H, CH₂—CH₂—S—, J=7.3 Hz); 3.23 (s, 6H, S—CH₂—COO); 4.27 (dd, 2H, —CHaHb-CH—CHaHb-, J=12 Hz and J=6 Hz); 4.39 (dd, 2H, —CHaHb-CH—CHaHb-, J=12 Hz and J=4.3 Hz); 5.34 (m, 1H, —CHaHb-CH—CHaHb-).

MS (MALDI-TOF): M+23=925 (M+Na⁺); M+39=941 (M+K⁺)

### Example 4b

### Preparation of 1.2,3-tri-(4-dodecylthio)butanoylglycerol

This compound was obtained according to the method described hereinabove (example 4a) from 4-(dodecylthio)butanoic acid (example 1 b) and glycerol.

Rf (dichloromethane/cyclohexane 7:3): 0.43

IR: vCO ester 1738 and 1727 cm⁻¹

NMR (¹H, CDCl₃): 0.84-0.92 (t, 9H, —CH₃, J=6.3Hz); 1.22-1.44 (multiplet, 54H, —CH₂—); 1.50-1.64 (multiplet, 6H, —CH₂—CH₂—S—CH₂—CH₂—CH₂—COO—); 1.83-1.97 (multiplet, 6H, —CH₂—S—CH₂—CH₂—CH₂—COO—); 2.42-2.59 (multiplet, 18H, —CH₂—CH₂—CH₂—S—CH₂—CH₂—CH₂—COO—); 4.11-4.20 (dd, 2H, —CHaHb-CH—CHaHb-, J=12 Hz and J=5.9 Hz); 4.29-4.36 (dd, 2H, —CHaHb-CH—CHaHb-, J=12 Hz and J=4.5 Hz); 5.22-5.32 (m, 1H, —CHaHb-CH—CHaHb-).

MS (MALDI-TOF): M+23=925 (M+Na⁺); M+39=941 (M+K⁺)

### Example 4c

### Preparation of 1.2,3-tri-(6-decylthio)hexanoylglycerol

This compound was obtained according to the method described hereinabove (example 4a) from 6-(decylthio)hexanoic acid (example 1c) and glycerol.

Rf (dichloromethane/cyclohexane 7:3): 0.43

IR: vCO ester 1730 cm⁻¹

NMR (¹H, CDCl₃): 0.85-0.92 (t, 9H, —CH₃, J=6.5 Hz); 1.21-1.50 (multiplet, 48H —CH₂—); 1.51-1.72 (multiplet, 18H, —CH₂—CH₂—S—CH₂—CH₂—CH₂—CH₂—CH₂—COO—); 2.28-2.40 (multiplet, 6H, —CH₂—S—CH₂—CH₂—CH₂—CH₂—CH₂—COO—); 2.45-2.57 (multiplet, 12H, —CH₂—S—CH₂—); 4.10-4.20 (dd, 2H, —CHaHb-CH—CHaHb-, J=12 Hz); and J=6 Hz); 4.25-4.38 (dd, 2H, —CHaHb-CH—CHaHb-, J=12 Hz and J=4.3 Hz); 5.22-5.32 (m, 1H, —CHaHb-CH—CHaHb-).

MS (MALDI-TOF): M+23=925 (M+Na⁺); M+39=941 (M+K⁺)

### Example 4d

### Preparation of 1,2,3-tritetradecylsulfoxyacetylglycerol

This compound was obtained according to the method described hereinabove (example 4a) from tetradecylsulfoxyacetic acid (example 1e) and glycerol.

Rf (dichloromethane/cyclohexane 7:3): 0.33

IR: vCO ester 1730 cm⁻¹

NMR (¹H, CDCl₃): 0.80-0.92 (t, 9H, —CH₃, J=6.4 Hz); 1.20-1.39 (multiplet, 60H, —CH₂—); 1.40-1.55 (multiplet, 6H, CH₂—); 1.70-1.90 (quint, 6H, —CH₂—CH₂—S—); 2.82-2.89 (m, 6H, —CH₂—CH₂—SO—CH₂—COO—); 3.49-3.90 (m, 6H, —CH₂—SO—CH₂—COO); 4.10-4.30 (m, 2H, —CH₂—CH—CH₂—); 4.30-4.60 (m, 2H, —CH₂—CH—CH₂—); 5.45 (m, 1H, —CH₂—CH—CH₂—).

MS (MALDI-TOF): M+1=951; M+23=974 (M+Na⁺); M+39=990 (M+K⁺)

### Example 4e

### Preparation of 1,2,3-tri-(tetradecylsulfonyl)acetylglycerol

This compound was obtained according to the method described hereinabove (example 4a) from tetradecylsulfonylacetic acid (example 1g) and glycerol.

Rf (dichloromethane/ethyl acetate 9:1 ): 0.51

MP: 107.0-110.6° C.

IR: vCO ester 1769, 1754 and 1735 cm⁻¹; vSO 1120 cm⁻¹

NMR (¹H, CDCl₃): 0.87 (t, 9H, —CH₃, J=6.5 Hz); 1.19-1.35 (multiplet, 60H, —CH₂—); 1.44-1.49 (m, 6H, —CH₂—CH₂—CH₂—SO₂—); 1.81-1.92 (m, 6H, —CH₂—CH₂—SO₂—); 3.23 (t, 6H, —CH₂—CH₂—SO₂—CH₂—COO—, J=7.5 Hz); 4.01 (s, 4H, —CH₂—SO₂—CH₂—COO); 4.03 (s, 2H, —CH₂—SO₂—CH₂—COO—); 4.67 (m, 4H, —CH₂—CH—CH₂—); 5.49 (m, 1H, —CH₂—CH—CH₂—).

MS (MALDI-TOF): M+23=1021 (M+Na⁺); M+39=1037 (M+K⁺)

### Example 4f

### Preparation of 1,2,3-tri-tetradecylselenoacetylglycerol

This compound was obtained according to the method described hereinabove (example 4a) from tetradecylselenoacetic acid (example 1d) and glycerol.

Rf (dichloromethane/cyclohexane 7:3): 0.74

IR: vCO ester 1737 and 1721 cm⁻¹

NMR (¹H, CDCl₃): 0.85-0.92 (t, 9H, —CH₃, J=6.2 Hz); 1.23-1.46 (multiplet, 66H, —CH₂—); 1.62-1.76 (multiplet, 6H, —CH₂—CH₂—CH₂—Se—); 2.72-2.79 (t, 6H, CH₂—CH₂—Se—, J=7.4 Hz); 3.15 (s, 6H, Se—CH₂—COO—); 4.10-4.30 (m, 2H, —CH₂—CH—CH₂—); 4.30-4.60 (m, 2H, —CH₂—CH—CH₂—); 5.37 (m,1 H, —CH₂—CH—CH₂—).

### Example 4a

### Preparation of 1,3-dipalmitoyl-2-tetradecylthioacetylglycerol

1,3-dipalmitoylglycerol (example 3a) (5.64 g, 9.9 mmol, 1 eq), tetradecylthioacetic acid (example 1a) (5.74 g, 19.8 mmol, 2 eq), dicyclohexylcarbodiimide (4.1 g, 19.8 mmol, 2 eq) and dimethylaminopyridine (2.42 g, 19.8 mmol, 2 eq) were dissolved in dichloromethane. The reaction mixture was stirred at room temperature for 3 days. The dicyclohexylurea which formed was filtered and washed several times with dichloromethane. The filtrate was dried. The residual product was purified by silica gel chromatography (eluent: dichloromethane/cyclohexane 4:6).

Yield: 80%

Rf (dichloromethane/cyclohexane 7:3): 0.32

MP: 60-62° C.

IR: vCO ester 1744 and 1730 cm⁻¹

NMR (¹H, CDCl₃): 0.86-0.91 (t, 9H, —CH₃, J=6.6 Hz); 1.10-1.45 (multiplet, 70H, —CH₂—); 1.57-1.64 (multiplet, 6H, —CH₂—CH₂—CH₂—S— and OCOCH₂—CH₂); 2.30-2.35 (t, 4H, OCOCH₂—CH₂—, J=7.4 Hz); 2.60-2.66 (t, 2H, CH₂—CH₂—S—, J=7.4 Hz); 3.23 (s, 2H, S—CH₂—COO); 4.14-4.21 (dd, 2H, —CHaHb-CH—CHaHb-, J=12 Hz and J=5.8 Hz); 4.30-4.36 (dd, 2H, —CHaHb-CH—CHaHb-, J=12Hz and J=4 Hz); 5.26-5.33 (m,1 H, —CHaHb-CH—CHaHb-).

MS (MALDI-TOF): M+23=861 (M+Na⁺); M+39=877 (M+K⁺)

### Example 4h

### Preparation of 1,3-dilinoleoyl-2-tetradecylthioacetylglycerol

This compound was obtained according to the method described hereinabove (example 4g) from 1,3-dilinoleoylglycerol (example 3b) and tetradecylthioacetic acid (example 1a). The product was obtained as a colorless, viscous oil.

Yield: 56%

Rf (dichloromethane/cyclohexane 7:3): 0.32

IR: vCO ester 1745 cm⁻¹

NMR (¹H, CDCl₃): 0.82-0.93 (t, 9H, —CH₃, J=6.6 Hz); 1.15-1.45 (multiplet, 50H, —CH₂—); 1.52-1.70 (multiplet, 6H, —CH₂—CH₂—CH₂—S— and OCOCH₂—CH₂); 1.93-2.14 (multiplet, 8H, —CH₂—CH═CH—CH₂—); 2.28-2.37 (t, 4H, OCOCH₂—CH₂—, J=7.5 Hz); 2.59-2.67 (t, 2H, CH₂—CH₂—S—, J=7.4 Hz); 2.70-2.83 (t, 4H, —CH₂—CH═CH—CH₂—CH═CH—CH₂—); 3.22 (s, 2H, S—CH₂—COO—); 4.12-4.23 (dd, 2H, —CHaHb-CH—CHaHb-, J=12 Hz and J=6.2 Hz); 4.28-4.37 (dd, 2H, —CHaHb-CH—CHaHb-, J=12 Hz and J=4 Hz); 5.24-5.45 (m, 1H, —CHaHb-CH—CHaHb-).

MS (MALDI-TOF): M+23=909 (M+Na⁺); M+39=925 (M+K⁺)

### Example 4i

### Preparation of 1,3-distearoyl-2-tetradecylthioacetylglycerol

This compound was obtained according to the method described hereinabove (example 4g) from 1,3-distearoylglycerol (example 3c) and tetradecylthioacetic acid (example 1a).

Yield: 41%

Rf (dichloromethane): 0.32

IR: vCO ester 1744 and 1731 cm⁻¹

NMR (¹H, CDCl₃): 0.86-0.91 (t, 9H, —CH₃, J=6.6 Hz); 1.10-1.45 (multiplet, 78H, —CH₂—); 1.57-1.64 (multiplet, 6H, —CH₂—CH₂—CH₂—S— and OCOCH₂—CH₂); 2.29-2.35 (t, 4H, OCOCH₂—CH₂—, J=7.4 Hz); 2.60-2.66 (t, 2H, CH₂—CH₂—S—, J=7.4 Hz); 3.23 (s, 2H, S—CH₂—COOH); 4.14-4.21 (dd, 2H, —CHaHb-CH—CHaHb-, J=12 Hz and J=5.8 Hz); 4.30-4.36 (dd, 2H, —CHaHb-CH—CHaHb-, J=12 Hz and J=4 Hz); 5.26-5.32 (m, 1H, —CHaHb-CH—CHaHb-).

### Example 4i

### Preparation of 1,3-oleoyl-2-tetradecylthioacetylglycerol

This compound was obtained according to the method described hereinabove (example 4g) from 1,3-dioleoylglycerol (example 3d) and tetradecylthioacetic acid (example 1 a). The product was obtained as a colorless, viscous oil.

Yield: 32%

Rf (dichloromethane/cyclohexane 7:3): 0.50

IR: vCO ester 1746 cm⁻¹

NMR (¹H, CDCl₃): 0.89 (t, 9H, —CH₃, J=6.4 Hz); 1.31 (multiplet, 66H, —CH₂—); 1.60 (multiplet, 6H, —CH₂—CH₂—CH₂—S— and OCOCH₂—CH₂—); 2.02 (multiplet, 8H, —CH₂—CH═CH—CH₂—); 2.33 (t, 4H, OCOCH₂—CH₂—, J=7.3 Hz); 2.63 (t, 2H, CH₂—CH₂—S—, J=7.7 Hz); 3.23 (s, 2H, S—CH₂—COO—); 4.18 (dd, 2H, —CHaHb-CH—CHaHb-, J=12.4 Hz and J=6.4 Hz); 4.33 (dd, 2H, —CHaHb-CH—CHaHb-, J=12.4 Hz and J=4.5 Hz); 5.33 (multiplet, 1H, —CHaHb-CH—CHaHb- and —CH₂—CH═CH—CH₂—).

MS (MALDI-TOF): M+23=913 (M+Na⁺); M+39=929 (M+K⁺)

### Example4k

### Preparation of 1,3-ditetradecanoyl-2-tetradecylthioacetylglycerol

This compound was obtained according to the method described hereinabove (example 4g) from 1,3-ditetradecanoylglycerol (example 3e) and tetradecylthioacetic acid (example 1a).

Yield: 28%

Rf (dichloromethane/cyclohexane 7:3): 0.30

MP: 60-62° C.

IR: vCO ester 1744 and 1730 cm⁻¹

NMR (¹H, CDCl₃): 0.87 (t, 9H, —CH₃, J=7.2 Hz); 1.27 (multiplet, 62H, —CH₂—); 1.60 (multiplet, 6H, —CH₂—CH₂—CH₂—S— and OCOCH₂—CH₂); 2.33 (t, 4H, OCOCH₂—CH₂—, J=7.7 Hz); 2.63 (t, 2H, CH₂—CH₂—S—, J=7.2 Hz); 3.23 (s, 2H, S—CH₂—COO); 4.18 (dd, 2H, —CHaHb-CH—CHaHb-, J=12 Hz and J=5.8 Hz); 4.33 (dd, 2H, —CHaHb-CH—CHaHb-, J=11.5 Hz and J=5.8 Hz); 5.30 (m, 1H, —CHaHb-CH—CHaHb-).

MS (MALDI-TOF): M+23=805 (M+Na⁺)

### Example 4l

### Preparation of 1-palmitoyl-2,3-ditetradecylthioacetylglycerol

Glycerol 1-palmitate (example 2b) (4.804 g, 14 mmol) was dissolved in dichloromethane (300 ml). Dicyclohexylcarbodiimide (7.498 g, 36 mmol), dimethylaminopyridine (4.439 g, 36 mmol) and tetradecylthioacetic acid (example 1a) (8.386 g, 29 mmol) were then added. The reaction mixture was stirred at room temperature for 48 hours. The dicyclohexylurea precipitate was filtered and washed with dichloromethane. The filtrate was dried. The residue was purified by silica gel chromatography (eluent:dichloromethane/cyclohexane 4:6) to give the desired compound as a white powder.

Yield: 42%

Rf (dichloromethane/cyclohexane 7:3): 0.31

MP: 57-59° C.

IR: vCO ester 1736 et 1722 cm⁻¹

NMR (¹H, CDCl₃): 0.89 (t, 9H, —CH₃, J=6.6 Hz); 1.27 (multiplet, 68H, —CH₂—); 1.60 (multiplet, 6H, —CH₂—CH₂—CH₂—S— and —OCOCH₂—CH₂); 2.33 (t, 2H, OCOCH₂—CH₂—, J=7 Hz); 2.63 (t, 4H, CH₂—CH₂—S—, J=8.9 Hz); 3.23 (s, 4H, S—CH₂—COO—); 4.23 (m, 2H, —CHaHb-CH—CHaHb-); 4.37 (m, 2H, —CHaHb-CH—CHaHb); 5.31 (m, 1H, —CHaHb-CH—CHaHb-).

MS (MALDI-TOF): M+23=893 (M+Na⁺); M+39=909 (M+K⁺)

### Example 4m

### Preparation of 1-oleoyl-3-palmitoyl-2-tetradecylthioacetylglycerol

1-oleoyl-3-palmitoylglycerol (example 3g) (2 g, 3 mmol) was dissolved in dichloromethane (150 ml). Dicyclohexylcarbodiimide (1.040 g, 5 mmol), dimethylaminopyridine (0.616 g, 5 mmol) and tetradecylthioacetic acid (example 1a) (1.455 g, 5 mmol) were then added. The mixture was stirred at room temperature for 24 hours. The dicyclohexylurea precipitate was filtered, washed with dichloromethane and the filtrate was concentrated. The residue obtained was purified by silica gel chromatography (eluent:dichloromethane/cyclohexane 4:6) to give the desired compound as an oil.

Yield: 49%

Rf (dichloromethane/cyclohexane 7:3): 0.45

MP<4° C.

IR: vCO ester 1742 cm⁻¹

NMR (¹H, CDCl₃): 0.89 (t, 9H, —CH₃, J=6.5 Hz); 1.26 (multiplet, 66H, —CH₂—); 1.60 (multiplet, 6H, —CH₂—CH₂—CH₂—S— and OCOCH₂—CH₂); 2.03 (multiplet, 4H, —CH₂—CH═CH—CH₂—); 2.33 (t, 4H, OCOCH₂—CH₂—, J=7.4 Hz); 2.63 (t, 2H, CH₂—CH₂—S—, J=7.4 Hz); 3.23 (s, 2H, S—CH₂—COO); 4.18 (dd, 2H, —CHaHb-CH—CHaHb-, J=12.2 Hz and J=6.1 Hz); 4.33 (dd, 2H, —CHaHb-CH—CHaHb-, J=12.2 Hz and J=4.4 Hz); 5.32 (multiplet, 3H, —CHaHb-CH—CHaHb- and —CH₂—CH═CH—CH₂—).

MS (MALDI-TOF): M+23=887 (M+Na⁺); M+39=903 (M+K⁺)

### Example 4n

### Preparation of 1,3-dipalmitoyl-2-docosylthioacetylglycerol

This compound was obtained according to the method described hereinabove (example 4g) from 1,3-dipalmitoylglycerol (example 3a) and docosylthioacetic acid (example 1i).

Yield: 77%

Rf (dichloromethane/cyclohexane 7:3): 0.32

IR: vCO ester 1745 and 1730 cm⁻¹

NMR (¹H, CDCl₃): 0.86-0.91 (t, 9H, —CH₃, J=6.6 Hz); 1.10-1.45 (multiplet, 86H, —CH₂—); 1.57-1.64 (multiplet, 6H, —CH₂—CH₂—CH₂—S— and OCOCH₂—CH₂); 2.29-2.34 (t, 4H, OCOCH₂—CH₂—, J=7.5 Hz); 2.60-2.66 (t, 2H, CH₂—CH₂—S—, J=7.4 Hz); 3.23 (s, 2H, S—CH₂—COO—); 4.13-4.22 (dd, 2H, —CHaHb-CH—CHaHb-, J=12 Hz and J=5.8 Hz); 4.30-4.36 (dd, 2H, —CHaHb-CH—CHaHb-, J=12 Hz and J=4 Hz); 5.27-5.34 (m, 1H, —CHaHb-CH—CHaHb-).

### Example 4o

### Preparation of 1.3-ditetradecylthioacetyl-2-palmitoyl-glycerol

This compound was obtained according to the method described hereinabove (example 4g) from 1,3-ditetradecylthioacetylglycerol (example 3f) and palmitic acid.

Yield: 36%

MP: 59-61° C.

Rf (dichloromethane/cyclohexane 7:3): 0.35

IR: vCO ester 1740 cm⁻¹

NMR (¹H, CDCl₃): 0.89 (t, 9H, —CH₃, J=6.56 Hz); 1.26 (multiplet, 68H, —CH₂—); 1.55-1.65 (multiplet, 6H, —CH₂—CH₂—CH₂—S— and —OCOCH₂—CH₂—); 2.34 (td, 2H, OCOCH₂—CH₂—, J=7.7 Hz and J=1.9 Hz); 2.63 (td, 4H, CH₂—CH₂—S—, J=7.3 Hz and J=1.9 Hz); 3.23 (s, 4H, S—CH₂—COO—); 3.68 (dd, 2H, —CHaHb-CH—CHaHb-, J=10.4 Hz and J=4.6 Hz); 4.36 (dd, 2H, —CHaHb-CH—CHaHb-, J=11.9 Hz and J=4.2 Hz); 5.31 (m, 1H, —CHaHb-CH—CHaHb-).

MS (MALDI-TOF): M+23=893 (M+Na⁺); M+39=909 (M+K⁺)

### Example 4p

### Preparation of 1,3-diacetyl-2-tetradecylthioacetylglycerol

This compound was obtained according to the method described hereinabove (example 4g) from 1,3-diacetylglycerol (example 3h) and tetradecylthioacetic acid (example 1a).

Yield: 10%

Rf (ethyl acetate/cyclohexane 3:7): 0.47

MP<4° C.

IR: vCO ester 1748 cm⁻¹

NMR (¹H, CDCl₃): 0.89 (t, 9H, —CH₃, J=6.9 Hz); 1.26 (multiplet, 20H, —CH₂—); 1.60 (multiplet, 4H, —CH₂—CH₂—CH₂—S—); 2.09 (s, 6H, —OCOCH₃); 2.64 (t, 2H, CH₂—CH₂—S—, J=7.4 Hz); 3.24 (s, 2H, S—CH₂—COO); 4.17 (dd, 2H, —CHaHb-CH—CHaHb-, J=12 Hz and J=5.8 Hz); 4.34 (dd, 2H, —CHaHb-CH—CHaHb-, J=12 Hz and J=4 Hz); 5.28 (m, 1H, —CHaHb-CH—CHaHb-).

MS (MALDI-TOF): M+23=469 (M+Na⁺); M+39=485 (M+K⁺)

### Example 4g

### Preparation of 1,3-dioctanoyl-2-tetradecylthioacetylglycerol

This compound was obtained according to the method described hereinabove (example 4g) from 1,3-dioctanoylglycerol (example 3i) and tetradecylthioacetic acid (example 1a).

Yield: 88%

Rf (dichloromethane 10): 0.52

MP<4° C.

IR: vCO ester 1745 cm⁻¹

NMR (¹H, CDCl₃): 0.89 (t, 9H, —CH₃, J=7.0 Hz); 1.27 (multiplet, 38H, —CH₂—); 1.60 (multiplet, 6H, —CH₂—CH₂—CH₂—S— and OCOCH₂—CH₂); 2.32 (t, 4H, OCOCH₂—CH₂—, J=7.3 Hz); 2.63 (t, 2H, CH₂—CH₂—S—, J=7.3 Hz); 3.23 (s, 2H, S—CH₂—COO); 4.17 (dd, 2H, —CHaHb-CH—CHaHb-, J=11.9 Hz and J=5.8 Hz); 4.33 (dd, 2H, —CHaHb-CH—CHaHb-, J=11.9 Hz and J=4.3 Hz); 5.30 (m, 1H, —CHaHb-CH—CHaHb-).

MS (MALDI-TOF): M+23=637 (M+Na⁺); M+39=653 (M+K⁺)

### Example 4r

### Preparation of 1,3-diundecanoyl-2-tetradecylthioacetylglycerol

This compound was obtained according to the method described hereinabove (example 4g) from 1,3-diundecanoylglycerol (example 3j) and tetradecylthioacetic acid (example 1a).

Yield: 28%

Rf (dichloromethane/cyclohexane 7:3): 0.16

IR: vCO ester 1738 and 1725 cm⁻¹

NMR (¹H, CDCl₃): 0.89 (t, 9H, —CH₃, J=6.9 Hz); 1.26 (multiplet, 50H, —CH₂—); 1.62 (multiplet, 6H, —CH₂—CH₂—CH₂—S— and OCOCH₂—CH₂); 2.33 (t, 4H, OCOCH₂—CH₂—, J=7.7 Hz); 2.63 (t, 2H, CH₂—CH₂—S—, J=7.3 Hz); 3.23 (s, 2H, S—CH₂—COO); 4.20 (dd, 2H, —CHaHb-CH—CHaHb-, J=12.1 Hz and J=6.1 Hz); 4.35 (dd, 2H, —CHaHb-CH—CHaHb-, J=12.1 Hz and J=4.5 Hz); 5.29 (m, 1H, —CHaHb-CH—CHaHb-).

MS (MALDI-TOF): M+23=722 (M+Na⁺); M+39=738 (M+K⁺)

### Example 5

### Preparation of 2-aminoglycerol Derivatives

### Example 5a

### Preparation of 2-tetradecylthioacetamidopropane-1.3-diol

Tetradecylthioacetic acid (example la) (2.878 g, 0.010 mol) and 2-amino-1,3-propanediol (1 g, 0.011 mol) were placed in a flask and heated at 190° C. for 1 hour. After cooling to room temperature, the medium was taken up in chloroform and washed with water. The organic phase was dried on magnesium sulfate, filtered then evaporated to form a solid ochre residue. This residue was stirred in diethyl ether for 12 hours. The product was isolated by filtration in the form of a white powder.

Yield: 6%

Rf (dichloromethane/methanol 9:1): 0.60

MP: 95-97° C.

IR: vCO amide 1640 cm⁻¹

NMR (¹H, CDCl₃): 0.84-0.93 (t, 3H, —CH₃, J=6.4 Hz); 1.21-1.45 (multiplet, 22H, —CH₂—); 1.54-1.72 (m, 2H, —CH₂—CH₂—CH₂—S—); 2.52-2.59 (t, 2H, CH₂—CH₂—S—, J=7.1 Hz); 2.63 (sl, 2H, OH); 3.27 (s, 2H, S—CH₂—COO); 3.77-3.96 (multiplet, 4H, —CH₂—CH—CH₂—); 3.97-4.04 (m, 1H, —CH₂—CH—CH₂—); 7.55 (d, 1H, —CONH—, J=6.7 Hz).

MS (MALDI-TOF): M+1=362; M+23=384 (M+Na⁺); M+39=400 (M+K⁺)

### Example 5b

### Preparation of 2-tetradecylthioacetamido-1,3-ditetradecylthioacetyloxypropane

2-tetradecylthioacetamidopropan-1,3-diol (example 5a) (1 g, 2.77 mmol) was dissolved in dichloromethane (180 ml). Dicyclohexycarbodiimide (1.427 g, 6.91 mmol), dimethylaminopyridine (0.845 g, 6.91 mmol) and tetradecylthioacetic acid (example 1a) (1.995 g, 6.91 mmol) were then added. The reaction mixture was stirred at room temperature for 48 hours. The dicyclohexylurea precipitate was filtered and washed with dichloromethane and the filtrate was concentrated. The residue obtained was purified by silica gel chromatography (eluent:dichloromethane/cyclohexane 7:3). The desired compound was obtained as a white powder.

Yield: 66%

Rf (dichloromethane): 0.18

MP: 82-84° C.

IR: vCO ester 1715 and 1730 cm⁻¹; vCO amide 1648 cm⁻¹

NMR (¹H, CDCl₃): 0.84-0.95 (t, 9H, —CH₃, J=6.6 Hz); 1.22-1.45 (multiplet, 66H —CH₂—); 1.54-1.69 (multiplet, 6H, —CH₂—CH₂—CH₂—S—); 2.48-2.55 (t, 2H, CH₂—CH₂—S—CH₂—CONH—, J=7.5 Hz); 2.59-2.70 (t, 4H, CH₂—CH₂—S—CH₂—COO—, J=7.2Hz); 3.24 (s, 6H, S—CH₂—CO—); 4.18-4.35 (multiplet, 4H, —CH₂—CH—CH₂—); 4.47-4.60 (m, 1H, —CH₂—CH—CH₂—); 7.23 (d, 1H, —CONH—, J=8.5 Hz).

MS (MALDI-TOF): M+23=924 (M+Na⁺)

### Example 6

### Preparation of 2-thioalycerol Derivatives

### Example 6a

### Preparation of 2-(tetradecylthio)thiolacetic Acid

### Preparation of S-triphenylmethyl 2-(tetradecylthio)thioacetate

Triphenylmethylthiol (9.58 g, 35 mmol) was dissolved in dichloromethane, and dicyclohexylcarbodiimide (7.15 g, 35 mmol), dimethylaminopyridine (4.24 g, 35 mmol) and tetradecylthioacetic acid (example 1a) (10 g, 35 mmol) were then added. The reaction mixture was stirred at room temperature for 24 hours. The dicyclohexylcarbodiimide was filtered and washed with dichloromethane. The filtrate was dried. The residue was purified by silica gel chromatography (eluent:dichloromethane/cyclohexane 1:9).

Yield: 30%

Rf (dichloromethane/cyclohexane 2:8): 0.43

MP: 45-50° C.

IR: vCO ester 1689 cm⁻¹

NMR (¹H, CDCl₃): 0.89 (t, 3H, —CH₃, J=6.4 Hz); 1.26 (multiplet, 22H, —CH₂—); 1.51-1.54 (m, 2H, —CH₂—CH₂—CH₂—S—); 2.47 (t, 2H, CH₂—CH₂—S—CH₂—COS—, J=7.1 Hz); 3.30 (s, 2H, S—CH₂—COS—); 7.23 (multiplet, 15H, aromatic H).

### Preparation of 2-(tetradecylthio)thiolacetic Acid

S-triphenylmethyl 2-(tetradecylthio)thioacetate (4.715 g, 9 mmol) was added in the cold to a suspension of mercuric acetate (5.495 g, 17 mmol) in dichloromethane (150 ml). The reaction mixture was stirred for 18 hours, then filtered on Celite® and washed with hot dichloromethane. The filtrate was evaporated to give a powdery residue which was taken up in absolute ethanol and filtered. Concentration of the filtrate yielded a yellow oil which was used without further purification.

Rf (dichloromethane/methanol 9:1): 0.58

### Example 6b

### Preparation of 2-iodo-1,3-ditetradecylthioacetoxypropane

1,3-ditetradecylthioacetylglycerol (example 3f) (2 g, 3 mmol) was dissolved in toluene (180 ml). Imidazole (0.538 g, 8 mmol), triphenylphosphine (2.072 g, 8 mmol) and iodine (1.604 g, 6 mmol) were then added. The reaction mixture was stirred at room temperature. After 20 hours of reaction, a saturated sodium sulfite solution was added until complete blanching of the medium. The medium was allowed to settle and the aqueous phase was extracted with toluene. The organic phases were combined and washed with a saturated aqueous sodium chloride solution. The organic phase was dried on magnesium sulfate, filtered and the solvent was evaporated. The residue (4.4 g) was purified by chromatography on a Puriflash column (eluent:dichloromethane/cyclohexane 1:9 then 3:7).

Yield: 95%

Rf (dichloromethane/cyclohexane 6:4): 0.62

MP: 51-53° C.

NMR (¹H, CDCl₃): 0.89 (t, 6H, —CH₃, J=6.6 Hz); 1.27 (multiplet, 44H, —CH₂—); 1.63 (multiplet, 4H, —CH₂—CH₂—CH₂—S—); 2.66 (t, 4H, CH₂—CH₂—S—CH₂—COO—, J=7.4 Hz); 3.26 (s, 4H, S—CH₂—CO—); 4.42 (multiplet, 5H—CH₂—CH—CH₂—)).

MS (MALDI-TOF): M+23=765 (M+Na⁺); 781 (M+K⁺)

### Example 6c

### Preparation of 1,3-ditetradecylthioacetoxy-2-(tetradecylthiomethyl)carbonylthiopropane

2-iodo-1,3-ditetradecylthioacetoxypropane (example 6b) (200 mg, 0.27 mmol) and 2-(tetradecylthio)thiolacetic acid (example 6a) (82 mg, 0.27 mmol) were dissolved in distilled tetrahydrofuran (30 ml). The reaction mixture was cooled in an ice bath before adding soduim hydride (22 mg, 0.54 mmol). The mixture was stirred at room temperature for 48 hours, then the sodium hydride was hydrolyzed and the tetrahydrofuran evaporated. The medium was extracted with ethyl acetate; the organic phase was washed with water, dried on magnesium sulfate, filtered and evaporated. The resulting oily yellow residue (164 mg) was purified by silica gel chromatography on a short column (eluent:dichloromethane/cyclohexane 5:5) to give the desired compound as a yellow oil.

Rf (dichloromethane/cyclohexane 5:5): 0.20

IR: vCO ester 1737 cm⁻¹

NMR (¹H, CDCl₃): 0.87 (t, 9H, —CH₃, J=6.7 Hz); 1.26 (multiplet, 66H, —CH₂—); 1.56-1.63 (multiplet, 6H, —CH₂—CH₂—CH₂—S—); 2.19 (s, 2H, S—CH₂—COS—); 2.65 (t, 4H, CH₂—CH₂—S—CH₂—COO—, J=7.5 Hz); 2.87 (t, 2H, CH₂—CH₂—S—CH₂—COS—, J=4.6 Hz); 3.22-3.26 (m, 1H, —CH₂—CH—CH₂—); 3.27 (s, 4H, S—CH₂—COO—); 3.97-4.02 (m, 2H, —CHaHb-CH—CHaHb-); 4.46-4.51 (m, 2H, —CHaHb-CH—CHaHb-).

MS (MALDI-TOF): M+1=919 (M+H⁺)

### Example 7

### Preparation of 3-(tetradecylthioacetylamino)propane-1,2-diol

Tetradecylthioacetic acid (example 1a) (14.393 g, 50 mmol) and 3-amino-propane-1,2-diol (5 g, 55 mmol) were placed in a flask and heated at 190° C. for 1 hour. The reaction mixture was cooled to room temperature, taken up in chloroform and washed once with water. The organic phase was dried on magnesium sulfuate, filtered and dried. The residue was stirred in ether and the product was isolated by filtration.

Yield: 22%

Rf (dichloromethane/methanol 9:1): 0.60

MP: 89-92° C.

IR: vNH and OH 3282 cm⁻¹; vCO amide 1640 cm⁻¹

NMR (¹H, CDCl₃): 0.89 (t, 3H, —CH₃, J=6.5 Hz); 1.26 (multiplet, 22H, —CH₂—); 1.57 (m, 2H, —CH₂—CH₂—S—); 2.54 (t, 2H, —CH₂—CH₂—S—, J=7.6 Hz); 3.27 (s, 2H, S—CH₂—CONH—); 3.47 (m, 2H, —CONH—CH₂—CHOH—CH₂OH); 3.58 (m, 1H, —CONH—CH₂—CHOH—CH₂OH);3.81 (m, 2H, —CONH—CH₂—CHOH—CH₂OH); 7.33 (sl, 1H, —CONH).

MS (MALDI-TOF): M+1=362 (M+H); M+23=385 (M+Na⁺); M+39=400 (M+K⁺)

### Example 8

### 3-(palmitovyamino)propane-1,2-diol

This compound was synthesized according to the method described hereinabove (example 7) from 3-aminopropane-1,2-diol and palmitic acid.

Yield: 86%

Rf (dichloromethane/methanol 9:1): 0.50

IR: vNH and OH 3312 cm⁻¹; vCO amide 1633 cm⁻¹

MP: 104-108° C.

NMR (¹H, CDCl₃): 0.89 (t, 3H, —CH₃, J=6.5 Hz); 1.28 (multiplet, 24H, —CH₂—); 1.64 (m, 2H, —CH₂—CH₂—CO—); 2.24 (m, 2H, —CH₂—CH₂—CO—); 3.43 (m, 2H, —CONH—CH₂—CHOH—CH₂OH); 3.55 (m, 2H, —CONH—CH₂—CHOH—CH₂OH); 3.78 (m,1 H, —CONH—CH₂—CHOH—CH₂OH); 5.82 (sl, 1H, —CONH—).

MS (MALDI-TOF): M+1=330 (M+H)

### Example 9

### Preparation of 1,2-(dipalmitoyloxy)-3-tetradecylthioacetylaminopropane

3-(tetradecylthioacetylamino)propane-1,2-diol (example 7) (1 g, 2.77 mmol) was dissolved in dichloromethane (200 ml). Dicyclohexylcarbodiimide (1.426 g, 6.91 mmol), dimethylaminopyridine (0.845 g, 6.91 mmol) and palmitic acid (1.773 g, 6.91 mmol) were then added and the mixture was stirred at room temperature for 48 hours. The dicyclohexylurea which precipitated was filtered and washed with dichloromethane. The filtrate was vacuum evaporated. The residue was purified by chromatography on silica gel (eluent:dichloromethane/cyclohexane 6:4).

Yield: 28%

Rf (dichloromethane/cyclohexane 7:3): 0.28

MP: 73-75° C.

IR: vNH 3295 cm⁻¹; vCO ester 1730 cm⁻¹; vCO amide 1663 cm⁻¹

NMR (¹H, CDCl₃): 0.89 (t, 9H, —CH₃, J=6.5 Hz); 1.26 (multiplet, 70H, —CH₂—); 1.57 (multiplet, 6H, —CH₂—CH₂—S— and OCOCH₂—CH₂); 2.33 (t, 4H, OCOCH₂—CH₂—, J=7.3 Hz); 2.51 (t, 2H, CH₂—CH₂—S—, J=7.3 Hz); 3.22 (s, 2H, S—CH₂—CONH—); 3.47 (m, 1H, —CONH—CHaHb-CH—CHcHd-); 3.62 (m, 1H, —CONH—CHaHb-CH—CHcHd); 4.12 (dd, 1H, —CHaHb-CH—CHcHd-, J=12.1 Hz and J=5.7 Hz); 4.36 (dd, 1H, —CHaHb-CH—CHcHd-, J=12.1 Hz and J=4.4 Hz); 5.15 (m, 1H, —CHaHb-CH—CHaHb); 7.20 (m, 1H, —NHCO—).

MS (MALDI-TOF): M+1=838 (M+H); M+23=860 (M+Na⁺); M+39=876 (M+K⁺)

### Example 10

### Preparation of 1,2-(ditetradecylthioacetyloxy)-3-tetradecylthioacetylaminopropane

This compound was synthesized according to the method described hereinabove (example 9) from 3-(tetradecylthioacetylamino)propane-1,2-diol (example 7) and tetradecylthioacetic acid (example 1a).

Yield: 41 %

Rf (dichloromethane): 0.23

IR: vNH 3308 cm⁻¹; vCO ester 1722 and 1730 cm⁻¹; vCO amide 1672 cm⁻¹

MP: 65-67° C.

NMR (¹H, CDCl₃): 0.89 (t, 9H, —CH₃, J=6.4 Hz); 1.26 (multiplet, 66H, —CH₂—); 1.59 (multiplet, 6H, —CH₂—CH₂—S—); 2.53 (t, 2H, —CH₂—CH₂—S—CH₂—CONH—, J=7.3 Hz); 2.64 (t, 4H, CH₂—CH₂—S—CH₂—COO—, J=7.3 Hz); 3.23 (s, 4H, S—CH₂—COO—); 3.24 (s, 2H, S—CH₂—CONH—); 3.52 (m, 1H, —CONH—CHaHb-CH—CHcHd-); 3.67 (m, 1H, —CONH—CHaHb-CH—CHcHd-); 4.22 (dd, 1H, —CHaHb-CH—CHcHd-, J=12.2 Hz and J=5.4 Hz); 4.36 (dd, 1H, —CHaHb-CH—CHcHd-, J=12.2 Hz and J=3.9 Hz); 5.19 (m, 1H, —CHaHb-CH—CHaHb-); 7.18 (m, 1H, —NHCO—).

MS (MALDI-TOF): M+1=902 (M+H); M+23=924 (M+Na⁺); M+39=940 (M+K⁺)

### Example 11

### Preparation du 1,2-(ditetradecylthioacetyloxy)-3-Palmitoylaminopropane

This compound was synthesized according to the method described hereinabove (example 9) from 3-(palmitoylamino)propane-1,2-diol (example 8) and tetradecylthioacetic acid (example 1a).

Yield: 8%

Rf (ethyl acetate/cyclohexane 2:8): 0.33

IR: vNH 3319 cm⁻¹; vCO ester 1735 cm⁻¹; vCO amide 1649 cm⁻¹

MP: 82-83° C.

NMR (¹H, CDCl₃): 0.89 (t, 9H, —CH₃, J=6.4 Hz); 1.26 (multiplet, 68H, —CH₂—); 1.60 (multiplet, 6H, —CH₂—CH₂—S— and —CH₂—CH₂—CONH—); 2.18 (t, 2H, —CH₂—CH₂—CONH—, J=6.8 Hz); 2.64 (multiplet, 4H, CH₂—CH₂—S—CH₂—COO—); 3.22 (s, 2H, —S—CH₂—COO—); 3.24 (s, 2H, —S—CH₂—COO—); 3.47 (m, 1H, —CONH-CHaHb-CH—CHcHd-); 3.62 (m, 1H, —CONH—CHaHb-CH—CHcHd-); 4.23 (dd, 1H, —CHaHb-CH—CHcHd-, J=11.9 Hz and J=5.6 Hz); 4.36 (dd, 1H, —CHaHb-CH—CHcHd-, J=12.2 Hz and J=4 Hz); 5.15 (m, 1H, —CHaHb-CH—CHaHb-); 5.85 (m, 1H, —NHCO—).

MS (MALDI-TOF): M+1=870 (M+H)

### Example 12

### Preparation of 1,3-di(oleoylamino)propan-2-ol

Oleic acid (5.698 g, 20 mmol) and 1,3-diaminopropan-2-ol (1 g, 11 mmol) were placed in a flask and heated at 190° C. for 2 hours. The reaction mixture was cooled to room temperature, then taken up in chloroform and washed with water. The aqueous phase was extracted with chloroform and the organic phases were combined, dried on magnesium sulfate, filtered and evaporated to dryness to yield an oily black residue (6.64 g) which was purified by chromatography on silica gel (eluent:dichloromethane/methanol 99:1). The resulting product was then washed with ether and filtered.

Yield: 23%

Rf (dichloromethane/methanol 95:5): 0.43

IR: vNH 3306 cm⁻¹; vCO amide 1646 and 1630 cm^{-1 MP:} 88-92° C.

NMR (¹H, CDCl₃): 0.89 (t, 6H, —CH₃, J=6.2 Hz); 1.28 (multiplet, 68H, —CH₂—); 1.61-1.66 (multiplet, 4H, —CH₂—CH₂—CONH—); 1.98-2.02 (multiplet, 8H, —CH₂—CH═CH—CH₂—); 2.23 (t, 4H, —CH₂—CH₂—CONH—, J=7.0 Hz); 3.25-3.42 (multiplet, 4H, —CONH—CH₂—CH—CH₂—); 3.73-3.80 (m, 1H, —CONH—CH₂—CH—CH₂—); 5.30-5.41 (multiplet, 4H, —CH₂—CH═CH—CH₂—); 6.36 (multiplet, 2H, —NHCO—).

MS (MALDI-TOF): M+1=619 (M+H⁺); M+23=641 (M+Na⁺); M+39=657 (M+K⁺)

### Example 13

### Preparation of 1,3-di(tetradecylthioacetylamino)Propan-2-ol

This compound was synthesized according to the method described hereinabove (example 12) from 1,3-diaminopropan-2-ol and tetradecylthioacetic acid (example 1a).

Yield: 94%

Rf (dichloromethane/methanol 95:5): 0.44

IR: vNH 3275 cm⁻¹; vCO amide 1660 and 1633 cm⁻¹

MP: 101-104° C.

NMR (¹H, CDCl₃): 0.89 (t, 6H, —CH₃, J=6.3 Hz); 1.28 (multiplet, 44H, —CH₂—); 1.57-1.62 (multiplet, 4H, —CH₂—CH₂—S—CH₂—CONH—); 2.55 (t, 4H, —CH₂—CH₂—S—CH₂—CONH—, J=7.2 Hz); 3.26 (s, 4H, —S—CH₂—CONH—); 3.32-3.36 (multiplet, 2H, —CONH—CHₐH_{b}—CH—CHₐH_{b}—NHCO—); 3.43-3.49 (multiplet, 2H, —CONH—CHₐH_{b}—CH—CHₐH_{b}—NHCO—); 3.82-3.84 (m, 1H, —CONH—CH₂—CH—CH₂—NHCO—); 7.44 (sl, 2H, —NHCO).

MS (MALDI-TOF): M+23=653 (M+Na⁺); M+39=669 (M+K⁺)

### Example 14

### Preparation of 1,3-di(stearoylamino)propan-2-ol

This compound was synthesized according to the method described hereinabove (example 12) from 1,3-diaminopropan-2-ol and stearic acid.

Yield: 73%

Rf (dichloromethane/methanol 95:5): 0.28

IR: vNH 3306 cm⁻¹; vCO amide 1647 and 1630 cm⁻¹

MP: 123-130° C.

MS (MALDI-TOF): M+23=645 (M+Na⁺)

### Example 15

### Preparation of 1,3-diamino-2-(tetradecylthioacetyloxy)propane dihydrochloride

### Preparation of 1,3-di(tert-butyloxycarbonylamino)propan-2-ol (example 15a)

1,3-diaminopropan-2-ol (3 g, 0.033 mol) was dissolved in methanol (300 ml) followed by the addition of triethylamine (33 ml dropwise) and di-tert-butyl dicarbonate [(BOC)₂O] (wherein BOC corresponds to tertbutyloxycarbonyl) (21.793 g, 0.100 mol). The reaction medium was heated at 40-50° C. for 20 min then stirred at room temperature for 1 hour. After evaporation of the solvent, the colorless oil residue was purified by chromatography on silica gel (eluent:dichloromethane/methanol 95:5). The reaction yielded a colorless oil which crystallized slowly.

Yield: quantitative

Rf (dichloromethane/methanol 95:5): 0.70

IR: vNH 3368 cm⁻¹; vCO carbamate 1690 cm⁻¹

MP: 98-100° C.

NMR (¹H, CDCl₃): 1.45 (multiplet, 18H, —CH₃— (BOC)); 3.02 (sl, 1H, OH); 3.15-3.29 (multiplet, 4H, BOCNH—CH₂—CH—CH₂—NHBOC); 3.75 (m, 1H, BOCNH—CH₂—CH—CH₂—NHBOC); 5.16 (multiplet, 2H, —NHBOC).

MS (MALDI-TOF): M+1=291 (M+H⁺); M+23=313 (M+Na⁺); M+39=329 (M+K⁺)

### Preparation of 1,3-di(tert-butyloxycarbonylamino)-2-(tetradecylthioacetyloxy)-propane (example 15b)

1,3-(di-tert-butoxycarbonylamino)-propan-2-ol (example 15a) (1 g, 3.45 mmol), tetradecylthioacetic acid (example 1a) (0.991 g, 3.45 mmol) and dimethylaminopyridine (0.042 g, 0.34 mmol) were dissolved in dichloromethane (40 ml) at 0° C. Dicyclohexylcarbodiimide (0.709 g, 3.45 mmol) diluted in dichloromethane was then added dropwise and the mixture was stirred at 0° C. for 30 min, then brought to room temperature. After 20 hours of reaction, the dicyclohexylurea precipitate was filtered and the filtrate was dried. The oily residue was purified by chromatography on silica gel (eluent:dichloromethane/cyclohexane 5:5 followed by dichloromethane/ethyl acetate 98:2).

Yield: 52%

Rf (dichloromethane/ethyl acetate 95:5): 0.43

IR: vNH 3369 cm⁻¹; vCO carbamate 1690 cm⁻¹; vCO ester 1719 cm⁻¹

NMR (¹H, CDCl₃): 0.89 (t, 3H, CH₃, J=6.3 Hz); 1.26 (multiplet, 22 H, —CH₂—); 1.45 (multiplet, 18H, —CH₃— (BOC)); 1.56-1.66 (m, 2H, —CH₂—CH₂—S—CH₂—CO); 2.64 (t, 2H, —CH₂—CH₂—S—CH₂—CO, J=7.5 Hz); 3.20 (s, 2H, CH₂—S—CH₂—CO); 3.35 (multiplet, 4H, BOCNH—CH₂—CH—CH₂—NHBOC); 4.89 (m, 1H, BOCNH—CH₂—CH—CH₂—NHBOC); 5.04 (multiplet, 2H, —NHBOC).

MS (MALDI-TOF): M+23=583 (M+Na⁺); M+39=599 (M+K⁺)

### Preparation of 1,3-diamino-2-(tetradecylthioacetyloxy)propane dihydrochloride (example 15)

1,3-(ditert-butoxycarbonylamino)-2-tetradecylthioacetyloxypropane (example 15b) (0.800 g, 1.43 mmol) was dissolved in diethyl ether (50 ml) saturated with gaseous hydrochloric acid. The reaction medium was stirred at room temperature for 20 hours. The precipitate which formed was then filtered and washed with ether. The product was obtained as the dihydrochloride.

Yield: 88%

Rf (dichloromethane/methanol 7:3): 0.37

IR: vNH₂ 3049 and 3099 cm⁻¹; vCO ester 1724 cm⁻¹

MP: 224° C. (decomposition)

NMR (¹H, CDCl₃): 0.86 (t, 3H, CH₃, J=6.3 Hz); 1.24 (multiplet, 22 —CH₂—); 1.48-1.55 (m, 2H, —CH₂—CH₂—S—CH₂—CO); 2.57 (t, 2H, —CH₂—CH₂—S—CH₂—CO, J=7.2 Hz); 3.16 (multiplet, 4H, BOCNH—CH₂—CH—CH₂—NH); 3.56 (s, 2H, CH₂—S—CH₂—CO); 5.16 (m, 1H, BOCNH—CH₂—CH—CH₂—NH); 8.43 (multiplet, 6H, —NH₂.HCl).

MS (MALDI-TOF): M+1=361 (M+H⁺); M+23=383 (M+Na⁺); M+39=399 (M+K⁺)

### Example 16

### Preparation of 1,3-ditetradecylthioacetylamino-2-(tetradecylthioacetyloxy)propane

1,3-diamino-2-tetradecylthioacetyloxypropane dihydrochloride (example 15) (0.400 g, 0.92 mmol) and tetradecylthioacetic acid (example 1a) (0.532 g, 1.84 mmol) were dissolved in dichloromethane (50 ml) at 0° C. followed by the addition of triethylamine (0.3 ml, 2.1 mmol), dicyclohexylcarbodiimide (0.571 g, 2.77 mmol) and hydroxybenzotriazole (HOBT) (0.249 g, 1.84 mmol). The reaction medium was stirred at 0° C. for 1 hour then brought to room temperature for 48 hours. The dicyclohexylurea precipitate was filtered and washed with dichloromethane. The filtrate was vacuum evaporated. The residue obtained (1.40 g) was purified by chromatography on silica gel (eluent:dichloromethane followed by dichloromethane/ethyl acetate 9:1).

Yield: 74%

Rf (dichloromethane/ethyl acetate 8:2): 0.25

IR: vNH 3279 and 3325 cm⁻¹; vCO ester 1731 cm⁻¹; vCO amide 1647 and 1624 cm⁻¹

MP: 87-89° C.

NMR (¹H, CDCl₃): 089 (t, 9H, CH₃, J=6.6 Hz); 1.26 (multiplet, 66H, —CH₂—); 1.55-1.60 (multiplet, 6H, —CH₂—CH₂—S—CH₂—CO); 2.55 (t, 4H, —CH₂—CH₂—S—CH₂—CONH—, J=7.2 Hz); 2.65 (t, 2H, —CH₂—CH₂—S—CH₂—COO—, J=7.2Hz); 3.21 (s, 2H, —CH₂—S—CH₂—COO—); 3.25 (s, 4H, —CH₂—S—CH₂—CONH—); 3.40-3.49 (m, 2H, —CONH—CHₐH_{b}—CH—CHₐH_{b}—NHCO—); 3.52-3.61 (m, 2H, —CONH—CHₐH_{b}—CH—CHₐH_{b}—NHCO—); 4.96 (m, 1H, —CONH—CH₂—CH—CH₂—NHCO—); 7.42 (multiplet, 2H, —NHCO—).

MS (MALDI-TOF): M+1=901 (M+H⁺); M+23=923 (M+Na⁺); M+39=939 (M+K⁺)

### Example 17

### Preparation of 1,3-dioleoylamino-2-(tetradecylthioacetyloxy)propane

This compound was synthesized according to the method described in example 16 from 1,3-diamino-2-tetradecylthioacetyloxypropane dihydrochloride (example 15) and oleic acid.

Yield: 15%

Rf (dichloromethane/ethyl acetate 8:2): 0.38

IR: vNH 3325 cm⁻¹; vCO ester 1729 cm⁻¹; vCO amide 1640 and 1624 cm⁻¹

MP: 57-59° C.

NMR (¹H, CDCl₃): 0.89 (t, 9H, CH₃, J=6.6 Hz); 1.26 (multiplet, 62H, —CH₂—); 1.59-1.74 (multiplet, 6H, —CH₂—CH₂—S—CH₂—CO); 1.92-2.03 (multiplet, 8H, —CH₂—CH═CH—CH₂—); 2.22 (t, 4H, —CH₂—CH₂—S—CH₂—CONH—, J=7.2 Hz); 2.65 (t, 2H, —CH₂—CH₂—S—CH₂—COO—, J=7.4 Hz); 3.19 (s, 2H, —CH₂—S—CH₂—COO—); 3.25-3.34 (m, 2H, —CONH—CHₐH_{b}—CH—CHₐH_{b}—NHCO—); 3.56-3.65 (m, 2H, —CONH—CHₐH_{b}—CH—CHₐH_{b}—NHCO—); 4.87 (m, 1H, —CONH—CH₂—CH—CH₂—NHCO—); 5.34 (multiplet, 4H, —CH₂—CH═CH—CH₂—); 6.27 (multiplet, 2H, —NHCO—).

MS (MALDI-TOF): M+1=889 (M+H⁺); M+23=912 (M+Na⁺)

### Example 18

### Preparation of 2,3-ditetradecylthioacetylaminopropan-1-ol

### Preparation of methyl 2,3-diaminopropanoate dihydrochloride (example 18a)

2,3-diaminopropionic acid hydrochloride (1 g, 7 mmol) was dissolved in methanol (40 ml). The medium was cooled in an ice bath, followed by the addition of thionyl chloride (2.08 ml, 28 mmol). The medium was brought to room temperature then refluxed for 20 hours. The solvent was evaporated and the residue was triturated in heptane. The resulting precipitate was filtered, washed and dried to give a yellowish solid.

Yield: 94%

Rf: (dichloromethane/methanol 9:1): 0.03

IR: vNH₂ 2811 cm⁻¹; vCO ester 1756 cm⁻¹

MP: 170-180° C. (decomposition)

NMR (¹H, CDCl₃): 3.78 (s, 3H, —CH₃); 4.33 (m, 3H, —CH₂— et —CH—); 8.77 (m, 3H, —NH₂.HCl); 9.12 (m, 3H, —NH₂.HCl).

### Preparation of methyl 2,3-ditetradecylthioacetylaminopropanoate (example 18b)

Methyl 2,3-diaminopropanoate dihydrochloride (example 18a) (0.500 g, 2.62 mmol) and tetradecylthioacetic acid (example 1a) (1.51 g, 5.23 mmol) were dissolved in dichloromethane (80 ml) at 0° C. followed by the addition of triethylamine (0.79 ml), dicyclohexylcarbodiimide (1.62 g, 7.85 mmol) and hydroxybenzotriazole (0.707 g, 5.23 mmol). The reaction medium was stirred at 0° C. for 1 hour then brought to room temperature for 48 hours. The dicyclohexylurea precipitate was filtered and washed with dichloromethane and the filtrate was evaporated. The residue obtained (3.68 g) was purified by chromatography on silica gel (eluent: dichloromethane/ethyl acetate 95:5) to give the desired compound in the form of a white powder.

Yield: 96%

Rf: (dichloromethane/methanol 98:2): 0.63

IR: vNH amide 3276 cm⁻¹; vCO ester 1745 cm⁻¹; vCO amide 1649 cm⁻¹

MP: 81.5-82.5° C.

NMR (¹H, CDCl₃): 0.89 (t, 6H, CH₃, J=6.6 Hz); 1.26-1.37 (multiplet, 44H, —CH₂—); 1.56-1.61 (m, 4H, —CH₂—CH₂—S—CH₂—CONH); 2.50-2.60 (m, 4H, —CH₂—CH₂—S—CH₂—CONH—); 3.22 (s, 2H, —CH₂—S—CH₂—CONH—); 3.25 (s, 2H, —CH₂—S—CH₂—CONH—); 3.74 (m, 2H, —OCO—CH₂—CH—CH₂—NHCO—); 3.79 (s, 3H, —COOCH₃); 4.64-4.70 (m, 1H, —OCO—CH₂—CH—CH₂—NHCO—); 7.79 (d, 2H, —NHCO—, J=7.3 Hz).

MS (MALDI-TOF): M+1=659 (M+H⁺); M+23=681 (M+Na⁺); M+39=697 (M+K⁺)

### Preparation of 2 3-ditetradecylthioacetylaminopropan-1-ol (example 18)

Sodium borohydride (316 mg, 8.4 mmol) was dissolved in tetrahydrofuran (40 ml). The reaction mixture was cooled in an ice bath followed by the addition of methyl 2,3-ditetradecylthioacetylaminopropanoate (example 18b) (500 mg, 0.76 mmol) in small portions. The mixture was brought to room temperature and stirred. After 4 days of reaction, 20 ml of water were added. The product, which precipitated, was filtered, washed with water then dried in a dessicator to give a white powder.

Yield: 76%

Rf: (dichloromethane/methanol 95:5): 0.53

IR :vOH alcohol 3436 cm⁻¹; vNH amide 3313 and 3273 cm⁻¹; vCO amide 1648 and 1622 cm⁻¹

MP: 100.2-102.2° C.

NMR (¹H, CDCl₃): 0.89 (t, 6H, CH₃, J=6.2 Hz); 1.26 (multiplet, 44H, —CH₂—); 1.59 (m, 4H, —CH₂—CH₂—S—CH₂—CONH); 2.50-2.56 (m, 4H, —CH₂—CH₂—S—CH₂—CONH—); 3.23 (s, 2H, —CH₂—S—CH₂—CONH—); 3.27 (s, 2H, —CH₂—S—CH₂—CONH—); 3.50-3.91 (multiplet, 5H, —OCO—CH₂—CH—CH₂—NHCO—); 7.38 (d, 2H, —NHCO—, J=7.1 Hz).

MS (MALDI-TOF): M+1=631 (M+H⁺); M+23=653 (M+Na⁺); M+39=669 (M+K⁺)

### Example 19

### Preparation of 2,3-ditetradecylthioacetylamino-1-tetradecylthioacetyloxypropane

2,3-ditetradecylthioacetylaminopropan-1-ol (example 18) (0.200 g, 0.32 mmol) was dissolved in tetrahydrofuran (40 ml) followed by the addition of dicyclohexylcarbodiimide (65 mg, 0.32 mmol), dimethylaminopyridine (39 mg, 0.32 mmol) and tetradecylthioacetic acid (example 1a) (91 mg, 0.32 mmol). The mixture was stirred at room temperature for 20 hours. The dicyclohexylurea precipitate was filtered, washed with tetrahydrofuran and the filtrate was evaporated. The residue obtained (1 g) was purified by flash chromatography (eluent: dichloromethane 10) to produce the desired compound in the form of a white powder.

Yield: 59%

Rf: (dichloromethane/ethyl acetate 8:2): 0.49

IR: vNH amide 3281 cm⁻¹; vCO ester 1736 cm⁻¹; vCO amide 1641 cm⁻¹

MP: 95.4-97.3° C.

NMR (¹H, CDCl₃): 0.89 (t, 9H, CH₃, J=6.4 Hz); 1.27-1.34 (multiplet, 66H, —CH₂—); 1.54-163 (m, 6H, —CH₂—CH₂—S—CH₂—CO—); 2.53 (t, 4H, —CH₂—CH₂—S—CH₂—CONH—, J=7.2 Hz); 2.65 (t, 2H, —CH₂—CH₂—S—CH₂—COO—, J=7.2 Hz); 3.21 (s, 2H, —CH₂—S—CH₂—CONH—); 3.23 (s, 2H, —CH₂—S—CH₂—CONH—); 3.25 (s, 2H, —CH₂—S—CH₂—COO—); 3.46-3.56 (m, 2H, —OCO—CH₂—CH—CH₂—NHCO—); 4.22-4.25 (m, 2H, —OCO—CH₂—CH—CH₂—NHCO—); 4.29-4.39 (m, 2H, —OCO—CH₂—CH—CH₂—NHCO—); 7.29 (t, 1H, —NHCO—); 7.38 (d, 1H, —NHCO—, J=7.6 Hz).

MS (MALDI-TOF): M+1=901 (M+H⁺)

### Example 20

### Preparation of 1,3-diamino-2-(tetradecylthioacetylthio)propane dihydrochloride

### Preparation of 1,3-di(tert-butyloxycarbonylamino)-2-(p-toluenesulfonyloxy) propane (example 20a)

1,3-di(tert-butyloxycarbonylamino)propan-2-ol (example 15a) (2.89 g, 10 mmol) and triethylamine (2.22 ml, 16 mmol) were dissolved in anhydrous dichloromethane (100 ml). The reaction mixture was cooled in an ice bath followed by dropwise addition of tosyl chloride (2.272 g, 12 mmol) dissolved in dichloromethane (30 ml). The reaction mixture was then stirred at room temperature for 72 hours. One equivalent of chloride and 1.6 of triethylamine (TEA) were added after 48 hours. Water was added to stop the reaction and the medium was allowed to settle. The organic phase was washed several times with water. The aqueous phases were combined and extracted again with dichloromethane. The organic phase was dried on magnesium sulfate, filtered and the solvent was evaporated. The residue obtained (6.44 g) was purified by chromatography on silica gel (eluent:dichloromethane followed by dichloromethane/methanol 99:1) to yield the desired compound as a white solid.

Yield: 48%

Rf (dichloromethane/methanol 98:2): 0.70

IR: vNH 3400 cm⁻¹; vCO ester 1716 cm⁻¹; vCO carbamate 1689 cm⁻¹

MP: 104-111° C.

NMR (¹H, CDCl₃): 1.42 (s, 18H, CH₃ (BOC)); 2.46 (s, 3H, CH₃); 3.22 and 3.41 (multiplet, 4H, BOCNH—CH₂—CH—CH₂—NHBOC); 4.56 (m, 1H, BOCNH—CH₂—CH—CH₂—NHBOC); 5.04-5.11 (multiplet, 2H, —NHBOC); 7.36 (d, 2H, aromatics, J=8.5 Hz); 7.36 (d, 2H, aromatics, J=8.5 Hz).

MS (MALDI-TOF): M+23=467 (M+Na⁺); M+39=483 (M+K⁺)

### Preparation of 1,3-di(tert-butyloxycarbonylamino)-2-acetylthiopropane (example 20b)

1,3-(ditert-butoxycarbonylamino)-2-(p-toluenesulfonyloxy)propane (example 20a) (0.500 g, 1.12 mmol) and potassium thioacetate (0.161 g, 1.41 mmol) were dissolved in acetone and the medium was refluxed for 48 hours. One equivalent of potassium thioacetate was added after 24 hours of reflux. The reaction was brought to room temperature and the solvent evaporated. The residue was taken up in diethyl ether and filtered on Celite®. The filtrate was evaporated. The product obtained (0.48 g) was purified by chromatography on silica gel (eluent:dichloromethane/ethyl acetate 98:2) to give the desired compound as an ochre solid.

Yield: 84%

Rf (dichloromethane/methanol 98:2): 0.45

IR: vNH 3350 cm⁻¹; vCO ester 1719 cm⁻¹; vCO carbamate 1691 cm⁻

MP: 93-96° C.

NMR (¹H, CDCl₃): 1.45 (s, 18H, CH₃ (BOC)); 2.34 (s, 3H, CH₃); 3.23-3.32 (m, 2H, BOCNH—CHₐH_{b}—CH—CHₐH_{b}—NHBOC); 3.38-3.43 (m, 2H, BOCNH—CHₐH_{b}—CH—CHₐH_{b}—NHBOC); 3.58-3.66 (m, 1H, BOCNH—CH₂—CH—CH₂—NHBOC); 5.22 (multiplet, 2H, —NHBOC).

MS (MALDI-TOF): M+23=371 (M+Na⁺)

### Preparation of 1,3-di(tert-butyloxycarbonylamino)-2-mercaptopropane (example 20c)

1,3-di(tert-butoxycarbonylamino)-2-(acetylthio)propane (example 20b) (0.380 g, 1.2 mmol) diluted in methanol (10 ml) was added to a 20% potassium carbonate solution in methanol (2.14 ml, 12.4 mmol), degassed under a stream of nitrogen. The reaction mixture was stirred under nitrogen at room temperature for 20 hours, then acidified to pH 6 with acetic acid. The solvents were vacuum evaporated. The residue was taken up in water and extracted with chloroform. The organic phases were combined, dried on magnesium sulfate, then filtered and dried to give the desired product in the form of a white solid which was promptly used in the next reaction.

Yield: 90%

Rf (dichloromethane/methanol 98:2): 0.56

IR: vNH 3370 cm⁻¹; vCO carbamate 1680 cm⁻¹

NMR (¹H, CDCl₃): 1.46 (s, 18H, CH₃ (BOC)); 2.98-3.12 (multiplet, 3H, BOCNH—CHₐH_{b}—CH—CHₐH_{b}—NHBOC and BOCNH—CH₂—CH—CH₂—NHBOC); 3.46-3.50 (m, 2H, BOCNH—CHₐH_{b}—CH—CHₐH_{b}—NHBOC); 5.27 (multiplet, 2H, —NHBOC).

### Preparation of 1,3-di(tert-butyloxycarbonylamino)-2-(tetradecylthioacetylthio)propane (example 20d)

1,3-[di(tert-butoxycarbonylamino)]-2-mercaptopropane (example 20c) (0.295 g, 0.963 mmol) was dissolved in dichloromethane (40 ml). Dicyclohexylcarbodiimide (0.199 g, 0.963 mmol), dimethylaminopyridine (0.118 g, 0.963 mmol) and tetradecylthioacetic acid (example 1a) (0.278 g, 0.963 mmol) were then added. The reaction mixture was stirred at room temperature. After 20 hours of reaction, the dicyclohexylurea precipitate was filtered, washed with dichloromethane and the filtrate was evaporated. The residue obtained (0.73 g) was purified by chromatography on silica gel (eluent:dichloromethane) to give the desired compound in the form of a white powder.

Yield: 72%

Rf (dichloromethane/ethyl acetate 95:5): 0.29

IR: vNH 3328 cm⁻¹; vCO thioester 1717 cm⁻¹; vCO carbamate 1687 cm⁻¹

MP: 47-51° C.

NMR (¹H, CDCl₃): 0.88 (t, 9H, CH₃, J=6.1 Hz); 1.26 (multiplet, 22H, —CH₂—); 1.44 (s, 18H, CH₃ (BOC)); 1.53-1.65 (m, 2H, —CH₂—CH₂—S—CH₂—CO); 2.59 (t, 2H, —CH₂—CH₂—S—CH₂—COS—, J=7.8 Hz); 3.21-3.30 (m, 2H, BOCNH—CHₐH_{b}—CH—CHₐH_{b}—NHBOC); 3.40 (s, 2H, CH₂—S—CH₂—COS—); 3.42-3.49 (m, 2H, BOCNH—CHₐH_{b}—CH—CHₐH_{b}—NHBOC); 3.62-3.65 (m, 1 H, BOCNH—CH₂—CH—CH₂—NHBOC); 5.24 (multiplet, 2H, —NHBOC).

MS (MALDI-TOF): M+23=599 (M+Na⁺); M+39=615 (M+K⁺)

### Preparation of 1,3-diamino-2-(tetradecylthioacetylthio)propane dihydrdochloride (example 20)

1,3-[di(tert-butoxycarbonylamino)]-2-tetradecylthioacetylthiopropane (example 20d) (0.300 g, 0.52 mmol) was dissolved in ether saturated with gaseous hydrochloric acid (55 ml). The mixture was stirred at room temperature. After 96 hours of reaction, the precipitate which formed was filtered, washed several times with diethyl ether and dried to give the desired compound in the form of a dihydrochloride (white powder).

Yield: 59%

Rf (dichloromethane /methanol 9:1): 0.11

IR: vNH.HCl 2700-3250 cm⁻¹; vCO thioester 1701 cm⁻¹

MP: 181° C. (decomposition)

NMR (¹H, CDCl₃): 0.86 (t, 3H, CH₃, J=6 Hz); 1.24 (multiplet, 22H, —CH₂—); 1.49-1.54 (m, 2H, —CH₂—CH₂—S—CH₂—CO); 2.59 (m, 2H, —CH₂—CH₂—S—CH₂—COS—); 2.80-2.84 (m, 1 H, BOCNH—CHₐH_{b}—CH—CHₐH_{b}—NHBOC); 3.03-3.09 (m, 1H, BOCNH—CHₐH_{b}—CH—CHₐH_{b}—NHBOC); 3.14 (s, 2H, CH₂—S—CH₂—COS—); 3.27-3.38 (m, 2H, BOCNH—CHₐH_{b}—CH—CHₐH_{b}—NHBOC); 3.86-3.90 (m, 1H, BOCNH—CH₂—CH—CH₂—NHBOC); 8.21 and 8.52 (2m, 2H+4H, NH₂.HCl).

### Example 21

### Preparation of 1,3-ditetradecylthioacetylamino-2-(tetradecylthioacetylthio)propane

1,3-diamino-2-tetradecylthioacetylthiopropane dihydrochloride (example 20) (100 mg, 0.225 mmol) and tetradecylthioacetic acid (example 1a) (130 mg, 0.45 mmol) were dissolved in dichloromethane (30 ml) at 0° C. followed by the addition of triethylamine (68 μl), dicyclohexylcarbodiimide (139 mg, 0.675 mmol) and hydroxybenzotriazole (61 mg, 0.450 mmol). The reaction mixture was stirred at 0° C. for 1 hour then brought to room temperature for 48 hours. The dicyclohexylurea precipitate was filtered and washed with dichloromethane and the filtrate was evaporated. The residue obtained (430 mg) was purified by chromatography on silica gel (eluent : dichloromethane/ethyl acetate 95:5) to give the desired compound in the form of a white powder.

Yield: 82%

Rf (dichloromethane/methanol 98:2): 0.54

IR: vCO thioester 1660 cm⁻¹; vCO amide 1651 cm⁻¹

MP: 83-85° C.

NMR (¹H, CDCl₃): 0.89 (t, 9H, CH₃, J=6.6 Hz); 1.26 (multiplet, 66H, —CH₂—); 1.56-1.62 (multiplet, 6H, —CH₂—CH₂—S—CH₂—CO); 2.56 (t, 4H, —CH₂—CH₂—S—CH₂—CONH—, J=7.5 Hz); 2.61 (t, 2H, —CH₂—CH₂—S—CH₂—COS—, J=7 Hz); 3.26 (s, 4H, CH₂—S—CH₂—CONH—); 3.42 (s, 2H, CH₂—S—CH₂—COS—); 3.44-3.49 (m, 2H, —CONH—CHₐH_{b}—CH—CHₐH_{b}—NH—CO); 3.55-3.61 (m, 2H, —CONH—CHₐH_{b}—CH—CHₐH_{b}—NHCO—); 3.70-3.71 (m, 1H, BOCNH—CH₂—CH—CH₂—NHBOC); 7.58-7.62 (m, 2H, NHCO).

MS (MALDI-TOF): M+1=917 (M+H⁺); M+23=939 (M+Na⁺)

### Example 22

### Preparation of 1-amino-2,3-di(tetradecylthioacetylthio)propane hydrochloride

Preparation of 1-(tert-butyloxycarbonylamino)propane-2,3-diol (example 22a)

1-aminopropane-2,3-diol (5 g, 55 mmol) was dissolved in methanol (200 ml) followed by dropwise addition of triethylamine (0.5 ml per mmol of amine) and di-tert-butyl dicarbonate [(BOC)2O] (wherein BOC corresponds to tertbutyloxycarbonyl) (17.97 g, 82 mmol). The reaction medium was heated at 40-50° C. for 20 min then stirred at room temperature for 1 hour. After evaporation of the solvent, the colorless oily residue was purified by chromatography on silica gel (eluent : dichloromethane/methanol 95:5) to give the desired compound in the form of a colorless oil which crystallized slowly.

Yield: 99%

Rf (dichloromethane/methanol 9:1): 0.39

IR: vNH 3350 cm⁻¹; vCO ester 1746 cm⁻¹; vCO amide 1682 cm⁻¹

MP<15° C.

NMR (¹H, CDCl₃): 1.44 (s, 9H, CH₃ (BOC)); 3.16-3.31 (m, 2H, BOCNH—CH₂—CH—CH₂—OH); 3.44 (multiplet, 2H, OH); 3.16-3.31 (m, 2H, BOCNH—CH₂—CH—CH₂—OH); 3.71-3.78 (m, 1H, BOCNH—CH₂—CH—CH₂—OH); 5.24 (m, 1H, —NHBOC).

MS (MALDI-TOF): M+23=214 (M+Na⁺)

### Preparation of 1-(tert-butyloxycarbonylamino)-2,3-di(p-toluenesulfonyloxy)propane (example 22b)

This compound was synthesized according to the method described hereinabove (example 20a) from 1-(tert-butyloxycarbonylamino)-propane-2,3-diol (example 22a) and p-toluenesulfonyl chloride. The reaction produced a white powder.

Yield: 45%

Rf (dichloromethane/methanol 98:2): 0.49

IR: vNH 3430 cm⁻¹; vCO ester and carbamate 1709 cm⁻¹

MP: 112-116° C.

NMR (¹H, CDCl₃): 1.40 (s, 9H, CH₃ (BOC)); 2.46 (s, 6H, CH₃); 3.26-3.45 (m, 2H, BOCNH—CH₂—CH—CH₂—OTs); 4.04-4.14 (m, 2H, BOCNH—CH₂—CH—CH₂—OTs); 4.68 (m, 1H, BOCNH—CH₂—CH—CH₂—OTs); 4.71 (s, 1H, —NHBOC); 7.34 (d, 4H, aromatics, J=8.5 Hz); 7.69 (d, 2H, aromatics, J=8.1 Hz); 7.76 (d, 2H, aromatics, J=8.1 Hz).

MS (MALDI-TOF): M+23=522 (M+Na⁺); M+39=538 (M+K⁺)

### Preparation of 1-(tert-butyloxycarbonylamino)-2,3-di(acetylthio)propane (example 22c)

This compound was synthesized according to the method described hereinabove (example 20b) from 1-(tert-butyloxycarbonylamino)-2,3-di(p-toluenesulfonyloxy)propane (example 22b) and potassium thioacetate. The reaction produced a white solid.

Yield: 59%

Rf (dichloromethane /ethyl acetate 95:5): 0.55

IR: vNH 3430 cm⁻¹; vCO thioester 1718 cm⁻¹; vCO carbamate 1690 cm⁻¹

MP: 62-63° C.

NMR (¹H, CDCl₃): 1.45 (s, 9H, CH₃ (BOC)); 2.35 (s, 3H, CH₃); 2.37 (s, 3H, CH₃); 3.12-3.38 (multiplet, 4H, BOCNH—CH₂—CH—CH₂—SCO—); 3.69-3.78 (m, 1H, BOCNH—CH₂—CH—CH₂—SCO—); 5.02 (s, 1 H, —NHBOC).

MS (MALDI-TOF): M+23=330 (M+Na⁺)

### Preparation of 1-(tert-butyloxycarbonylamino)-2,3-dimercaptopropane (example 22d)

This compound was synthesized according to the method described hereinabove (example 20c) by saponification of 1-(tert-butyloxycarbonylamino)-2,3-di(acetylthio)-propane (example 22c). The reaction produced a white solid which was promptly used in the next reaction.

Yield: 95%

Rf (dichloromethane/ethyl acetate 95:5): 0.45

IR: vNH 3368 cm⁻¹; vCO carbamate 1688 cm⁻¹

MP: 62-63° C.

NMR (¹H, CDCl₃): 1.46 (s, 9H, CH₃ (BOC)); 3.04-3.11 (m, 1H, BOCNH—CH₂—CHSH—CH₂—SH); 3.26-3.35 (m, 2H, BOCNH—CH₂—CHSH—CH₂—SH); 3.43-3.52 (m, 2H, BOCNH—CH₂—CH—CH₂—SH); 4.91 (m, 2H, SH); 5.08 (s, 1 H, —NHBOC).

### Preparation of 1-(tert-butyloxycarbonylamino)-2,3-di(tetradecylthioacetylthio)propane (example 22e)

This compound was synthesized according to the method described hereinabove (example 20d) from 1-(tert-butyloxycarbonylamino)-2,3-dimercaptopropane (example 22d) and tetradecylthioacetic acid (example 1a). The reaction produced a white solid.

Yield: 50%

Rf (dichloromethane): 0.38

IR: vNH 3421 cm⁻¹; vCO thioester 1721 cm⁻¹; vCO carbamate 1683 cm⁻¹

MP: 60-62° C.

NMR (¹H, CDCl₃): 0.87 (t, 6H, CH₃, J=6.3 Hz); 1.26 (multiplet, 44H, —CH₂—); 1.45 (s, 9H, CH₃ (BOC)); 1.57-1.62 (m, 4H, —CH₂—CH₂—S—CH₂—COS—); 2.60 (t, 4H, —CH₂—CH₂—S—CH₂—COS—, J=6.9 Hz); 3.17-3.29 (m, 2H, BOCNH—CHₐH_{b}—CH—CHₐH_{b}—NHBOC); 3.29-3.38 (m, 2H, BOCNH—CHₐH_{b}—CH—CHₐH_{b}—NHBOC); 3.41 (s, 2H, CH₂—S—CH₂—COS—); 3.43 (s, 2H, CH₂—S—CH₂—COS—); 3.76-3.80 (m, 1H, BOCNH—CH₂—CH—CH₂—NHBOC); 5.03 (s,1 H, —NHBOC).

MS (MALDI-TOF): M+23=786 (M+Na⁺)

### Preparation of 1-amino-2.3-di(tetradecylthioacetylthio)propane hydrochloride (example 22)

This compound was synthesized according to the method described hereinabove (example 20) from 1-(tert-butyloxycarbonylamino)-2,3-ditetradecylthioacetylthiopropane (example 22e). The reaction produced the hydrochloride (white solid).

Yield: 43%

Rf (dichloromethane): 0.19

IR: vNH.HCl 2700-3250 cm⁻¹; vCO thioester 1701 and 1676 cm⁻¹

MP:117-128° C.

NMR (¹H, CDCl₃): 0.86 (t, 6H, CH₃, J=6 Hz); 1.24 (multiplet, 44H, —CH₂); 1.51 (m, 4H, —CH₂—CH₂—S—CH₂—COS—); 2.61 (m, 4H, —CH₂—CH₂—S—CH₂—COS—); 2.93-3.04 (m, 2H, —S—CHₐH_{b}—CH—CHₐH_{b}—NH₂.HCl); 3.11-3.20 (m, 2H, —S—CHₐH_{b}—CH—CHₐH_{b}—NH₂.HCl); 3.59-3.63 (multiplet, 4H, CH₂—S—CH₂—COS—); 3.72-3.84 (m,1 H, —S—CH₂—CH—CH₂—NH₂.HCl); 8.12 (m, 3H, NH₂.HCl).

### Example 23

### Preparation of 1-tetradecylthioacetylamino-2,3-di(tetradecylthioacetylthio)propane

1-amino-2,3-ditetradecylthioacetylthiopropane hydrochloride (example 22) (100 mg, 0.140 mmol) and tetradecylthioacetic acid (example 1a) (62 mg, 0.210 mmol) were dissolved in dichloromethane (40 ml) at 0° C. followed by the addition of triethylamine (43 ml), dicyclohexylcarbodiimide (59 mg, 0.28 mmol) and hydroxybenzotriazole (29 mg, 0.210 mmol). The reaction mixture was stirred at 0° C. for 1 hour then brought to room temperature for 24 hours. It was then heated under gentle reflux for 48 hours and dried. The residue obtained (310 mg) was purified by chromatography on silica gel (eluent dichloromethane/cyclohexane 8:2) and produced the desired comopund as a white powder.

Yield: 96%

Rf (dichloromethane): 0.20

IR: vNH amide 3306 cm⁻¹; vCO thioester 1674 cm⁻¹; vCO amide 1648 cm⁻¹

MP: 78-80° C.

NMR (¹H, CDCl₃): 0.89 (t, 9H, CH₃, J=6.6 Hz); 1.26 (multiplet, 66H, —CH₂); 1.58-1.62 (multiplet, 6H, —CH₂—CH₂—S—CH₂—COS—); 2.56 (t, 4H, —CH₂—CH₂—S—CH₂—COS—, J=7.5 Hz); 2.61 (t, 2H, —CH₂—CH₂—S—CH₂—CONH—J=7 Hz); 3.26 (s, 4H, CH₂—S—CH₂—COS—); 3.42 (s, 2H, CH₂—S—CH₂—CONH—); 3.44-3.49 (m, 2H, BOCNH—CHₐH_{b}—CH—CHₐH_{b}—NHCO—); 3.55-3.61 (m, 2H, —S—CHₐH_{b}—CH—CHₐH_{b}—NHCO—); 3.70-3.71 (m, 1H, —S—CH₂—CH—CH₂—NHCO—); 7.58-7.62 (m, 1H, NHCO).

MS (MALDI-TOF): M+1=934 (M+H⁺); M+23=956 (M+Na⁺); M+39=972 (M+K⁺)

### Example 24

### Preparation of 1-tetradecylthioacetylthio-2.3-di(tetradecylthioacetylamino)propane

### Preparation of 2,3-di(tetradecylthioacetylamino)-1-iodopropane (example 24a)

2,3-ditetradecylthioacetylaminopropan-1-ol (example 18) (0.200 g, 0.317 mmol) was dissolved in toluene (30 ml). Imidazole (0.054 g, 0.792 mmol), triphenylphosphine (0.208 g, 0.792 mmol) and iodine (0.161 g, 0.634 mmol) were then added in that order and the reaction was heated at 75-80° C. with stirring. After 6 hours of reaction, the solvent was evaporated and the residual product was used without further purification.

Rf (dichloromethane/methanol 98:2): 0.55

### Preparation of 2,3-di(tetradecylthioacetylamino)-1-mercaptopropane (example 24b)

Sodium hydrogen sulfide (0.089 g, 1.59 mmol) was added to 2,3-ditetradecylthioacetylamino-1-iodopropane (example 24a) (0.235 g, 0.32 mmol) dissolved in acetone (80 ml). The reaction medium was heated at 70° C. for 16 hours. The solvent was evaporated and the residue taken up in water and extracted with chloroform. The aqueous phase was acidified to pH 6 with acetic acid, then extracted again with chloroform. The organic phases were dried on magnesium sulfate and filtered and the solvent was evaporated. The residue obtained was used without further purification.

### Preparation of 1-tetradecylthioacetylthio-2,3-di(tetradecylthioacetylamino)propane (example 24)

2,3-ditetradecylthioacetylamino-1-mercaptopropane (example 24b) (0.205 g, 0.32 mmol) was dissolved in tetrahydrofuran (50 ml). Dicyclohexylcarbodiimide (98 mg, 0.47 mmol), dimethylaminopyridine (58 mg, 0.47 mmol) and tetradecylthioacetic acid (example 1a) (137 mg, 0.47 mmol) were then added. The mixture was stirred at room temperature for 20 hours. The dicyclohexylurea precipitate was filtered, washed with tetrahydrofuran and the filtrate was evaporated. The residue obtained (1.14 g) was purified by chromatography on silica gel (eluent:dichloromethane) to give the desired compound in the form of an ochre powder.

Yield: 10%

Rf (dichloromethane/ethyl acetate 98:2): 0.19

IR: vCO thioester 1711-1745 cm⁻¹; vCO amide 1651 cm⁻¹

MP: 48.8-49.8° C.

NMR (¹H, CDCl₃): 0.89 (t, 9H, CH₃, J=6.3Hz); 1.26 (multiplet, 66H, —CH₂); 1.58 (m, 6H, —CH₂—CH₂—S—CH₂—COS—); 2.46-55 (m, 4H, —CH₂—CH₂—S—CH₂—CONH); 2.65 (t, 2H, —CH₂—CH₂—S—CH₂—COS—, J=7.4 Hz); 3.24 (s, 2H, CH₂—S—CH₂—CONH—); 3.26 (s, 2H, CH₂—S—CH₂—CONH—); 3.66 (t, 2H, —COS—CH₂—CH—CH₂—NHCO); 3.79 (t, 2H, CH₂—S—CH₂—COS—, J=6.3 Hz); 4.31-4.41 (m, 2H, —COS—CH₂—CH—CH₂—NHCO); 5.00-5.05 (m, 1 H, —COS—CH₂—CH—CH₂—NHCO); 7.33 (sl, 1 H, NHCO); 9.27 (d, 1H, NHCO, J=8.6 Hz).

MS (MALDI-TOF): M+1=917 (M+H⁺); M+23=939 (M+Na⁺); M+39=955 (M+K⁺)

### Example 25

### Preparation of 3-tetradecylthioacetylamino-2-tetradecylthioacetylthiopropan-1-ol

### Preparation of 3-tetradecylthioacetylamino- 1-triphenylmethyloxypropan-2-ol (example 25a)

Chlorotriphenylmethane (2.833 g, 10.16 mmol) was added to a solution of 3-tetradecylthioacetylaminopropane-1,2-diol (example 7) (3 g, 8.3 mmol) in pyridine (2.5 ml). The reaction mixture was stirred at 50° C. for 24 hours and the solvent was then vacuum evaporated. The residue was taken up in water and extracted with dichloromethane. The organic phase was washed with 1N aqueous hydrochloric acid then with an aqueous saturated sodium chloride solution. It was dried on magnesium sulfate, filtered and the solvent was evaporated. The residue obtained (6.36 g) was purified by chromatography on silica gel (eluent:dichloromethane/ethyl acetate 98:2) to give the desired compound in the form of a white powder.

Yield: 69%

Rf (dichloromethane/ethyl acetate 8:2): 0.61

IR: vNH amide 3225 cm⁻¹; vCO amidel654 cm⁻¹

MP: 62.6-65.4° C.

NMR (¹H, CDCl₃): 0.89 (t, 3H, CH₃, J=6.7 Hz); 1.26 (multiplet, 22H, —CH₂); 1.50-1.57 (m, 2H, —CH₂—CH₂—S—CH₂—CONH—); 2.48 (t, 2H, —CH₂—CH₂—S—CH₂—CONH, J=7.2 Hz); 3.01 (m, 1H, OH); 3.17 (s, 2H, CH₂—S—CH₂—CONH—); 3.19 (m, 2H, —O—CH₂—CH—CH₂—NHCO or trityl-O—CH₂—CH—CH₂—NHCO); 3.27-3.36 (m, 1H, —O—CH₂—CH—CH₂—NHCO or trityl-O—CH₂—CH—CH₂—NHCO); 3.54-3.62 (m, 1 H, —O—CH₂—CH—CH₂—NHCO or trityl-O—CH₂—CH—CH₂—NHCO); 3.93 (m, 1 H, —O—CH₂—CH—CH₂—NHCO); 7.16 (t, 1H, NHCO, J=5.7 Hz); 7.23-7.35 (multiplet, 9H, aromatic H); 7.41-7.45 (multiplet, 6H, aromatic H).

MS (MALDI-TOF): M+23=626 (M+Na⁺)

### Preparation of 2-iodo-3-tetradecylthioacetylamino-1-triphenylmethyloxypropane (example 25b)

3-tetradecylthioacetylamino-1-triphenylmethyloxypropan-2-ol (example 25a) (2 g, 3.31 mmol) was dissolved in toluene (100 ml). Imidazole (0.564 g, 8.28 mmol), triphenylphosphine (2.171 g, 8.28 mmol) and iodine (1.681 g, 6.62 mmol) were then added in that order. The reaction medium was stirred at room temperature for 20 hours. A saturated sodium bisulfite solution was added until complete blanching of the reaction medium. The phases were separated and the aqueous phase was extracted with toluene. The organic phases were combined, washed with saturated sodium chloride solution, dried on magnesium sulfate and filtered. The residue obtained after evaporation of the solvent (4.65 g) was purified by chromatography on silica gel (eluent:dichlromethane) to give the desired compound in the form of a yellow oil.

Yield: 21%

RF (Dichloromethane/Ethyl Acetate 95:5): 0.58

IR: vCO amide 1668 cm⁻¹; vCH arom. monosubstituted 748 and 698 cm⁻¹

NMR (¹H, CDCl₃) : 0.89 (t, 3H, CH₃, J=6.5 Hz); 1.26 (multiplet, 20H, —CH₂); 1.53-1.63 (m, 2H, —CH₂—CH₂—CH₂—S—CH₂—CONH—); 2.63 (m, 2H, —CH₂—CH₂—CH₂—S—CH₂—CONH); 3.13-3.30 (m, 2H, —CH₂—S—CH₂—CONH); 3.34 (s, 2H, CH₂—S—CH₂—CONH); 3.67-3.71 (m, 2H, —O—CH₂—CH—CH₂—NHCO or trityl-O—CH₂—CH—CH₂—NHCO); 3.88-3.94 (m, 2H, —O—CH₂—CH—CH₂—NHCO or trityl-O—CH₂—CH—CH₂—NHCO); 4.76 (m, 1H, —O—CH₂—CH—CH₂—NHCO); 7.25-7.36 (multiplet, 9H, aromatic H); 7.45-7.49 (multiplet, 6H, aromatic H).

MS (MALDI-TOF): M−127=586 (M−I)

### Preparation of 2-mercapto-3-tetradecylthioacetylamino-1-triphenylmethyloxypropane (example 25c)

Sodium hydrogen sulfate hydrate (38 mg, 0.68 mmol) was prepared as a suspension in ethanol (20 ml) followed by the addition of 2-iodo-3-tetradecylthioacetylamino-1-triphenylmethyloxypropane (example 25b) (200 mg, 0.28 mmol). The reaction medium was heated at 70° C. 238 mg of sodium hydrogen sulfate hydrate were added over several days. After 6.5 days, the solvent was evaporated and the residue taken up in dichloromethane and washed with water. The aqueous phase was re-extracted and the combined organic phases were washed with 0.5N hydrochloric acid then with saturated sodium chloride solution, then dried on magnesium sulfate. The salt was filtered and the solvent evaporated. The residue obtained was used without further purification.

Rf (dichloromethane/ethyl acetate 95:5): 0.33

### Preparation of 3-tetradecylthioacetylamino-2-tetradecylthioacetylthio-1-triphenylmethyloxy-propane (example 25d)

2-mercapto-3-tetradecylthioacetylamino-1-triphenylmethyloxypropane (example 25c) (174 mg, 0.28 mmol) was dissolved in tetrahydrofuran (20 ml). Dicyclohexylcarbodiimide (88 mg, 0.42 mmol), dimethylaminopyridine (51 mg, 0.42 mmol) and tetradecylthioacetic acid (example 1a) (121 mg, 0.42 mmol) were then added and the reaction medium was stirred at room temperature. After 20 hours of reaction, the solvent was evaporated and the residue obtained (450 mg) was purified by flash chromatography (eluent:dichloromethane/cyclohexane 3:7 to 5-5) to give the desired compound in the form of a white powder.

Yield :76%

Rf (dichloromethane): 0.39

IR: vCO thioester and amide 1745 to 1640 cm⁻¹

MP: 48.5-51.9° C.

NMR (¹H, CDCl₃) : 0.89 (t, 6H, CH₃, J=6.3 Hz); 1.26 (multiplet, 44H, —CH₂); 1.62 (m, 4H, —CH₂—CH₂—S—CH₂—CO—); 2.42 (t, 2H, —CH₂—CH₂—S—CH₂—CONH—, J=7.5 Hz); 2.68 (t, 2H, —CH₂—CH₂—S—CH₂—COS—, J=7.5 Hz); 3.14 (s, 2H, CH₂—S—CH₂—CONH—); 3.25 (s, 2H, CH₂—S—CH₂—COS—); 3.50-3.59 (m, 1H, —O—CH₂—CH—CH₂—NHCO or trityl-O—CH₂—CH—CH₂—NHCO); 3.66-3.72 (m, 2H, —O—CH₂—CH—CH₂—NHCO or trityl-O—CH₂—CH—CH₂—NHCO); 3.96 (m, 1H, —O—CH₂—CH—CH₂—NHCO or trityl-O—CH₂—CH—CH₂—NHCO); 3.54-3.62 (m, 1H, —O—CH₂—CH—CH₂—NHCO or trityl-O—CH₂—CH—CH₂—NHCO); 5.16 (m, 1H, —O—CH₂—CH—CH₂—NHCO); 7.04 (m, 1H, NHCO, J=5.7Hz); 7.25-7.34 (multiplet, 9H, aromatic H); 7.42-7.45 (multiplet, 9H, aromatic H).

MS (MALDI-TOF): M+23=889 (M+Na⁺)

### Preparation of 3-tetradecylthioacetylamino-2-tetradecylthioacetylthiopropan-1-ol (example 25)

3-tetradecylthioacetylamino-2-tetradecylthioacetylthio-1-triphenylmethyloxypropane (example 25d) (187 mg, 0.21 mmol) was dissolved in ether saturated with gaseous hydrochloric acid (12 ml). The reaction medium was stirred at room temperature for 20 hours. The precipitate which formed was filtered and washed with diethyl ether to give the desired compound in the form of a white powder.

Yield: 52%

Rf (dichloromethane/methanol 98:2): 0.48

IR: vCO thioester 1704cm⁻¹; vCO amide 1646 cm⁻¹

MP: 88.4-94.1° C.

NMR (¹H, CDCl₃): 0.89 (t, 6H, CH₃, J=6.4 Hz); 1.26-1.37 (multiplet, 44H, —CH₂); 1.55-1.61 (m, 4H, —CH₂—CH₂—S—CH₂—CO—); 2.55 (t, 2H, —CH₂—CH₂—S—CH₂—CONH—, J=7 Hz); 2.65 (t, 2H, —CH₂—CH₂—S—CH₂—COS—, J=7 Hz); 3.26 (s, 2H, CH₂—S—CH₂—CONH—); 3.27 (s, 2H, CH₂—S—CH₂—COS—); 3.36-3.38 (m, 1 H, —O—CH₂—CH—CH₂—NHCO); 3.58-3.64 (m, 1 H, —O—CH₂—CH—CH₂—NHCO); 4.02 (m, 1 H, —O—CH₂—CH—CH₂—NHCO); 4.11-4.25 (m, 2H, HO—CH₂—CH—CH₂—NHCO); 7.34 (m,1 H, NHCO).

MS (MALDI-TOF); M+23=670 (M+Na⁺)

### Example 26

### Preparation of 3-tetradecylthioacetylamino-1-tetradecylthiacetyloxy-2-tetradecylthioacetylthiopropane

3-tetradecylthioacetylamino-2-tetradecylthioacetylthiopropan-1-ol (example 25) (64 mg, 0.10 mmol) was dissolved in tetrahydrofuran (7 ml). Dicyclohexylcarbodiimide (31 mg, 0.15 mmol), dimethylaminopyridine (18 mg, 0.15 mmol) and tetradecylthioacetic acid (example 1a) (43 mg, 0.15 mmol) were then added. The mixture was stirred at room temperature for 20 hours. The dicyclohexylurea precipitate was filtered and the filtrate was evaporated. The residue obtained (140 mg) was purified by flash chromatography (eluent:dichloromethane) to give the desired compound in the form of a white powder.

Yield: 17%

Rf ( dichloromethane/ethyl acetate 98:2): 0.23

IR: vCO ester 1730 cm⁻¹; vCO thioester 1671 cm⁻¹; vCO amide 1645 cm⁻¹

MP: 59.0-63.4° C.

NMR (¹H, CDCl₃): 0.89 (t, 9H, CH₃, J=6.5 Hz); 1.26-1.37 (multiplet, 66H, —CH₂); 1.58-1.63 (m, 6H, —CH₂—CH₂—S—CH₂—CO—); 2.53 (t, 2H, —CH₂—CH₂—S—CH₂—CONH—, J=7.6 Hz); 2.61-2.67 (m, 4H, —CH₂—CH₂—S—CH₂—COS— and —CH₂—CH₂—S—CH₂—COO); 3.23 (s, 4H, CH₂—S—CH₂—CONH— and CH₂—S—CH₂—COO—); 3.24 (s, 2H, CH₂—S—CH₂—COS—); 3.50-3.57 (m, 1H, —O—CH₂—CH—CH₂—NHCO); 3.63-3.72 (m, 1H, —O—CH₂—CH—CH₂—NHCO—); 4.19-4.25 (m, 1H, —O—CH₂—CH—CH₂—OCO—); 3.63-3.72 (m, 1H, —O—CH₂—CH—CH₂—OCO—); 5.19 (m, 1H, —O—CH₂—CH—CH₂—NHCO—); 7.20 (m, 1H, NHCO).

MS (MALDI-TOF): M+23=940 (M+Na⁺)

### Example 27

### Preparation of 1-amino-2-tetradecylthioacetyloxy-3-tetradecylthioacetylthiopropane hydrochloride

### Preparation of 1-tert-butyloxycarbonylamino-3-iodopropan-2-ol (example 27a)

1-[(tert-butyloxycarbonyl)amino]propane-2,3-diol (example 22a) (3.88 g, 20 mmol) was dissolved in toluene (250 ml). Imidazole (1.73 g, 25 mmol), triphenylphosphine (6.65 g, 25 mmol) and iodine (5.15 g, 20 mmol) were then added in that order. The reaction medium was stirred at room temperature for 17 hours and 0.5 equivalents of imidazole, triphenylphosphine and iodine were added. After 21 hours of reaction, a saturated sodium sulfite solution was added until complete blanching of the reaction medium. The phases were allowed to settle and the aqueous phase was extracted twice with toluene. The combined organic phases were washed with saturated sodium chloride solution, dried on magnesium sulfate, filtered and the solvent evaporated. The residue obtained (11.02 g) was purified by chromatography on silica gel (eluent dichloromethane/ethyl acetate 95:5) to give the desired compound as a yellow paste which was promptly used in the next reaction.

Yield: 41%

Rf (dichloromethane/methanol 98:2): 0.24

IR: vNH amide 3387 cm⁻¹; vCO carbamate 1678 cm⁻¹

### Preparation of 3-acetylthio-1-tert-butyloxvcarbonylaminopropan-2-ol (example 27b

1-(tert-butyloxycarbonylamino)-3-iodopropan-2-ol (example 27a) (2 g, 6.64 mmol) and potassium thioacetate (0.948 g, 8.30 mmol) were dissolved in acetone (30 ml) and the medium was refluxed for 16 hours. The solvent was vacuum evaporated and the residue was taken up in diethyl ether, then filtered on Celite®. The filtrate was evaporated. The residue obtained (1.69 g) was purified by chromatography on silica gel (eluent:dichloromethane/ethyl acetate 98:2) then repurified by flash chromatography (eluent: dichloromethane) to give the desired compound in the form of a yellow oil.

Yield: 27%

Rf (dichloromethane/ethyl acetate 95:5 ): 0.31

IR: vNH amide 3367 cm⁻¹; vCO thioester 1744 cm⁻¹; vCO carbamate 1697 cm⁻¹

NMR (¹H, CDCl₃): 1.26 (m, 9H, CH₃ (BOC)); 2.37 (s, 3H, COCH₃); 3.04 (m,1 H, —NH—CH₂—CH—CH₂—S— or —NHCH₂—CH—CH₂—S—); 3.24 (m, 1 H, —NH—CH₂—CH—CH₂—S—or —NHCH₂—CH—CH₂—S—); 3.30-3.41 (m, 2H, —NH—CH₂—CH—CH₂—S— or —NHCH₂—CH—CH₂—S—); 4.86 (sl, 1H, OH); 4.96 (m, 1H, —NH—CH₂—CH—CH₂—S—).

### Preparation of 1-tert-butyloxycarbonylamino-3-mercaptopropan-2-ol (example 27c)

3-acetylthio-1-tert-butyloxycarbonylaminopropan-2-ol (example 27b) (0.307 g, 1.23 mmol) diluted in a minimum of methanol (7 ml) was added to a 20% potassium carbonate solution (3.49 ml, 12.31 mmol) in methanol, degassed under a stream of nitrogen. The medium was stirred at room temperature under a stream of nitrogen for 20 hours, then acidified to pH 6 with acetic acid and concentrated to dryness. The residue obtained was taken up in water and extracted with dichloromethane. The organic phase was dried on magnesium sulfate, filtered and concentrated. The oily residue obtained was used immediately in the next reaction without further purification.

Yield: 78%

Rf (dichloromethane/ethyl acetate): 0.07

Preparation of 1-tert-butyloxycarbonylamino-2-tetradecylthioacetyloxy-3-tetradecylthioacetylthiopropane (example 27d)

1-(tert-butyloxycarbonylamino)-3-mercaptopropan-2-ol (example 27c) (0.200 g, 96 mmol) was-dissolved in dichloromethane (50 ml). Dicyclohexylcarbodiimide (0.398 g, 1.93 mmol), dimethylaminopyridine (0.236 g, 1.93 mmol) and tetradecylthioacetic acid (example 1a) (0.557 g, 1.93 mmol) were then added. The mixture was stirred at room temperature for 20 hours. The dicyclohexylurea precipitate was filtered, washed with dichloromethane and the filtrate was evaporated. The residue obtained (1.2 g) was purified by chromatography on silica gel (eluent: dichloromethane) to give the desired compound in the form of a white paste.

Yield: 47%

Rf (dichloromethane): 0.26

IR: vNH amide 3314 cm⁻¹; vCO ester, amide and thioester 1682 to 1744 cm⁻¹

NMR (¹H, CDCl₃): 0.89 (t, 6H, CH₃, J=6.5 Hz); 1.27 (multiplet, 40H, CH₂); 1.45 (multiplet, 9H, CH₃ (BOC)); 1.56-1.63 (m, 4H, —CH₂—CH₂—CH₂—S—CH₂—CO—); 2.65 (m, 4H, —CH₂—CH₂—S—CH₂—CO—); 2.92 (s, 4H, —CH₂—S—CH₂—CO—); 2.96 (m, 4H, —CH₂—S—CH₂—CO—); 3.24-3.40 (m, 2H, —NH—CH₂—CH—CH₂—S— or —NHCH₂—CH—CH₂—S); 3.44-3.51 (m, 2H, —NH—CH₂—CH—CH₂—S— or —NHCH₂—CH—CH₂—S—); 4.91 (m, 1H, —NH—CH₂—CH—CH₂—S—); 5.19 (m, 1H, NHCO).

MS (MALDI-TOF): M+23=770 (M+Na⁺)

### Preparation of 1-amino-2-tetradecylthioacetyloxy-3-tetradecylthioacetylthiopropane hydrochloride (example 27)

1-(tert-butoxycarbonylamino)-2-tetradecylthioacetyloxy-3-tetradecylthioacetylthiopropane (example 27d) (300 mg, 0.40 mmol) was dissolved in diethyl ether saturated with gaseous hydrochloric acid (70 ml) and the reaction medium was stirred at room temperature for 72 hours. The precipitate which formed was filtered, washed with diethyl ether and dried to give the desired compound in the form of a white powder.

Yield: 42%

Rf (dichloromethane/ethyl acetate 90:10): 0

IR: vCO ester 1733 cm⁻¹; vCO thioester 1692 cm⁻¹

MP: 82° C. (decomposition)

NMR (¹H, CDCl₃): 0.86 (t, 6H, CH₃, J=6.6 Hz); 1.24 (multiplet, 44H, —CH₂); 1.52 (m, 4H, —CH₂—CH₂—S—CH₂—CO—); 2.52-2.62 (m, 4H, —CH₂—CH₂—S—CH₂—CO—); 3.07-3.15 (multiplet, 4H, —S—CH₂—CH—CH₂—NH₂); 3.40 (s, 2H, CH₂—S—CH₂—COO—); 3.61 (s, 2H, CH₂—S—CH₂—COS—); 5.12 (m, 1H, —S—CH₂—CH—CH₂—NH₂); 8.01 (m, 3H, —NH₂.HCl).

### Example 28

### Preparation of 1-tetradecylthioacetylamino-2-tetradecylthiacetyloxy-3-tetradecylthioacetylthiopropane

3-amino-2-tetradecylthioacetyloxy-1-tetradecylthioacetyl-thiopropane hydrochloride (example 27) (100 mg, 0.15 mmol) and tetradecylthioacetic acid (example 1a) (63 mg, 0.22 mmol) were dissolved in dichloromethane (30 ml) at 0° C. followed by the addition of triethylamine (0.044 ml), dicyclohexylcarbodiimide (60 mg, 0.29 mmol) and hydroxybenzotriazole (30 mg, 0.22 mmol). The reaction medium was stirred at 0° C. for 1 hour then brought to room temperature for 48 hours. The dicyclohexylurea precipitate was filtered, washed with dichloromethane and the filtrate was evaporated. The residue obtained (263 mg) was purified by flash chromatography (eluent:dichloromethane/ethyl acetate 98:2) to give the desired compound in the form of a white powder.

Yield: 98%

Rf (dichloromethane/ethyl acetate 95:5): 0.38

IR: vNH amide 3340 cm⁻¹; vCO ester 1727 cm⁻¹; vCO amide and thioester 1655 and 1669 cm⁻¹

MP: 63.9-67.1° C.

NMR (¹H, CDCl₃): 0.89 (t, 9H, CH₃, J=6.2 Hz); 1.26 (multiplet, 66H, —CH₂); 1.54-1.66 (m, 6H, —CH₂—CH₂—S—CH₂—CO—); 2.52-2.67 (m, 6H, —CH₂—CH₂—S—CH₂—CO—) 3.08 (m, 1H, —S—CH₂—CH—CH₂—NHCO or —S—CH₂—CH—CH₂—NHCO); 3.21 (s, 2H, CH₂—S—CH₂—CONH—); 3.23 (s, 2H, CH₂—S—CH₂—COO—); 3.27 (m, 1H, —S—CH₂—CH—CH₂—NHCO or —S—CH₂—CH—CH₂—NHCO); 3.43 (s, 2H, CH₂—S—CH₂—COS—); 3.50 (m, 1H, —S—CH₂—CH—CH₂—NHCO or —S—CH₂—CH—CH₂—NHCO); 3.62 (m, 1H, —S—CH₂—CH—CH₂—NHCO or —S—CH₂—CH—CH₂—NHCO); 5.06 (m, 1 H, —COS—CH₂—CH—CH₂—NHCO); 7.24 (t, 1H, —NHCO, J=6.7 Hz).

MS (MALDI-TOF): M+1=918 (M+H⁺); M+23=940 (M+Na⁺)

### Example 29

### Methods of Preparation of the Inventive Compounds for In Vivo Studies and Search for the Most Efficient Preparation

A—Preparation of the Compounds with Carboxymethylcellulose

The carboxymethylcellulose (CMC) which was used is a sodium salt of intermediate viscosity carboxymethylcellulose (Ref. C4888, Sigma-Aldrich, France). The Tween which was used is Polyoxyethylenesorbitan Monooleate (Tween 80, Ref. P8074, Sigma-Aldrich, France).

A 0.5% (mN) solution of CMC was prepared in water and mixed with 0.1% (VN) Tween 80, then stirred overnight. The inventive compounds were then added and dissolved by stirring and ultrasonication for 30 minutes at 60° C.

B—Preparation of the Compounds in Different Surfactants (Cremophor® RH40 and Solutol® HS15)

The emulsion comprising an inventive compound and a surfactant, Cremophor® RH40 (Polyoxyl 40 Hydrogenated Castor Oil) or Solutol® HS15 (polyethylene glycol 660 12-hydroxystearate) was prepared as follows: the inventive compound was dissolved in a solution of Cremophor® RH40 or Solutol® HS15 previously heated in a water-bath at 60° C. in a ratio for example of 6:1 (m/m). The mixture was placed in a water-bath at 60° C. until a homogeneous mixture was obtained. Said mixture was then dispersed by ultrasonication for 20 minutes at 60° C., at which time the solution became translucid. While stirring, water (MilliQ) preheated at 60° C. was added to the solution to give the desired concentration of the compound. The solution was then mixed on a Vortex® mixer, returned to the water-bath (60° C.) and optionally dispersed by ultrasonication for 30 minutes.

Crémophor® RH40 and Solutol® HS15 were from BASF (Ludwigshasen, Germany).

C—Search for the Most Efficient Preparation

The inventors showed that the efficacy of the inventive compounds was better when they were administered in solution with a surfactant.

To this end, the compounds were administered by gavage to Sprague Dawley rats every day for 15 days. Plasma lipids (total cholesterol and triglycerides) were assayed in blood sampled 4 days before administration of inventive compound Ex 4a (D−4), 8 days after (D+8) and 15 days after (D+15) by respectively using the calorimetric assay kits “Cholesterol RTU” and “Enzymatic Triglycerides PAP1000” as directed by the supplier (Bio-Merieux, Marcy l'Etoile, France).

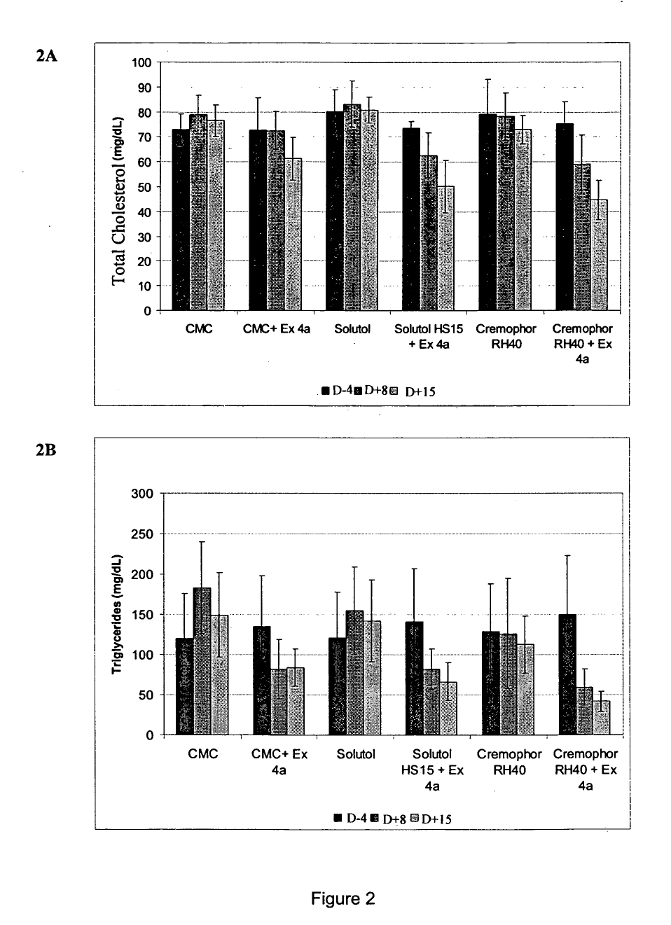

The results (FIG. 2) show that inventive compound Ex 4a induced a larger decrease in total plasma cholesterol (FIG. 2A) and triglycerides (FIG. 2B) when it was administered with Cremophor® RH40.

To carry out the in vivo experiments described in the following examples, the inventive compounds were therefore prepared as an emulsion in Cremophor® RH40 as described hereinabove (unless otherwise indicated).

### EXAMPLE 30

### Method of Preparation of the Inventive Compounds for In Vitro Studies

To perform the in vitro experiments described by the following examples, the inventive compounds were prepared in the form of an emulsion as described below.

An emulsion comprising an inventive compound and phosphatidylcholine (PC) was prepared as described by Spooner et al. (Spooner, Clark et al. 1988). The inventive compound was mixed with PC in a 4:1 (m/m) ratio in chloroform, the mixture was dried under nitrogen, then vacuum evaporated overnight; the resulting powder was taken up in 0.16 M potassium chloride containing 0.01 M EDTA and the lipid particles were then dispersed by ultrasound for 30 minutes at 37° C. The liposomes so formed were then separated by ultracentrifugation (XL 80 ultracentrifuge, Beckman Coulter, Villepinte, France) at 25,000 rpm for 45 minutes to recover liposomes having a size greater than 100 nm and close to that of chylomicrons. Liposomes composed only of PC were prepared concurrently to use as negative control.

The composition of the liposomes in the inventive compound was estimated by using the enzyme colorimetric triglyceride assay kit. The assay was carried out against a standard curve, prepared with the lipid calibrator CFAS, Ref. 759350 (Boehringer Mannheim GmbH, Germany). The standard curve covered concentrations ranging from 16 to 500 μg/ml. 100 μl of each sample dilution or calibration standard were deposited per well on a titration plate (96 wells). 200 μl of triglyceride reagents (ref. 701912, Boehringer Mannheim GmbH, Germany) were then added to each well, and the entire plate was incubated at 37° C. for 30 minutes. Optical densities (OD) were read on a spectrophotometer at 492 nm. Triglyceride concentrations in each sample were calculated from the standard curve plotted as a linear function y=ax+b, where y represents OD and x represents triglyceride concentrations.

Liposomes containing the inventive compounds, prepared in this manner, were used for in vitro experiments described by the following examples.

### Example 31

### Evaluation of PPAR Activation In Vitro by the Inventive Compounds

Nuclear receptors of the PPAR subfamily which are activated by two major pharmaceutical classes—fibrates and glitazones, widely used in the clinic for the treatment of dyslipidemias and diabetes—play an important role in lipid and glucose homeostasis. The following experimental data show that the inventive compounds activate PPARα in vitro.

PPAR activation was tested in vitro in RK13 fibroblast cell lines or in a hepatocyte line HepG2 by measuring the transcriptional activity of chimeras composed of the DNA binding domain of the yeast gal4 transcription factor and the ligand binding domain of the different PPARs. The example below is given for HepG2 cells.

A—Culture Protocols:

HepG2 cells were from ECACC (Porton Down, UK) and were grown in DMEM medium supplemented with 10% (VN) fetal calf serum, 100 U/ml penicillin (Gibco, Paisley, UK) and 2 mM L-glutamine (Gibco, Paisley, UK). The culture medium was changed every two days. Cells were kept at 37° C. in a humidified 95% air/5% CO₂ atmosphere.

B—Description of Plasmids Used for Transfection:

The plasmids pG5TkpGL3, PRL-CMV, pGal4-hPPARα, pGal4-hPPARγ and pGal4-f have been described by Raspe et al. (Raspe, Madsen et al. 1999). The pGal4-mPPARα and pGal4-hPPARβ constructs were obtained by cloning PCR-amplified DNA fragments corresponding to the DEF domains of the mouse PPARα and human PPARα nuclear receptors, respectively, into the pGal4-f vector.

C—Transfection:

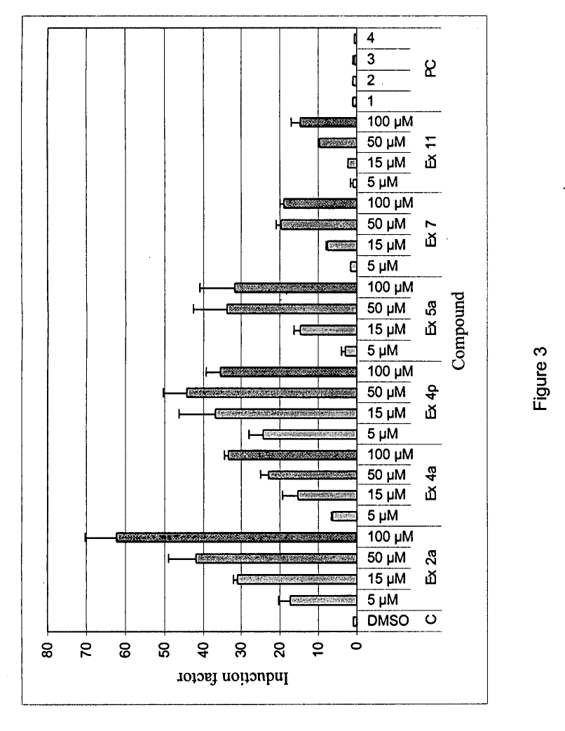

HepG2 cells were seeded in 24-well culture dishes at 5×10⁴ cells/well and transfected for 2 hours with the reporter plasmid pG5TkpGL3 (50 ng/well), the expression vectors pGal4-f, pGal4-mPPARα, pGal4-hPPARα, pGal4-hPPARγ, or pGal4-hPPARβ (100 ng/well) and the transfection efficiency control vector pRL-CMV (1 ng/well) according to the previously described protocol (Raspe, Madsen et al. 1999), then incubated for 36 hours with the test compounds. At the end of the experiment, the cells were lysed (Gibco, Paisley, UK) and luciferase activity was determined with a Dual-Luciferase™ Reporter Assay System kit (Promega, Madison, Wis., USA) according to the supplier's instructions. The protein content of the cell extracts was then measured with the Bio-Rad Protein Assay kit (Bio-Rad, Munich, Germany) as directed by the supplier. The inventors demonstrate an increase in luciferase activity in cells treated with the inventive compounds and transfected with the pGal4-hPPARα plasmid. Said induction of luciferase activity indicates that the inventive compounds are activators of PPARα. FIG. 3 gives an example of the results obtained with the inventive compounds.

FIG. 3: HepG2 cells transfected with Gal4/PPARα plasmids were incubated with different concentrations (5, 15, 50 and 100 μM) of the inventive comopunds (Ex 2a, Ex 4a, Ex 4p, Ex 5a, Ex 7 and Ex 11) for 24 h and with different concentrations of the vehicle (PC) noted 1, 2, 3, 4 as controls for the 5, 15, 50 and 100 μM concentrations of the inventive compounds (according to the 4:1 (m/m) ratio described in example 2 (Method of preparation of the inventive compounds for in vitro studies)). The results are expressed as the induction factor (luminescent signal of treated cells divided by luminescent signal of untreated cells) after the different treatments. The higher the induction factor the more potent the PPARα agonist activity. The results show that inventive compound Ex 2a produced a maximum 62-fold induction of the luminescent signal at 100 μM, 41 at 50 μM, 31 at 15 μM and 17 at 5 μM. Inventive compound Ex 4a also showed a dose-dependent increase in the induction factor of 33 at 100 μM, 23 at 50 μM, 15 at 15 μM and 6 at 5 μM. Inventive compound Ex 4p also induced an increase in the luminescent signal, revealing an activity on the PPARα nuclear receptor. The induction factors for inventive compound Ex 4p were 35 at 100 μM, 44 at 50 μM, 36 at 15 μM and 24 at 5 μM. The induction factors for compound Ex 5a were 32 at 100 μM, 34 at 50 μM, 15 at 15 μM and 4 at 5 μM. Finally, inventive compound Ex 7 induced a 19-fold induction at 100 μM, 19 at 50 μM, 7 at 15 μM and 1.5 at 5 μM. In contrast, when the cells were incubated with the vehicle (PC liposome), no significant induction was observed. These results demonstrate that the inventive compounds tested exhibit significant PPARα ligand activity and therefore enable the transcriptional activation thereof.

### Example 32

### Evaluation of the Anti-Inflammatory Properties of the Inventive Compounds

An inflammatory response is observed in many neurological disorders, such as cerebral ischemias. Inflammation is also an important factor in neurodegeneration. In stroke, one of the first reactions of glial cells is to release cytokines and free radicals. This release of cytokines and free radicals results in an inflammatory response in the brain which can lead to neuron death (Rothwell 1997).

Cell lines and primary cells were cultured as described hereinabove.

Lipopolysaccharide (LPS) bacterial endotoxin (Escherichia coli 0111:B4) (Sigma, France) was reconstituted in distilled water and stored at 4° C. Cells were treated with LPS 1 μg/ml for 24 hours. To avoid interference from other factors, the culture medium was completely changed.

TNF-α is an important factor in the inflammatory response to stress (oxidative stress for example). To evaluate TNF-α secretion in response to stimulation by increasing doses of LPS, the culture medium of stimulated cells was removed and TNF-α was assayed with an ELISA-TNF-α kit (Immunotech, France). Samples were diluted 50-fold so as to be in the range of the standard curve (Chang, Hudson et al. 2000).

The anti-inflammatory property of the compounds was characterized as follows: the cell culture medium was completely changed and the cells were incubated with the test compounds for 2 hours, after which LPS was added to the culture medium at 1 μg/ml final concentration. After a 24-hour incubation, the cell supernatant was recovered and stored at −80° C. when not treated directly. Cells were lysed and protein was quantified with the Bio-Rad Protein Assay kit (Bio-Rad, Munich, Germany) according to the supplier's instructions.

The measurement of the decrease in TNF-α secretion induced by treatment with the test compounds is expressed as pg/ml/μg protein and as the percentage relative to the control. These results show that the inventive compounds have anti-inflammatory properties.

### Example 33

### Evaluation of the Antioxidant Properties of the Inventive Compounds

A—Protection Against LDL Oxidation Induced by Copper:

Oxidation of LDL is an important modification which plays a major role in the onset and development of atherosclerosis (Jurgens, Hoff et al. 1987). The following protocol allows demonstration of the antioxidant properties of compounds. Unless otherwise indicated, all reagents were from Sigma (St Quentin, France).

LDL were prepared as described by Lebeau et al. (Lebeau, Furman et al. 2000). The solutions of the test compounds were prepared at 10⁻² M in ethanol and diluted in PBS so that the final concentration ranged from 0.1 to 100 μM with a total ethanol concentration of 1% (VN).

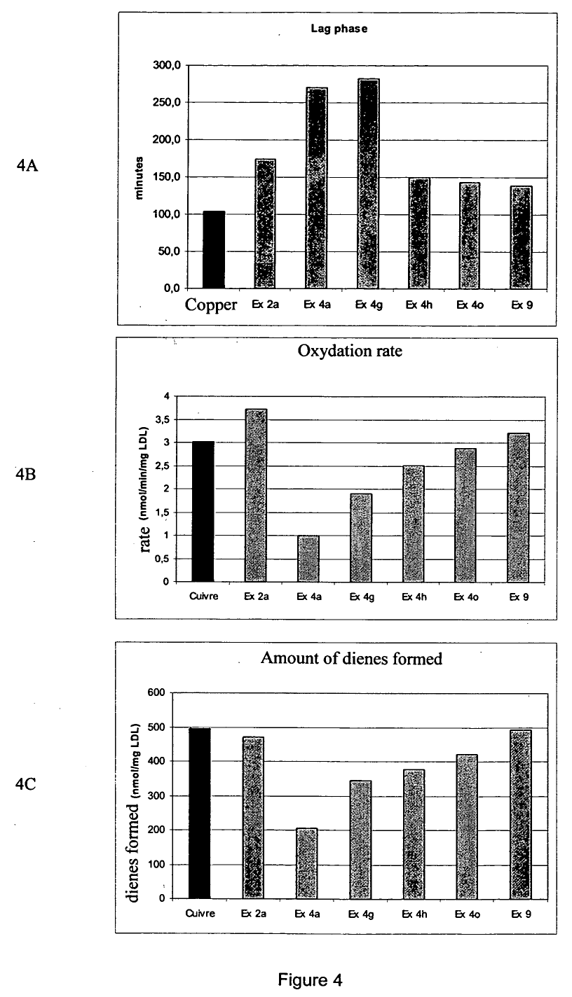

Before oxidation, EDTA was removed from the LDL preparation by dialysis. The oxidation reaction was then carried out at 30° C. by adding 100 μl of 16.6 μM CuSO₄ to 800 μl of LDL (125 μg protein/ml) and 100 μl of a test compound solution. The formation of dienes, the species to be followed, was measured by the optical density at 234 nm in the samples treated with the compounds in the presence or absence of copper. Optical density at 234 nm was measured every 10 minutes for 8 hours on a thermostated spectrophotometer (Kontron Uvikon 930). The analyses were carried out in triplicate. A compound was considered to have antioxidant activity when it shifted the lag phase latency relative to the control sample. The inventors demonstrate that the inventive compounds delayed LDL oxidation (induced by copper), indicating that the inventive compounds possess intrinsic antioxidant activity. FIG. 4 presents an example of the results obtained with the inventive compounds.

FIG. 4a shows that incubation of LDL with the inventive compounds delayed conjugated diene formation. The lag phase was 104 minutes for copper alone as compared with a lag phase for conjugated diene formation that reached 282 minutes when LDL were incubated with inventive compound Ex 4g (inventive compound described in example 4g hereinabove) at 10⁻⁴ M. Inventive compound Ex 4a also increased the lag phase to 270 minutes. Said two compounds induced an increase in the lag phase of 170 and 160%, respectively. Compounds Ex 4h, 4o, 2a and 9 induced a 43, 37, 67 and 33% increase in the lag phase, respectively. This lag in the formation of conjugated dienes is characteristic of antioxidants.

FIG. 4B shows that incubation of the inventive compounds with LDL in the presence of copper slowed the rate of conjugated diene formation. This rate was 3 nmol/min/mg of LDL with copper alone, and decreased to 1 nmol/min/mg of LDL with compound Ex 4a at 10⁻⁴ M, which corresponds to a 66% decrease in the oxidation rate. Inventive compounds Ex 4g and Ex 4h also slowed the LDL oxidation rate which in this case was 1.8 and 2.5 nmol/min/mg of LDL, respectively. Incubation of LDL with inventive compounds Ex 4o, 2a and 9 did not significantly alter the LDL oxidation rate.

FIG. 4C shows that incubation of LDL with copper led to the formation of 496 nmol of conjugated dienes per mg of LDL. Incubation with compound Ex 4a (10⁻⁴ M) led to a 60% decrease in the maximum amount of conjugated dienes formed. Compounds Ex 4g and 4h (10⁻⁴M) also inhibited conjugated diene formation. Incubation of LDL with said compounds led to a respective 31 and 24% decrease in the maximum amount of conjugated dienes formed.

B—Evaluation of the Protection Conferred by the Inventive Compounds Against Lipid Peroxidation:

The inventive compounds which were tested are the compounds whose preparation is described in the hereinabove examples.

LDL oxidation was measured by the TBARS method (Thiobarbituric Acid Reactive Substances).

According to the same principle as that described hereinabove, LDL were oxidized in the presence of CuSO₄ and lipid peroxidation was evaluated as follows:

TBARS were measured by a spectrophotometric method, lipid hydroperoxidation was measured by using lipid peroxide-dependent oxidation of iodide to iodine. The results are expressed as nmol of malondialdehyde (MDA) or as nmol hydroperoxide/mg protein.

The results obtained hereinabove by measuring the inhibition of conjugated diene formation, were confirmed by the experiments measuring LDL lipid peroxidation. The inventive compounds also afforded efficient protection of LDL against lipid peroxidation induced by copper (an oxidizing agent).

### Example 34

### Measurement of the Antioxidant Properties of the Inventive Compounds on Cell Cultures

A—Culture Protocol:

Neuronal, neuroblastoma (human) and PC12 cells (rat) were the cell lines used for this type of study. PC12 cells were prepared from a rat pheochromocytoma and have been characterized by Greene and Tischler (Greene and Tischler, 1976). These cells are commonly used in studies of neuron differentiation, signal transduction and neuron death. PC12 cells were grown as previously described (Farinelli, Park et al. 1996) in complete RPMI medium (Invitrogen) supplemented with 10 % horse serum and 5 % fetal calf serum.

Primary cultures of endothelial and smooth muscle cells were also used. Cells were obtained from Promocell (Promocell GmBH, Heidelberg, Germany) and cultured according to the supplier's instructions.

The cells were treated with different doses of the compounds ranging from 5 to 100 μM for 24 hours. The cells were then recovered and the increase in expression of the target genes was evaluated by semi-quantitative PCR.

B—mRNA Measurement:

mRNA was extracted from the cultured cells treated or not with the inventive compounds. Extraction was carried out with the reagents of the Absolutely RNA RT-PCR miniprep kit (Stratagene, France) as directed by the supplier. mRNA was then assayed by spectrometry and quantified by semi-quantitative RT-PCR on a GeneAmp® PCR System 9700 (Applied Biosystems, USA). Primer pairs specific for the genes encoding the antioxidant enzymes superoxide dismutase (SOD), catalase and glutathione peroxidase (GPx) were used as probes. Primer pairs specific for the β-actin and cyclophilin genes were used as control probes (see Table 1).

An increase in mRNA expression of the antioxidant enzyme genes, measured by semi-quantitative RT-PCR, was demonstrated in the different cell types used, when the cells were treated with the inventive compounds.

TABLE I

| | | Semi-quantitative PCR | | |
|---|---|---|---|---|
| Name | Sequence | Tm | No. cycles | Gene |

| | | | | |
|---|---|---|---|---|
| beta-actin_h_1_s 189 | TTCAACTCCATCA | 55° C. | 25 | β actin |
| | TGAAGTGTGAC | | | |
| beta-actin_h_1_as 188 | TCGTCATACTCCT | | | |
| | TGCTTGCTGATC | | | |
| | C | | | |
| cyclophilin A_h_1_s 513 | GGTGACTTCACA | 50° C. | 20 to 25 | Cyclophilin |
| | CGCCATAATG | | | |
| cyclophilin A_h_1_as | TGTGTTGGGTCC | | | |
| 512 | AGCATTTG | | | |
| SOD1_h_s 558 | CCTCTATCCAGA | 55° C. | 30 | SOD 1 |
| | AAACACGG | | | |
| SOD1_h_as 557 | GCCTCAGACTAC | | | |
| | ATCCAAGG | | | |
| CAT_h_s 1219 | TTGCCTATCCTGA | 55° C. | 25 to 30 | Catalase |
| | CACTCACCG | | | |
| CAT_h_as 1220 | GAATCTCCGCAC | | | |
| | TTCTCCAG | | | |
| GPX_h_s 555 | GAAGTGCGAGGT | | | |
| | GAACGGTG | | | |
| GPX_h_as 554 | TGTCAATGGTCT | 55° C. | 30 | GPx |
| | GGAAGCGG | | | |

C—Control of Oxidative Stress:

Measurement of Oxidizing Species in the Cultured Cells:

The antioxidant properties of the compounds were also evaluated by means of a fluorescent tag the oxidation of which was followed by appearance of a fluorescence signal. The reduction in the intensity of the emitted fluorescence signal was determined in cells treated with the compounds in the following manner: PC12 cells cultured as described earlier (black 96-well plates, transparent bottom, Falcon) were incubated with increasing doses of H₂O₂ (0.25 mM - 1 mM) in serum-free medium for 2 and 24 hours. After incubation, the medium was removed and the cells were incubated with 10 μM dichlorodihydrofluorescein diacetate solution (DCFDA, Molecular Probes, Eugene, USA) in PBS for 30 min at 37° C. in a 5 % CO₂ atmosphere. The cells were then rinsed with PBS. The fluorescence emitted by the oxidation tag was measured on a fluorimeter (Tecan Ultra 384) at an excitation wavelength of 495 nm and an emission wavelength of 535 nm. The results are expressed as the percentage of protection relative to the oxidized control.

Fluorescence intensity was lower in the cells incubated with the inventive compounds than in untreated cells. These findings indicate that the inventive compounds promote inhibition of the production of oxidative species in cells subjected to oxidative stress. The previously described antioxidant properties are also effective at inducing antiradical protection in cultured cells.

D—Measurement of Lipid Peroxidation:

The different cell lines (cell models noted hereinabove) and the primary cell cultures were treated as described earlier. The cell supernatant was recovered after treatment and the cells were lysed and recovered for determination of protein concentration. Lipid peroxidation was detected as follows: lipid peroxidation was measured by using thiobarbituric acid (TBA) which reacts with lipid peroxidation of aldehydes such as malondialdehyde (MDA). After treatment, the cell supernatant was collected (900 μl) and 90 μl of butylated hydroxytoluene were added (Morliere, Moysan et al. 1991). One milliliter of 0.375% TBA solution in 0.25 M hydrochloric acid containing 15% trichloroacetic acid was also added to the reaction medium. The mixture was heated at 80° C. for 15 min, cooled on ice and the organic phase was extracted with butanol. The organic phase was analyzed by spectrofluorimetry (λexc=515 nm and λem=550 nm) on a Shimazu 1501 spectrofluorimeter (Shimadzu Corporation, Kyoto, Japan). TBARS are expressed as MDA equivalents using tetra-ethoxypropane as standard. The results were normalized for protein concentration. The decrease in lipid peroxidation observed in the cells treated with the inventive compounds confirms the previous results.

The inventive compounds advantageously exhibit intrinsic antioxidant properties allowing to slow and/or inhibit the effects of an oxidative stress. The inventors also show that the inventive compounds are capable of inducing the expression of genes encoding antioxidant enzymes. These particular features of the inventive compounds allow cells to more effectively fight against oxidative stress and therefore be protected against free radical-induced damage.

### Example 35

### Evaluation of the Effects of the Inventive Compounds in a Rat Model of Parkinson's Disease

A—Treatment of Animals

1—Animals and Administration of the Compounds

Adult male Wistar rats (280-300 g) were maintained on a 12-hour light/dark cycle at a constant temperature of 20±3° C. Animals had access to food and water ad libitum and weight gain was recorded.

Animals were fed a normal diet or a diet supplemented with the inventive compounds (300 mg/kg per day) for 7 days before induction of the dopaminergic lesion and for 15 days after induction of same.

2—Animal Model of Parkinson's Disease by Selective Damage to Dopaminergic Neurons

6-hydroxydopamine (6-OHDA) is a neurotoxin taken up by dopaminergic neurons via a dopamine transporter. Injection of said compound into striatonigral projections induces selective destruction of dopaminergic neurons, and has allowed the development of many animal models of Parkinson's disease (Bordet et al., 2000).

Seven days after commencement of treatment with the inventive compounds, the rats were stereotactically injected with 6-OHDA (4 μg for 8 min) or buffer (sham rats) in the left part of the median tract of the telencephalon to induce striatonigral denervation.

B—Evaluation of the Effect of the Inventive Compounds on the Dopaminergic Lesion

1—Behavioral Sensitization to Apomorphine

1-1 Sensitization Test

Apomorphine is a dopaminergic agonist which stimulates D1 and D2 receptors. The intensity of rotational behavior is an index allowing to measure the severity of the striatonigral lesion. At the end of the treatment, rats received an intraperitoneal injection of apomorphine and rotational behavior was evaluated 15 minutes after sensitization and for a 10 minute period.

1-2 Results

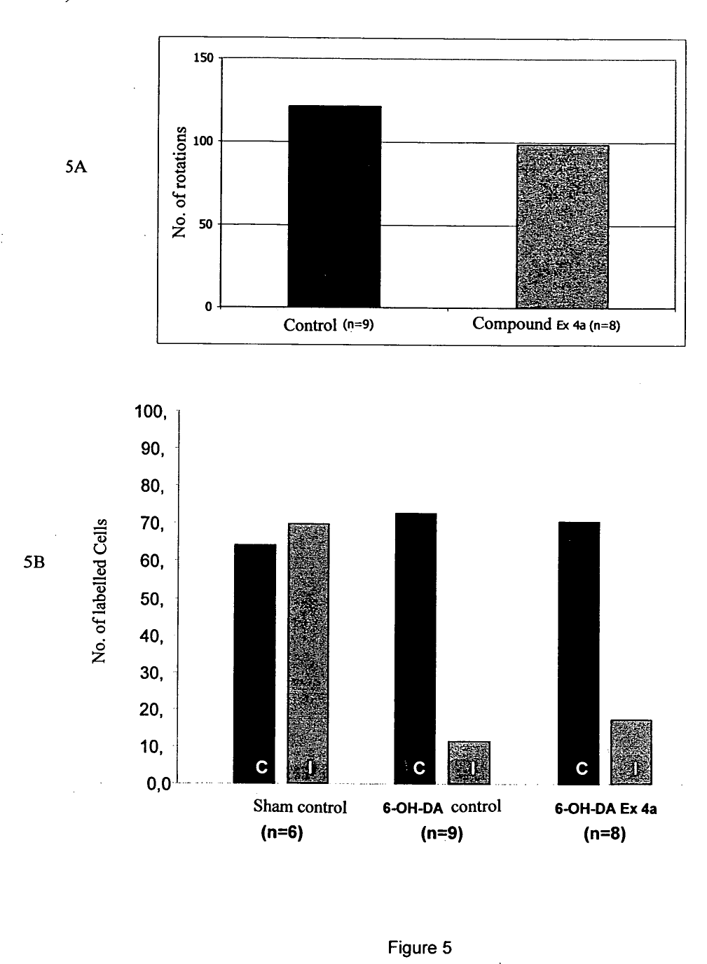

The frequency of rotations increased after the 6-OHDA-induced lesion. The neuroprotective activity of a compound is therefore manifested as a decrease in the number of rotations. The inventive compounds produced a decrease in the number of rotations after apomorphine injection. In fact, the fewer the rotations, the smaller the lesion. These results therefore show that compound Ex 4a has prophylactic and curative properties in a Parkinson's disease model (FIG. 5A).

2- Immunohistochemistry Using an Anti-TH Antibody

Tyrosine hydroxylase (TH) is an enzyme which catalyzes the transformation of tyrosine to dopamine. It is used to label dopaminergic neurons. After the apomorphine sensitization test, the animals were sacrificed and the brains were removed. Brain slices were incubated with anti-TH antibody (SCBT, Santa Cruz, Calif.) and then with a second biotinylated antibody. Visualization was with the ABC staining system kit (Tebu) according to the supplier's instructions.

Viable cells labelled with anti-TH antibody (TH+ cells) were counted. Injection of 6-OHDA induced a selective loss of neurons in the ventral tegmental area (VTA) and substantia nigra (compare the number of TH+ neurons between the ipsilateral and controlateral zone in rats injected with 6-OHDA and between ipsilateral zones in sham rats and rats treated with 6-OHDA (FIG. 5B)).

Rats treated with inventive compound Ex 4a had a greater number of neurons than rats fed a normal diet. The efficacy of the inventive compounds on survival of dopaminergic neurons is demonstrated.

### BIBLIOGRAPHIC REFERENCES

Adams, E. P., F. P. Doyle, et al. (1960). “Antituberculous sulphur compounds. Part IV. Some dimercaptopropyl esters and related dithiouronium bromides.” J Chem Soc: 2674-80.
Adams, H. P., Jr. (2002). “Emergent use of anticoagulation for treatment of patients with ischemic stroke.” Stroke 33(3): 856-61.
Antoniadou-Vyzas, A., G. B. Foscolos, et al. (1986). “Di-adamantane derivatives of a,o-polymethylenediamines with antimicrobial activity.” Eur J Med Chem Chim Ther 21(1): 73-74.
Aveta, R., A. Brandt, et al. (1986). “Syntheses of 2-mercapto- and 2-alkylthio-1,3-propanediol derivatives.” Gazz Chim Ital 116(11): 649-652.
Bhatia, S. K. and J. Hajdu (1987). “Stereospecific synthesis of 2-thiophosphatidylcholines; a new class of biologically active phospholipid analogues.” Tetrahedron Lett 28(33): 3767-3770.
Bisaglia, M., V. Venezia, et al. (2002). “Acetaminophen protects hippocampal neurons and PC12 cultures from amyloid beta-peptides induced oxidative stress and reduces NF-kappaB activation.” Neurochem Int 41(1): 43-54.
Bodai, Novak, et al. (1999). “Synthesis and lipase-catalyzed enantiope selective acetylation of 2-benzoyloxy-1,3-propanediol.” Synlett 6: 759-761.
Boka, G., P. Anglade, et al. (1994). “Immunocytochemical analysis of tumor necrosis factor and its receptors in Parkinson's disease.” Neurosci Lett 172(1-2): 151-4.
Bordet, R., S. Ridray, et al. (2000). “Involvement of the direct striatonigral pathway in levodopa-induced sensitization in 6-hydroxydopamine-lesioned rats.” Eur J Neurosci 12(6): 2117-23.
Breidert, T., J. Callebert, et al. (2002). “Protective action of the peroxisome proliferator-activated receptor-{gamma} agonist pioglitazone in a mouse model of Parkinson's disease.” J. Neurochem 82(3): 615-624.
Casper, D., U. Yaparpalvi, et al. (2000). “Ibuprofen protects dopaminergic neurons against glutamate toxicity in vitro.” Neurosci Lett 289(3): 201-4.
Chang, R. C., P. Hudson, et al. (2000). “Influence of neurons on lipopolysaccharide-stimulated production of nitric oxide and tumor necrosis factor-alpha by cultured glia.” Brain Res 853(2): 236-44.
Combs, C. K., P. Bates, et al. (2001). “Regulation of beta-amyloid stimulated proinflammatory responses by peroxisome proliferator-activated receptor alpha.” Neurochem Int 39(5-6): 449-57.
Combs, C. K., D. E. Johnson, et al. (2000). “Inflammatory mechanisms in Alzheimer's disease: inhibition of beta- amyloid-stimulated proinflammatory responses and neurotoxicity by PPARgamma agonists.” J Neurosci 20(2): 558-67.
Cullingford, T., K. Bhakoo, et al. (1998). “Distribution of mRNAs encoding the peroxisome proliferator-activated receptor alpha, beta, and gamma and the retinoid X receptor alpha, beta, and gamma in rat central nervous system.” J. Neurochem 70(4): 1366-1375.
Daniher, A. and J. Bashkin (1998). “Precise control of RNA cleavage by ribozyme mimics.” Chem Commun 10: 1077-1078.
Daubert, Spiegl, et al. (1943). “Unsaturated synthetic glycerides. II. Unsymmetrical dioleo-monosaturated triglycerides.” J Am Chem Soc 65: 2145.
Delerive, P., K. De Bosscher, et al. (1999). “Peroxisome proliferator-activated receptor alpha negatively regulates the vascular inflammatory gene response by negative cross-talk with transcription factors NF-kappaB and AP-1.” J Biol Chem 274(45): 32048-54.
Delerive, P., J. C. Fruchart, et al. (2001). “Peroxisome proliferator-activated receptors in inflammation control.” J Endocrinol 169(3): 453-9.
Devchand, P. R., H. Keller, et al. (1996). “The PPARalpha-leukotriene B4 pathway to inflammation control.” Nature 384(6604): 39-43.
Dexter, D., C. Carter, et al. (1989). “Basal lipid peroxidation in substantia nigra is increased in Parkinson's disease.” J. Neurochem 52(2): 381-389.
Diab, A., C. Deng, et al. (2002). “Peroxisome Proliferator-Activated Receptor-{gamma} Agonist 15-Deoxy-(Delta}1 2,1412,14-Prostaglandin J2 Ameliorates Experimental Autoimmune Encephalomyelitis.” J Immunol. 168(5): 2508-2515.
Farinelli, S. E., D. S. Park, et al. (1996). “Nitric oxide delays the death of trophic factor-deprived PC12 cells and sympathetic neurons by a cGMP-mediated mechanism.” J Neurosci 16(7): 2325-34.
Feuge, R. O., A. T. Gros, et al. (1953). “Modification of vegetable oils. XIV. Properties of aceto-oleins.” J Am Oil Chem Soc 30: 320-325.
Feuge, R. O. and N. V. Lovegren (1956). “Dilatometric properties of some butyropalmitins, butyrostearins, and acetopalmitins.” J Am Oil Chem Soc 33: 367-372.
Gaffney, P. R. J. and C. B. Reese (1997). “Preparation of 2-O-arachidonoyl-1-O-stearoyl-sn-glycerol and other di-O-acyl glycerol derivatives.” Tetrahedron Lett 38(14): 2539-2542.
Gangadhar, A., R. Subbarao, et al. (1989). “A facile synthesis of 1(3)-acylglycerols.” Synth Commun 19: 2505-2514.
Greene, L. A. and A. S. Tischler (1976). “Establishment of a noradrenergic clonal line of rat adrenal pheochromocytoma cells which respond to nerve growth factor.” Proc Natl Acad Sci USA 73(7): 2424-8.
Griffin, W., J. Sheng, et al. (1998). “Glial-neuronal interactions in Alzheimer's disease: the potential role of a ‘cytokine cycle’ in disease progression.” Brain Pathol 8(1): 65-72.
Gronowitz, S., B. Herslöf, et al. (1978). “Syntheses and chroptical properties of some derivatives of 1-thioglycerol.” Chem Phys Lipids 22: 307-320.
Han, S. Y., S. H. Cho, et al. (1999). “Monoacetyldiglycerides as new Ca2+ mobilizing agents in rat pancreatic acinar cells.” Bioorg Med Chem Lett 9(1): 59-64.
Harada, H., T. Morie, et al. (1996). “An efficient synthesis of 6-substituted aminohexahydro-1H-1,4-diazepines from 2-substituted aminopropenals.” Chem Pharm Bull 44(12): 2205-2212.
Heneka, M. T., T. Klockgether, et al. (2000). “Peroxisome proliferator-activated receptor-gamma ligands reduce neuronal inducible nitric oxide synthase expression and cell death in vivo.” J Neurosci 20(18): 6862-7.
Ho, L., C. Pieroni, et al. (1999). “Regional distribution of cyclooxygenase-2 in the hippocampal formation in Alzheimer's disease.” J Neurosci Res 57(3): 295-303.
Hohlfeld, R. (1997). “Biotechnological agents for the immunotherapy of multiple sclerosis. Principles, problems and perspectives.” Brain 120(Pt 5): 865-916.
Huell, M., S. Strauss, et al. (1995). “Interleukin-6 is present in early stages of plaque formation and is restricted to the brains of Alzheimer's disease patients.” Acta Neuropathol (Berl) 89(6): 544-51.
Hunot, S., B. Brugg, et al. (1997). “Nuclear translocation of NF-kappaB is increased in dopaminergic neurons of patients with parkinson disease.” Proc Natl Acad Sci USA 94(14): 7531-6.
Jurgens, G., H. F. Hoff, et al. (1987). “Modification of human serum low density lipoprotein by oxidation—characterization and pathophysiological implications.” Chem Phys Lipids 45(2-4): 315-36.
Kainu, T., A. C. Wikstrom, et al. (1994). “Localization of the peroxisome proliferator-activated receptor in the brain.” Neuroreport 5(18): 2481-5.
Katoch, R., G. K. Trivedi, et al. (1999). “1-[2-Hydroxy-3-octadecan-1′-oate]propyl-2″,2″,5″,5″-tetramethyl pyrolidine-N-oxyl-3″-carboxylate as a potential spin probe for membrane structure studies.” Bioorg Med Chem 7(12): 2753-8.
Khanolkar, A. D., V. Abadji, et al. (1996). “Head group analogs of arachidonylethanolamide, the endogenous cannabinoid ligand.” J Med Chem 39(22): 4515-9.
Kitamura, Y., S. Shimohama, et al. (1999). “Increased expression of cyclooxygenases and peroxisome proliferator-activated receptor-gamma in Alzheimer's disease brains.” Biochem Biophvs Res Commun 254(3): 582-6.
Kitchin, J., R. C. Bethell, et al. (1994). “Synthesis and structure-activity relationships of a series of penicillin-derived HIV proteinase inhibitors: heterocyclic ring systems containing P1′ and P2′ substituents.” J Med Chem 37(22): 3707-16.
Kotsovolou, S., A. Chiou, et al. (2001). “Bis-2-oxo amide triacylglycerol analogues: a novel class of potent human gastric lipase inhibitors.” J Org Chem 66(3): 962-7.
Kürfurst, R., V. Roig, et al. (1993). “Oligo-a-deoxyribonucleotides with a modified nucleic base and covalently linked to reactive agents.” Tetrahedron 49(32): 6975-6990.
Landolfi, C., L. Soldo, et al. (1998). “Inflammatory molecule release by beta-amyloid-treated T98G astrocytoma cells: role of prostaglandins and modulation by paracetamol.” Eur J Pharmacol 360(1): 55-64.
Lebeau, J., C. Furman, et al. (2000). “Antioxidant properties of di-tert-butylhydroxylated flavonoids.” Free Radic Biol Med 29(9): 900-12.
Lehmann, J. M., J. M. Lenhard, et al. (1997). “Peroxisome proliferator-activated receptors alpha and gamma are activated by indomethacin and other non-steroidal anti-inflammatory drugs.” J Biol Chem 272(6): 3406-10.
Lim, G. P., F. Yang, et al. (2000). “Ibuprofen Suppresses Plaque Pathology and Inflammation in a Mouse Model for Alzheimer's Disease.” J. Neurosci 20(15): 5709-5714.
Mackenzie, I. and D. Munoz (1998). “Nonsteroidal anti-inflammatory drug use and Alzheimer-type pathology in aging.” Neurology 50(4): 986-990.
Maeshiba, Y., Y. Kiyota, et al. (1997). “Disposition of the new antidiabetic agent pioglitazone in rats, dogs, and monkeys.” Arzneimittelforschung 47(1): 29-35.
Marx, M. H., C. Piantadosi, et al. (1988). “Synthesis and evaluation of neoplastic cell growth inhibition of 1-N-alkylamide analogues of glycero-3-phosphocholine.” J Med Chem 31(4): 858-63.
McGeer, P., M. Schulzer, et al. (1996). “Arthritis and anti-inflammatory agents as possible protective factors for Alzheimer's disease: a review of 17 epidemiologic studies.” Neurology 47(2): 425-432.
Mogi, M., M. Harada, et al. (1994). “Interleukin-1 beta, interleukin-6, epidermal growth factor and transforming growth factor-alpha are elevated in the brain from parkinsonian patients.” Neurosci Lett 180(2): 147-50.
Morliere, P., A. Moysan, et al. (1991). “UVA-induced lipid peroxidation in cultured human fibroblasts.” Biochim Biophvs Acta 1084(3): 261-8.
Morris, A. D., G. Atassi, et al. (1997). “The synthesis of novel melphalan derivatives as potential antineoplastic agents.” Eur J Med Chem 32(4): 343-50.
Murata, M., S. Ikoma, et al. (1991). “New synthesis of 2-thio-PAF and related compounds as substrates of PAF acetylhydrolase and phospholipase A2.” Chem Pharm Bull 39(5): 1335-1336.
Natarajan, C. and J. J. Bright (2002). “Peroxisome proliferator-activated receptor-gamma agonists inhibit experimental allergic encephalomyelitis by blocking IL-12 production, IL-12 signaling and Th1 differentiation.” Genes Immun 3(2): 59-70.
Nazih, A., Y. Cordier, et al. (1999). “Synthesis and stability study of the new pentaammonio lipidpcTG90, a gene transfer agent.” Tetrahedron Lett 40(46): 8089-92.
Nazih, A., Y. Cordier, et al. (2000). “One-pot transformation of a t-butyl carbamate to a bromoacetamide in the synthesis of the gene transfer agent pcTG201.” Synlett 5: 635-6.
Paris, G. Y., D. L. Garmaise, et al. (1980). “Glycerides as prodrugs. 3. Synthesis and antiinflammatory activity of [1-(p-chlorobenzoyl)-5-methoxy-2-methylindole-3-acetyl]glycerides (indomethacin glycerides).” J Med Chem 23(1): 9-13.
Poynter, M. E. and R. A. Daynes (1998). “Peroxisome proliferator-activated receptor alpha activation modulates cellular redox status, represses nuclear factor-kappaB signaling, and reduces inflammatory cytokine production in aging.” J Biol Chem 273(49): 32833-41.
Putnam, W. and J. Bashkin (2000). “De novo synthesis of artificial ribonucleases with benign metal catalysts.” Chem Commun 9: 767-768.
Rahman, M. D., D. L. Ziering, et al. (1988). “Effects of sulfur-containing analogues of stearic acid on growth and fatty acid biosynthesis in the protozoan Crithidia fasciculata.” J Med Chem 31(8): 1656-9.
Ramalingan, K., N. Raju, et al. (1995). “Synthesis of nitroimidazole substituted 3,3,9,9-tetramethyl-4,8-diaza-undecane-2,10-dione dioximes (propylene amine oximes, PnAOs); ligands for technetium-99m complexes with potential for imaging hypoxic tissue.” Tetrahedron 51(10): 2875-94.
Raspe, E., L. Madsen, et al. (1999). “Modulation of rat liver apolipoprotein gene expression and serum lipid levels by tetradecylthioacetic acid (TTA) via PPARalpha activation.” J Lipid Res 40(11): 2099-110.
Rich, J., D. Rasmusson, et al. (1995). “Nonsteroidal anti-inflammatory drugs in Alzheimer's disease.” Neurology 45(1): 51-55.
Robinson, N. (1960). “Micellar size and surface activity of some C18 a-monoglycerides in benzene.” J Pharm Pharmacol 12: 685-689.
Rogers, J., L. Kirby, et al. (1993). “Clinical trial of indomethacin in Alzheimer's disease.” Neurology 43(8): 1609-1611.
Rothwell, N. J. (1997). “Cytokines and acute neurodegeneration.” Mol Psychiatry 2(2): 120-1.
Saggu, H., J. Cooksey, et al. (1989). “A selective increase in particulate superoxide dismutase activity in parkinsonian substantia nigra.” J. Neurochem 53(3): 692-697.
Scriba, G. K. (1993). “Phenytoin-lipid conjugates as potential prodrugs of phenytoin.” Arch Pharm (Weinheim) 326(8): 477-81.
Seltzman, Fleming, et al. (2000). “Facile synthesis and stabilization of 2-arachidonylglycerol via its 1,3-phenylboronate ester.” Tetrahedron Lett 41(19): 3589-3592.
Shaban, M. (1977). “Determination of the position of linkage of 2-acetamido-2-deoxy-D-galactose and 2-acetamido-2-deoxy-D-glucose residues in oligosaccharides and glycoproteins. Synthesis of 2-acetamido-2-deoxy-D-xylitol and 2-acetamido-2-deoxy-L-threitol.” Carbohydr Res 59: 213-233.
Shealy, Y. F., J. L. Frye, et al. (1984). “Synthesis and properties of some 13-cis- and all-trans-retinamides.” J Pharm Sci 73(6): 745-51.
Spooner, P. J., S. B. Clark, et al. (1988). “The ionization and distribution behavior of oleic acid in chylomicrons and chylomicron-like emulsion particles and the influence of serum albumin.” J Biol Chem 263(3): 1444-53.
Staels, B., W. Koenig, et al. (1998). “Activation of human aortic smooth-muscle cells is inhibited by PPARalpha but not by PPARgamma activators.” Nature 393(6687): 790-3.
Stewart, W., C. Kawas, et al. (1997). “Risk of Alzheimer's disease and duration of NSAID use.” Neurology 48(3): 626-632.
Strawn, L. M., R. E. Martell, et al. (1989). “Synthesis and evaluation of iodinated analogues of diacylglycerols as potential probes for protein kinase C.” J Med Chem 32(3): 643-8.
Terradas (1993). “Marked dependence of enzyme prochiral selectivity on the solvent.” J Amer Chem Soc 115: 390-396.
Urakami, C. and K. Kakeda (1953). “Derivatives of dl-aminopropanediols.” Bull Chem Soc Jpn 26(5): 276-278.

1-24. (canceled)

25. A method for the treatment or prophylaxis of a neurodegenerative pathology, by administering to a subject in need of such treatment or prophylaxis an efficient amount of at least one compound represented by general formula (I) 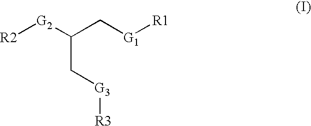
in which:
G1 represents an oxygen atom or a N—R group, G2 and G3 independently represent an oxygen atom, a sulfur atom or a N—R4 group, G1, G2 and G3 not simultaneously representing a N—R or N—R4 group,
R and R4 independently represent a hydrogen atom or a linear or branched alkyl group, saturated or not, optionally substituted, containing from 1 to 5 carbon atoms,
R1, R2 and R3, which are the same or different, represent a hydrogen atom, a CO—R5 group or a group corresponding to the formula CO—(CH₂)₂ₙ₊₁—X—R6, at least one of the groups R1, R2 and R3 being a group corresponding to the formula CO—(CH₂)₂ₙ₊₁—X—R6,
R5 is a linear or branched alkyl group, saturated or not, optionally substituted, possibly comprising a cyclic group, the main chain of which contains from 1 to 25 carbon atoms,
R6 is a linear or branched alkyl group, saturated or not, optionally substituted, possibly comprising a cyclic group, the main chain of which contains from 3 to 23 carbon atoms, preferably 10 to 23 carbon atoms and optionally one or more heterogroups, selected in the group consisting of an oxygen atom, a sulfur atom, a selenium atom, a SO group and a SO₂ group,
X is a sulfur atom, a selenium atom, a SO group or a SO₂ group,
n is a whole number comprised between 0 and 11,
and the optical and geometrical isomers, racemates, salts, hydrates thereof and the mixtures thereof.

26. The method according to claim 25, wherein G1 represents a N—R group.

27. The method according to claim 25, wherein G1 represents a N—R group and G2R2 and G3R3 are not simultaneously hydroxyl groups.

28. The method according to claim 25, wherein G1 and G3 represent oxygen atoms.

29. The method according to claim 25, wherein a single one of the groups R1, R2 or R3 represents a hydrogen atom.

30. The method according to claim 25, wherein two of the groups R1, R2 or R3 represent a hydrogen atom.

31. The method according to claim 25, wherein, in the CO—(CH₂)₂ₙ₊₁—X—R6 group, X represents a sulfur or selenium atom and advantageously a sulfur atom.

32. The method according to claim 25, wherein, in the CO—(CH₂)₂ₙ₊₁—X—R6 group, n is comprised between 0 and 3, more specifically comprised between 0 and 2 and in particular is equal to 0.

33. The method according to claim 25, wherein R6 contains one or more heterogroups, preferably 0, 1 or 2, more preferably 0 or 1, selected in the group consisting of an oxygen atom, a sulfur atom, a selenium atom, a SO group and a SO₂ group.

34. The method according to claim 25, wherein the group CO—(CH₂)₂ₙ₊₁—X—R6 is the CO—CH₂—S—C₁₄H₂₉ group.

35. The method according to claim 25, wherein at least one of the groups R1, R2 and R3 represents a CO—(CH₂)₂ₙ₊₁—X—R6 group in which X represents a sulfur or selenium atom and preferably a sulfur atom and/or R6 is a saturated and linear alkyl group preferably containing from 13 to 20 carbon atoms, preferably 14 to 17, more preferably 14 to 16, and even more preferably 14 carbon atoms.

36. The method according to claim 25, wherein at least two of the groups R1, R2 and R3 are CO—(CH₂)₂ₙ₊₁—X—R6 groups, which are the same or different, in which X represents a sulfur or selenium atom and preferably a sulfur atom.

37. The method according to claim 25, wherein G1 and G3 represent oxygen atoms and G2 represents an oxygen atom or a N—R4 group, preferably an oxygen atom.

38. The method according to claim 25, wherein G1 and G3 represent oxygen atoms, G2 represents an oxygen atom or a N—R4 group, preferably an oxygen atom, and R2 represents a CO—(CH₂)₂ₙ₊₁—X—R6 group.

39. The method according to claim 25, wherein G1 and G3 represent oxygen atoms, G2 is a N—R4 group, and R4 represents a hydrogen atom or a methyl group.

40. The method according to claim 25, wherein G1 represents a N—R group and G2 represents an oxygen or sulfur atom, and preferably an oxygen atom, with R2 advantageously representing a group corresponding to the formula CO—(CH₂)₂ₙ₊₁—X—R6.

41. The method according to claim 25, wherein G1 and G3 represent oxygen atoms and G2 is an oxygen atom and/or R2 represents a CO—(CH₂)₂ₙ₊₁—X—R6 group.

42. The method according to claim 25, wherein G1 represents a N—R group and G3 is a N—R4 group, in which R4 is a hydrogen atom or a methyl group, G2 is an oxygen atom, and/or R2 represents a CO—(CH₂)₂ₙ₊₁—X—R6 group.

43. The method according to claim 25, wherein R1, R2 and R3, which are the same or different, preferably the same, represent (i) a CO—(CH₂)₂ₙ₊₁—X—R6 group, in which X represents a sulfur or selenium atom and preferably a sulfur atom and/or R6 is a saturated and linear alkyl group containing from 13 to 17 carbon atoms, preferably 14 to 16 carbon atoms, even more preferably 14 carbon atoms, in which n is preferably comprised between 0 and 3, and in particular is equal to 0.

44. The method according to claim 25, wherein the compounds represented by formula (I) are selected in the group consisting of:
1-tetradecylthioacetylglycerol;
2-tetradecylthioacetylglycerol;
1,2,3-tritetradecylthioacetylglycerol;
1,2,3-tri-(4-dodecylthio)butanoylglycerol;
1,2,3-tri-(6-decylthio)hexanoylglycerol;
1,2,3-tritetradecylsulfoxyacetylglycerol;
1,2,3-tri-(tetradecylsulfonyl)acetylglycerol;
1,2,3-tri-tetradecylselenoacetylglycerol;
1,3-dipalmitoyl-2-tetradecylthioacetylglycerol;
1,3-dilinoleoyl-2-tetradecylthioacetylglycerol;
1,3-distearyl-2-tetradecylthioacetylglycerol;
1,3-oleoyl-2-tetradecylthioacetylglycerol;
1,3-ditetradecanoyl-2-tetradecylthioacetylglycerol;
1-palmitoyl-2,3-ditetradecylthioacetylglycerol;
1-oleoyl-3-palmitoyl-2-tetradecylthioacetylglycerol;
1,3-dipalmitoyl-2-docosylthioacetylglycerol;
2-tetradecylthioacetamidopropane-1,3-diol;
2-tetradecylthioacetamido-1,3-ditetradecylthioacetyloxypropane;
1,3-ditetradecylthioacetyl-2-palmitoylglycerol;
1,3-diacetyl-2-tetradecylthioacetylglycerol;
1,3-dioctanoyl-2-tetradecylthioacetylglycerol;
1,3-diundecanoyl-2-tetradecylthioacetylglycerol;
1,3-ditetradecylthioacetoxy-2-(tetradecylthiomethyl)carbonylthiopropane;
3-(tetradecylthioacetylamino)propane-1,2-diol;
1-tetradecylthioacetylamino-2,3-(dipalmitoyloxy)propane;
3-tetradecylthioacetylamino-1,2-(ditetradecylthioacetyloxy)propane;
3-palmitoylamino-1,2-(ditetradecylthioacetyloxy)propane;
1,3-di(tetradecylthioacetylamino)propan-2-ol;
1,3-diamino-2-(tetradecylthioacetyloxy) propane;
1,3-ditetradecylthioacetylamino-2-(tetradecylthioacetyloxy)propane;
1,3-dioleoylamino-2-(tetradecylthioacetyloxy)propane;
1,3-ditetradecylthioacetylamino-2-(tetradecylthioacetylthio)propane; and
1-tetradecylthioacetylamino-2,3-di(tetradecylthioacetylthio)propane.

45. The method according to claim 25, wherein the neurodegenerative pathology is Parkinson's disease, Alzheimer's disease or multiple sclerosis.

46. A pharmaceutical composition comprising, in a pharmaceutically acceptable support, at least one compound represented by formula (I) such as defined in claim 25 in association with at least one compound selected in the group consisting of: Castor oil polyoxyl hydrogenated, polyoxyl 35 Castor oil, polyethylene glycol 660 12-hydroxystearate and polysorbate 60.

47. The pharmaceutical composition according to claim 46, for the treatment of a neurodegenerative pathology.

48. The pharmaceutical composition according to claim 46, for the treatment of Parkinson's disease, Alzheimer's disease or multiple sclerosis.
